SEQUENCE LISTING (58 sequences)

SEQ ID NO: 1 - DNA, 2341 bp, synthetic construct
  source 1..2341: mol_type = other DNA
agcgaaagcaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaacctttggccctgtccattttagaaaccaagtcaaaatacgtcgaagagttgacataaatcctggtcatgcagatctcagtgccaaagaggcacaggatgtaatcatggaagttgttttccctaacgaagtgggagccaggatactaacatcggaatcgcaactaacgacaaccaaagagaagaaagaagaactccagggttgcaaaatttctcctctgatggtggcatacatgttggagagagaactggtccgcaaaacgagattcctcccagtggctggtggaacaagcagtgtgtacattgaagtgttgcatttgacccaaggaacatgctgggaacagatgtacactccaggaggggaggcgaggaatgatgatgttgatcaaagcttaattattgctgctagaaacatagtaagaagagccacagtatcagcagatccactagcatctttattggagatgtgccacagcacgcagattggtggaataaggatggtaaacatccttaggcagaacccaacagaagagcaagccgtggatatttgcaaggctgcaatgggactgagaattagctcatccttcagttttggtggattcacatttaagagaacaagcggatcatcagtcaagagagaggaagaggtgcttacgggcaatcttcagacattgaagataagagtgcatgagggatatgaagagttcacaatggttgggagaagagcaacagctatactcagaaaagcaaccaggagattgattcagctgatagtgagtgggagagacgaacagtcgattgccgaagcaataattgtggccatggtattttcacaagaggattgtatgataaaagcagttagaggtgacctgaatttcgtcaatagggcgaatcagcgattgaatcccatgcaccaacttttgagacattttcagaaggatgcaaaggtgctctttcaaaattggggaattgaatccatcgacaatgtgatgggaatgatcgggatattgcccgacatgactccaagcaccgagatgtcaatgagaggagtgagaatcagcaaaatgggggtagatgagtattccagcgcggagaagatagtggtgagcattgaccgttttttgagagttagggaccaacgtgggaatgtactactgtctcccgaggagatcagtgaaacacagggaacagagaaactgacaataacttactcatcgtcaatgatgtgggagattaatggtcctgaatcagtgttggtcaatacctatcagtggatcatcagaaactgggaaactgttaaaattcagtggtcccagaatcctacaatgctgtacaataaaatggaatttgagccatttcagtctttagttccaaaggccgttagaggccaatacagtgggtttgtgagaactctgttccaacaaatgagggatgtgcttgggacatttgataccgctcagataataaaacttcttcccttcgcagccgctccaccaaagcaaagtagaacgcagttctcctcattgactataaatgtgaggggatcaggaatgagaatacttgtaaggggcaattctccagtattcaactacaacaagaccactaaaagactcacagttctcggaaaggatgctggccctttaactgaagacccagatgaaggcacagctggagttgagtccgcagttctgagaggattcctcattctgggcaaagaagacaggagatatggaccagcattaagcataaatgaactgagcaaccttgcgaaaggagagaaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 2 - DNA, 2341 bp, synthetic construct
  source 1..2341: mol_type = other DNA
agcgaaagcaggcaaaccatttgaatggatgtcaatccgactttacttttcttaaaagtgccagcacaaaatgctataagcacaactttcccttatactggagaccctccttacagccatgggacaggaacaggatacaccatggatactgtcaacaggacacatcagtactcagaaaggggaagatggacaacaaacaccgaaactggagcaccgcaactcaacccgattgatgggccactgccagaagacaatgaaccaagtggttatgcccaaacagattgtgtattggaagcaatggccttccttgaggaatcccatcctggtatctttgagacctcgtgtcttgaaacgatggaggttgttcagcaaacacgagtggacaagctgacacaaggccgacagacctatgactggactctaaataggaaccagcctgctgcaacagcattggccaacacaatagaagtgttcagatcaaatggcctcacggccaatgaatctggaaggctcatagacttccttaaggatgtaatggagtcaatgaacaaagaagaaatggagatcacaactcattttcagagaaagagacgagtgagagacaatatgactaagaaaatggtgacacagagaacaataggtaaaaggaagcagagattgaacaaaaggagttatctaattagggcattaaccctgaacacaatgaccaaagatgctgagagagggaagctaaaacggagagcaattgcaaccccagggatgcaaataagggggtttgtatactttgttgagacactagcaaggagtatatgtgagaaacttgaacaatcaggattgccagttggaggcaatgagaagaaagcaaagttggcaaatgttgtaaggaagatgatgaccaattctcaggacactgaaatttctttcaccatcactggagataacaccaaatggaacgaaaatcagaaccctcggatgtttttggccatgatcacatatataaccagaaatcagcccgaatggttcagaaatgttctaagtattgctccaataatgttctcaaacaaaatggcgagactgggaaaggggtacatgtttgagagcaagagtatgaaaattagaactcaaatacctgcagaaatgctagcaagcatcgatttgaaatacttcaatgattcaactagaaagaagattgaaaaaatccggccgctcttaatagatgggactgcatcattgagccctggaatgatgatgggcatgttcaatatgttaagtactgtattaggcgtctccatcctgaatcttggacaaaagagacacaccaagactacttactggtgggatggtcttcaatcttctgatgattttgctctgattgtgaatgcacccaatcatgaagggattcaagccggagtcaacaggttttatcgaacctgtaagctacttggaattaatatgagcaagaaaaagtcttacataaacagaacaggtacatttgaattcacaagttttttctatcgttatgggtttgttgccaatttcagcatggagcttcccagctttggggtgtctgggatcaacgagtctgcggacatgagtattggagttactgtcatcaaaaacaatatgataaacaatgatcttggtccagcaaccgctcaaatggcccttcagctgttcatcaaagattacaggtacacgtaccggtgccatagaggtgacacacaaatacaaacccgaagatcatttgaaataaagaaactgtgggagcaaacccattccaaagctggactgctggtctccgacggaggcccaaatttatacaacattagaaatctccacattcctgaagtctgcttgaaatgggaattaatggatgaggattaccaggggcgtttatgcaacccactgaacccatttgtcaaccataaagacattgaatcagtgaacaatgcagtgataatgccagcacatggtccagccaaaaacatggagtatgatgctgttgcaacaacacactcctggatccccaaaagaaatcgatccatcttgaatacaagccaaagaggaatacttgaagatgaacaaatgtaccaaaagtgctgcaacttatttgaaaaattcttccccagcagttcatacagaagaccagtcgggatatccagtatggtggaggctatggtttccagagcccgaattgatgcacgaattgatttcgaatctggaaggataaagaaagaggagttcactgagatcatgaagatctgttccaccattgaagagctcagacggcaaaaatagtgaatttagcttgtccttcatgaaaaaatgccttgtttctact

SEQ ID NO: 3 - DNA, 2233 bp, synthetic construct
  source 1..2233: mol_type = other DNA
agcgaaagcaggtactgattcaaaatggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaaatttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattaattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactagaaggattttcagctgaatcaagaaaactgcttcttatcgttcaggctcttagggacaacctggaacctgggacctttgatcttggggggctatatgaagcaattgaggagtgcctgattaatgatccctgggttttgcttaatgcttcttggttcaactccttcctcacacatgcattgagatagttgtggcaatgctactatttgctatccatactgtccaaaaaagtaccttgtttctact

SEQ ID NO: 4 - DNA, 1775 bp, synthetic construct
  source 1..1775: mol_type = other DNA
agcaaaagcaggggaaaataaaaacaaccaaaatgaaggcaaaactactggtcctgttatatgcatttgtagctacagatgcagacacaatatgtataggctaccatgcgaacaactcaaccgacactgttgacacaatactcgagaagaatgtggcagtgacacattctgttaacctgctcgaagacagccacaacgggaaactatgtaaattaaaaggaatagccccactacaattggggaaatgtaacatcaccggatggctcttgggaaatccagaatgcgactcactgcttccagcgagatcatggtcctacattgtagaaacaccaaactctgagaatggagcatgttatccaggagatctcatcgactatgaggaactgagggagcaattgagctcagtatcatcattagaaagattcgaaatatttcccaaggaaagttcatggcccaaccacacattcaacggagtaacagtatcatgctcccataggggaaaaagcagtttttacagaaatttgctatggctgacgaagaagggggattcatacccaaagctgaccaattcctatgtgaacaataaagggaaagaagtccttgtactatggggtgttcatcacccgtctagcagtgatgagcaacagagtctctatagtaatggaaatgcttatgtctctgtagcgtcttcaaattataacaggagattcaccccggaaatagctgcaaggcccaaagtaagagatcaacatgggaggatgaactattactggaccttgctagaacccggagacacaataatatttgaggcaactggtaatctaatagcaccatggtatgctttcgcactgagtagagggtttgagtccggcatcatcacctcaaacgcgtcaatgcatgagtgtaacacgaagtgtcaaacaccccagggagctataaacagcaatctccctttccagaatatacacccagtcacaataggagagtgcccaaaatatgtcaggagtaccaaattgaggatggttacaggactaagaaacatcccatccattcaatacagaggtctatttggagccattgctggttttattgaggggggatggactggaatgatagatggatggtatggttatcatcatcagaatgaacagggatcaggctatgcagcggatcaaaaaagcacacaaaatgccattaacgggattacaaacaaggtgaactctgttatcgagaaaatgaacactcaattcacagctgtgggtaaagaattcaacaacttagaaaaaaggatggaaaatttaaataaaaaagttgatgatgggtttctggacatttggacatataatgcagaattgttagttctactggaaaatgaaaggactttggatttccatgacttaaatgtgaagaatctgtacgagaaagtaaaaagccaattaaagaataatgccaaagaaatcggaaatgggtgttttgagttctaccacaagtgtgacaatgaatgcatggaaagtgtaagaaatgggacttatgattatccaaaatattcagaagaatcaaagttgaacagggaaaagatagatggagtgaaattggaatcaatgggggtgtatcagattctggcgatctactcaactgtcgccagttcactggtgcttttggtctccctgggggcaatcagtttctggatgtgttctaatgggtctttgcagtgcagaatatgcatctgagattaggatttcagaaatataaggaaaaacacccttgtttctact

SEQ ID NO: 5 - DNA, 1565 bp, synthetic construct
  source 1..1565: mol_type = other DNA
agcaaaagcagggtagataatcactcacagagtgacatcgaaatcatggcgaccaaaggcaccaaacgatcttacgaacagatggagactgatggagaacgccagaatgccactgaaatcagagcatctgtcggaaaaatgattgatggaattggacgattctacatccaaatgtgcaccgaacttaaactcagtgattatgagggacggctgattcagaacagcttaacaatagagagaatggtgctctctgcttttgacgagaggaggaataaatatctagaagaacatcccagtgcggggaaagatcctaagaaaactggaggacctatatacaggagagtagatggaaagtggaggagagaactcatcctttatgacaaagaagaaataagacgaatctggcgccaagctaataatggtgacgatgcaacggctggtctgactcacatgatgatctggcactccaatttgaatgatgcaacttaccagaggacaagagctcttgttcgcacaggaatggatcccaggatgtgctcactgatgcagggttcaaccctccctaggaggtctggggccgcaggtgctgcagtcaaaggagttggaacaatggtgatggaattgatcagaatgatcaaacgtgggatcaatgatcggaacttctggaggggtgagaatggacggagaacaaggattgcttatgaaagaatgtgcaacattctcaaagggaaatttcaaacagctgcacaaagaacaatggtggatcaagtgagagagagccggaatccaggaaatgctgagttcgaagatctcatctttttagcacggtctgcactcatattgagagggtcagttgctcacaagtcctgcctgcctgcctgtgtgtatggatctgccgtagccagtggatacgactttgaaagagagggatactctctagtcggaatagaccctttcagactgcttcaaaacagccaagtatacagcctaatcagaccaaatgagaatccagcacacaagagtcaactggtgtggatggcatgccattctgctgcatttgaagatctaagagtatcaagcttcatcagagggacgaaagtggtcccaagagggaagctttccactagaggagttcaaattgcttccaatgaaaacatggagactatggaatcaagtacccttgaactgagaagcagatactgggccataaggaccagaagtggagggaacaccaatcaacagagggcttcctcgggccaaatcagcatacaacctacgttctcagtacagagaaatctcccttttgacagaccaaccattatggcagcattcactgggaatacagaggggagaacatctgacatgagaaccgaaatcataaggctgatggaaagtgcaagaccagaagatgtgtctttccaggggcggggagtcttcgagctctcggacgaaaaggcaacgagcccgatcgtgccctcctttgacatgagtaatgaaggatcttatttcttcggagacaatgcagaggagtacgacaattaaagaaaaatacccttgtttctact

SEQ ID NO: 6 - DNA, 1409 bp, synthetic construct
  source 1..1409: mol_type = other DNA
agcgaaagcaggagtttaaatgaatccaaaccagaaaataataaccattgggtcaatctgtatggtagtcggaataattagcctaatattgcaaataggaaatataatctcaatatggattagccattcaattcaaaccggaaatcaaaaccatactggaatatgcaaccaaggcagcattacctataaagttgttgctgggcaggactcaacttcagtgatattaaccggcaattcatctctttgtcccatccgtgggtgggctatacacagcaaagacaatggcataagaattggttccaaaggagacgtttttgtcataagagagccttttatttcatgttctcacttggaatgcaggaccttttttctgactcaaggcgccttactgaatgacaagcattcaagggggacctttaaggacagaagcccttatagggccttaatgagctgccctgtcggtgaagctccgtccccgtacaattcaaggtttgaatcggttgcttggtcagcaagtgcatgtcatgatggaatgggctggctaacaatcggaatttctggtccagatgatggagcagtggctgtattaaaatacaaccgcataataactgaaaccataaaaagttggaggaagaatatattgagaacacaagagtctgaatgtacctgtgtaaatggttcatgttttaccataatgaccgatggcccaagtgatgggctggcctcgtacaaaattttcaagatcgagaaggggaaggttactaaatcgatagagttgaatgcacctaattctcactacgaggaatgttcctgttaccctgataccggcaaagtgatgtgtgtgtgcagagacaattggcacggttcgaaccgaccatgggtgtccttcgaccaaaacctagattataaaataggatacatctgcagtggggttttcggtgacaacccgcgtcccaaagatggaacaggcagctgtggcccagtgtctgctgatggagcaaacggagtaaagggattttcatataagtatggcaatggtgtttggataggaaggactaaaagtgacagttccagacatgggtttgagatgatttgggatcctaatggatggacagagactgatagtaggttctctatgagacaagatgttgtggcaataactaatcggtcagggtacagcggaagtttcgttcaacatcctgagctaacagggctagactgtatgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtagtttgttcaaaaaactccttgtttctact

SEQ ID NO: 7 - DNA, 1027 bp, synthetic construct
  source 1..1027: mol_type = other DNA
agcaaaagcaggtagatattgaaagatgagtcttctaaccgaggtcgaaacgtacgttctctctatcgtcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcggggactgcagcgtagacgctttgtccaaaatgctcttaatgggaacggagatccaaataacatggacaaagcagttaaactgtataggaagcttaagagggagataacattccatggggccaaagaaatagcactcagttattctgctggtgcacttgccagttgtatgggcctcatatacaacaggatgggggctgtgaccactgaagtggcatttggcctggtatgcgcaacctgtgaacagattgctgactcccagcatcggtctcataggcaaatggtgacaacaaccaatccactaatcagacatgagaacagaatggttctagccagcactacagctaaggctatggagcaaatggctggatcgagtgagcaagcagcagaggccatggatattgctagtcaggccaggcaaatggtgcaggcgatgagaaccgttgggactcatcctagctccagtgctggtctaaaagatgatcttcttgaaaatttgcaggcctatcagaaacgaatgggggtgcagatgcaacgattcaagtgatcctctcgtcattgcagcaaatatcattggaatcttgcacttgatattgtggattcttgatcgtctttttttcaaatgcatttatcgtcgctttaaatacggtttgaaaagagggccttctacggaaggagtgccagagtctatgagggaagaatatcgaaaggaacagcagaatgctgtggatgttgacgatggtcattttgtcaacatagagctggagtaaaaaactaccttgtttctact

SEQ ID NO: 8 - DNA, 890 bp, synthetic construct
  source 1..890: mol_type = other DNA
agcaaaagcagggtgacaaagacataatggatccaaacactgtgtcaagctttcaggtagattgctttctttggcatgtccgcaaaagagttgcagaccaagaactaggtgatgccccattccttgatcggcttcgccgagatcagaagtccctaagaggaagaggcagcactcttggtctggacatcgaaacagccacccgtgctggaaagcaaatagtggagcggattctgaaggaagaatctgatgaggcactcaaaatgaccatggcctctgtacctgcatcgcgctacctaactgacatgactcttgaggaaatgtcaaggcactggttcatgctcatgcccaagcagaaagtggcaggccctctttgtatcagaatggaccaggcgatcatggataagaacatcatactgaaagcgaacttcagtgtgatttttgaccggctggagactctaatattactaagggccttcaccgaagaggggacaattgttggcgaaatttcaccactgccctctcttccaggacatactgatgaggatgtcaaaaatgcagttggggtcctcatcggaggacttgaatggaataataacacagttcgagtctctgaaactctacagagattcgcttggagaagcagtaatgagaatgggagacctccactcactccaaaacagaaacggaaaatggcgggaacaattaggtcagaagtttgaagaaataagatggttgattgaagaagtgagacacagactgaagataacagagaatagttttgagcaaataacatttatgcaagccttacaactattgcttgaagtggagcaagagataagaactttctcgtttcagcttatttaataataaaaaacacccttgtttctact

SEQ ID NO: 9 - protein, 124 aa, synthetic construct
MAQVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGG

SEQ ID NO: 10 - protein, 131 aa, synthetic construct
MAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGDYKDDDDK

SEQ ID NO: 11 - DNA, 3094 bp, synthetic construct
  source 1..3094: mol_type = other DNA
agcgaaagcaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaacctttggccctgtccattttagaaaccaagtcaaaatacgtcgaagagttgacataaatcctggtcatgcagatctcagtgccaaagaggcacaggatgtaatcatggaagttgttttccctaacgaagtgggagccaggatactaacatcggaatcgcaactaacgacaaccaaagagaagaaagaagaactccagggttgcaaaatttctcctctgatggtggcatacatgttggagagagaactggtccgcaaaacgagattcctcccagtggctggtggaacaagcagtgtgtacattgaagtgttgcatttgacccaaggaacatgctgggaacagatgtacactccaggaggggaggcgaggaatgatgatgttgatcaaagcttaattattgctgctagaaacatagtaagaagagccacagtatcagcagatccactagcatctttattggagatgtgccacagcacgcagattggtggaataaggatggtaaacatccttaggcagaacccaacagaagagcaagccgtggatatttgcaaggctgcaatgggactgagaattagctcatccttcagttttggtggattcacatttaagagaacaagcggatcatcagtcaagagagaggaagaggtgcttacgggcaatcttcagacattgaagataagagtgcatgagggatatgaagagttcacaatggttgggagaagagcaacagctatactcagaaaagcaaccaggagattgattcagctgatagtgagtgggagagacgaacagtcgattgccgaagcaataattgtggccatggtattttcacaagaggattgtatgataaaagcagttagaggtgacctgaatttcgtcaatagggcgaatcagcgattgaatcccatgcaccaacttttgagacattttcagaaggatgcaaaggtgctctttcaaaattggggaattgaatccatcgacaatgtgatgggaatgatcgggatattgcccgacatgactccaagcaccgagatgtcaatgagaggagtgagaatcagcaaaatgggggtagatgagtattccagcgcggagaagatagtggtgagcattgaccgttttttgagagttagggaccaacgtgggaatgtactactgtctcccgaggagatcagtgaaacacagggaacagagaaactgacaataacttactcatcgtcaatgatgtgggagattaatggtcctgaatcagtgttggtcaatacctatcagtggatcatcagaaactgggaaactgttaaaattcagtggtcccagaatcctacaatgctgtacaataaaatggaatttgagccatttcagtctttagttccaaaggccgttagaggccaatacagtgggtttgtgagaactctgttccaacaaatgagggatgtgcttgggacatttgataccgctcagataataaaacttcttcccttcgcagccgctccaccaaagcaaagtagaacgcagttctcctcattgactataaatgtgaggggatcaggaatgagaatacttgtaaggggcaattctccagtattcaactacaacaagaccactaaaagactcacagttctcggaaaggatgctggccctttaactgaagacccagatgaaggcacagctggagttgagtccgcagttctgagaggattcctcattctgggcaaagaagacaggagatatggaccagcattaagcataaatgaactgagcaaccttgcgaaaggagagaaggctaatgtgctaattgggcaaggagacgtggtactagtgatgaagaggaagagaaatagctctatcttgacggattcacaaacggcaactaagaggatccgtatggctattaacggttctggcgccaccaacttctccctgctgaagcaggctggcgatgtggaggagaaccctgggcccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaagcgaaaggagagaaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 12 - DNA, 2895 bp, synthetic construct
  source 1..2895: mol_type = other DNA
atggatgtcaatccgactttacttttcttaaaagtgccagcacaaaatgctataagcacaactttcccttatactggagaccctccttacagccatgggacaggaacaggatacaccatggatactgtcaacaggacacatcagtactcagaaaggggaagatggacaacaaacaccgaaactggagcaccgcaactcaacccgattgatgggccactgccagaagacaatgaaccaagtggttatgcccaaacagattgtgtattggaagcaatggccttccttgaggaatcccatcctggtatctttgagacctcgtgtcttgaaacgatggaggttgttcagcaaacacgagtggacaagctgacacaaggccgacagacctatgactggactctaaataggaaccagcctgctgcaacagcattggccaacacaatagaagtgttcagatcaaatggcctcacggccaatgaatctggaaggctcatagacttccttaaggatgtaatggagtcaatgaacaaagaagaaatggagatcacaactcattttcagagaaagagacgagtgagagacaatatgactaagaaaatggtgacacagagaacaataggtaaaaggaagcagagattgaacaaaaggagttatctaattagggcattaaccctgaacacaatgaccaaagatgctgagagagggaagctaaaacggagagcaattgcaaccccagggatgcaaataagggggtttgtatactttgttgagacactagcaaggagtatatgtgagaaacttgaacaatcaggattgccagttggaggcaatgagaagaaagcaaagttggcaaatgttgtaaggaagatgatgaccaattctcaggacactgaaatttctttcaccatcactggagataacaccaaatggaacgaaaatcagaaccctcggatgtttttggccatgatcacatatataaccagaaatcagcccgaatggttcagaaatgttctaagtattgctccaataatgttctcaaacaaaatggcgagactgggaaaggggtacatgtttgagagcaagagtatgaaaattagaactcaaatacctgcagaaatgctagcaagcatcgatttgaaatacttcaatgattcaactagaaagaagattgaaaaaatccggccgctcttaatagatgggactgcatcattgagccctggaatgatgatgggcatgttcaatatgttaagtactgtattaggcgtctccatcctgaatcttggacaaaagagacacaccaagactacttactggtgggatggtcttcaatcttctgatgattttgctctgattgtgaatgcacccaatcatgaagggattcaagccggagtcaacaggttttatcgaacctgtaagctacttggaattaatatgagcaagaaaaagtcttacataaacagaacaggtacatttgaattcacaagttttttctatcgttatgggtttgttgccaatttcagcatggagcttcccagctttggggtgtctgggatcaacgagtctgcggacatgagtattggagttactgtcatcaaaaacaatatgataaacaatgatcttggtccagcaaccgctcaaatggcccttcagctgttcatcaaagattacaggtacacgtaccggtgccatagaggtgacacacaaatacaaacccgaagatcatttgaaataaagaaactgtgggagcaaacccattccaaagctggactgctggtctccgacggaggcccaaatttatacaacattagaaatctccacattcctgaagtctgcttgaaatgggaattaatggatgaggattaccaggggcgtttatgcaacccactgaacccatttgtcaaccataaagacattgaatcagtgaacaatgcagtgataatgccagcacatggtccagccaaaaacatggagtatgatgctgttgcaacaacacactcctggatccccaaaagaaatcgatccatcttgaatacaagccaaagaggaatacttgaagatgaacaaatgtaccaaaagtgctgcaacttatttgaaaaattcttccccagcagttcatacagaagaccagtcgggatatccagtatggtggaggctatggtttccagagcccgaattgatgcacgaattgatttcgaatctggaaggataaagaaagaggagttcactgagatcatgaaaatttgcagtacaatcgaggaacttcggagacagaagggttctggcgccaccaacttctccctgctgaagcaggctggcgatgtggaggagaaccctgggcccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 13 - DNA, 2772 bp, synthetic construct
  source 1..2772: mol_type = other DNA
atggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaaatttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattaattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactagaaggattttcagctgaatcaagaaaactgcttcttatcgttcaggctcttagggacaacctggaacctgggacctttgatcttggggggctatatgaagcaattgaggagtgcctgattaatgatccctgggttttgcttaacgccagctggtttaattcttttttgacgcacgcgctatcaggttctggcgccaccaacttctccctgctgaagcaggctggcgatgtggaggagaaccctgggcccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagttggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccttggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaa

SEQ ID NO: 14 - DNA, 3004 bp, synthetic construct
  source 1..3004: mol_type = other DNA
agcgaaagcaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaacctttggccctgtccattttagaaaccaagtcaaaatacgtcgaagagttgacataaatcctggtcatgcagatctcagtgccaaagaggcacaggatgtaatcatggaagttgttttccctaacgaagtgggagccaggatactaacatcggaatcgcaactaacgacaaccaaagagaagaaagaagaactccagggttgcaaaatttctcctctgatggtggcatacatgttggagagagaactggtccgcaaaacgagattcctcccagtggctggtggaacaagcagtgtgtacattgaagtgttgcatttgacccaaggaacatgctgggaacagatgtacactccaggaggggaggcgaggaatgatgatgttgatcaaagcttaattattgctgctagaaacatagtaagaagagccacagtatcagcagatccactagcatctttattggagatgtgccacagcacgcagattggtggaataaggatggtaaacatccttaggcagaacccaacagaagagcaagccgtggatatttgcaaggctgcaatgggactgagaattagctcatccttcagttttggtggattcacatttaagagaacaagcggatcatcagtcaagagagaggaagaggtgcttacgggcaatcttcagacattgaagataagagtgcatgagggatatgaagagttcacaatggttgggagaagagcaacagctatactcagaaaagcaaccaggagattgattcagctgatagtgagtgggagagacgaacagtcgattgccgaagcaataattgtggccatggtattttcacaagaggattgtatgataaaagcagttagaggtgacctgaatttcgtcaatagggcgaatcagcgattgaatcccatgcaccaacttttgagacattttcagaaggatgcaaaggtgctctttcaaaattggggaattgaatccatcgacaatgtgatgggaatgatcgggatattgcccgacatgactccaagcaccgagatgtcaatgagaggagtgagaatcagcaaaatgggggtagatgagtattccagcgcggagaagatagtggtgagcattgaccgttttttgagagttagggaccaacgtgggaatgtactactgtctcccgaggagatcagtgaaacacagggaacagagaaactgacaataacttactcatcgtcaatgatgtgggagattaatggtcctgaatcagtgttggtcaatacctatcagtggatcatcagaaactgggaaactgttaaaattcagtggtcccagaatcctacaatgctgtacaataaaatggaatttgagccatttcagtctttagttccaaaggccgttagaggccaatacagtgggtttgtgagaactctgttccaacaaatgagggatgtgcttgggacatttgataccgctcagataataaaacttcttcccttcgcagccgctccaccaaagcaaagtagaacgcagttctcctcattgactataaatgtgaggggatcaggaatgagaatacttgtaaggggcaattctccagtattcaactacaacaagaccactaaaagactcacagttctcggaaaggatgctggccctttaactgaagacccagatgaaggcacagctggagttgagtccgcagttctgagaggattcctcattctgggcaaagaagacaggagatatggaccagcattaagcataaatgaactgagcaaccttgcgaaaggagagaaggctaatgtgctaattgggcaaggagacgtggtactagtgatgaagaggaagagaaatagctctatcttgacggattcacaaacggcaactaagaggatccgtatggctattaacggttctggcgccaccaacttctccctgctgaagcaggctggcgatgtggaggagaaccctgggcccatggagacagacaccctgctgctgtgggtgctgctgctttgggtgcctggatctacaggagatatggcccaagtgcaactggtggaaacaggcggcggcttagtgcaacctggaggcagccttagactgagctgtacagctagcggatttaccttcagcatgcacgccatgacctggtacagacaggcccccggaaaacagagagagctggtggctgtgattaccagccacggagatagagccaactacaccgacagcgtgagaggcagattcaccatcagcagagacaacaccaagaacatggtgtacctgcagatgaacagcctgaagcccgaggacaccgccgtgtattattgcaatgtgcccagatacgacagctggggccaaggcacacaagtgacagtgagcagcggaggactgcccgaaactggaggagaacaaaaactgatcagcgaggaggacctgtaagcgaaaggagagaaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 15 - DNA, 2805 bp, synthetic construct
  source 1..2805: mol_type = other DNA
atggatgtcaatccgactttacttttcttaaaagtgccagcacaaaatgctataagcacaactttcccttatactggagaccctccttacagccatgggacaggaacaggatacaccatggatactgtcaacaggacacatcagtactcagaaaggggaagatggacaacaaacaccgaaactggagcaccgcaactcaacccgattgatgggccactgccagaagacaatgaaccaagtggttatgcccaaacagattgtgtattggaagcaatggccttccttgaggaatcccatcctggtatctttgagacctcgtgtcttgaaacgatggaggttgttcagcaaacacgagtggacaagctgacacaaggccgacagacctatgactggactctaaataggaaccagcctgctgcaacagcattggccaacacaatagaagtgttcagatcaaatggcctcacggccaatgaatctggaaggctcatagacttccttaaggatgtaatggagtcaatgaacaaagaagaaatggagatcacaactcattttcagagaaagagacgagtgagagacaatatgactaagaaaatggtgacacagagaacaataggtaaaaggaagcagagattgaacaaaaggagttatctaattagggcattaaccctgaacacaatgaccaaagatgctgagagagggaagctaaaacggagagcaattgcaaccccagggatgcaaataagggggtttgtatactttgttgagacactagcaaggagtatatgtgagaaacttgaacaatcaggattgccagttggaggcaatgagaagaaagcaaagttggcaaatgttgtaaggaagatgatgaccaattctcaggacactgaaatttctttcaccatcactggagataacaccaaatggaacgaaaatcagaaccctcggatgtttttggccatgatcacatatataaccagaaatcagcccgaatggttcagaaatgttctaagtattgctccaataatgttctcaaacaaaatggcgagactgggaaaggggtacatgtttgagagcaagagtatgaaaattagaactcaaatacctgcagaaatgctagcaagcatcgatttgaaatacttcaatgattcaactagaaagaagattgaaaaaatccggccgctcttaatagatgggactgcatcattgagccctggaatgatgatgggcatgttcaatatgttaagtactgtattaggcgtctccatcctgaatcttggacaaaagagacacaccaagactacttactggtgggatggtcttcaatcttctgatgattttgctctgattgtgaatgcacccaatcatgaagggattcaagccggagtcaacaggttttatcgaacctgtaagctacttggaattaatatgagcaagaaaaagtcttacataaacagaacaggtacatttgaattcacaagttttttctatcgttatgggtttgttgccaatttcagcatggagcttcccagctttggggtgtctgggatcaacgagtctgcggacatgagtattggagttactgtcatcaaaaacaatatgataaacaatgatcttggtccagcaaccgctcaaatggcccttcagctgttcatcaaagattacaggtacacgtaccggtgccatagaggtgacacacaaatacaaacccgaagatcatttgaaataaagaaactgtgggagcaaacccattccaaagctggactgctggtctccgacggaggcccaaatttatacaacattagaaatctccacattcctgaagtctgcttgaaatgggaattaatggatgaggattaccaggggcgtttatgcaacccactgaacccatttgtcaaccataaagacattgaatcagtgaacaatgcagtgataatgccagcacatggtccagccaaaaacatggagtatgatgctgttgcaacaacacactcctggatccccaaaagaaatcgatccatcttgaatacaagccaaagaggaatacttgaagatgaacaaatgtaccaaaagtgctgcaacttatttgaaaaattcttccccagcagttcatacagaagaccagtcgggatatccagtatggtggaggctatggtttccagagcccgaattgatgcacgaattgatttcgaatctggaaggataaagaaagaggagttcactgagatcatgaaaatttgcagtacaatcgaggaacttcggagacagaagggttctggcgccaccaacttctccctgctgaagcaggctggcgatgtggaggagaaccctgggcccatggagacagacaccctgctgctgtgggtgctgctgctttgggtgcctggatctacaggagatatggcccaagtgcaactggtggaaacaggcggcggcttagtgcaacctggaggcagccttagactgagctgtacagctagcggatttaccttcagcatgcacgccatgacctggtacagacaggcccccggaaaacagagagagctggtggctgtgattaccagccacggagatagagccaactacaccgacagcgtgagaggcagattcaccatcagcagagacaacaccaagaacatggtgtacctgcagatgaacagcctgaagcccgaggacaccgccgtgtattattgcaatgtgcccagatacgacagctggggccaaggcacacaagtgacagtgagcagcggaggactgcccgaaactggaggagaacaaaaactgatcagcgaggaggacctgtaa

SEQ ID NO: 16 - DNA, 2682 bp, synthetic construct
  source 1..2682: mol_type = other DNA
atggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaaatttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattaattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactagaaggattttcagctgaatcaagaaaactgcttcttatcgttcaggctcttagggacaacctggaacctgggacctttgatcttggggggctatatgaagcaattgaggagtgcctgattaatgatccctgggttttgcttaacgccagctggtttaattcttttttgacgcacgcgctatcaggttctggcgccaccaacttctccctgctgaagcaggctggcgatgtggaggagaaccctgggcccatggagacagacaccctgctgctgtgggtgctgctgctttgggtgcctggatctacaggagatatggcccaagtgcaactggtggaaacaggcggcggcttagtgcaacctggaggcagccttagactgagctgtacagctagcggatttaccttcagcatgcacgccatgacctggtacagacaggcccccggaaaacagagagagctggtggctgtgattaccagccacggagatagagccaactacaccgacagcgtgagaggcagattcaccatcagcagagacaacaccaagaacatggtgtacctgcagatgaacagcctgaagcccgaggacaccgccgtgtattattgcaatgtgcccagatacgacagctggggccaaggcacacaagtgacagtgagcagcggaggactgcccgaaactggaggagaacaaaaactgatcagcgaggaggacctgtaa

SEQ ID NO: 17 - DNA, 2995 bp, synthetic construct
  source 1..2995: mol_type = other DNA
agcgaaagcaggtcaattatattcaatatggaaagaataaaagaactaaggaatctaatgtcgcagtctcgcactcgcgagatactcacaaaaaccaccgtggaccatatggccataatcaagaagtacacatcaggaagacaggagaagaacccagcacttaggatgaaatggatgatggcaatgaaatatccaattacagcagacaagaggataacggaaatgattcctgagagaaatgagcagggacaaactttatggagtaaaatgaatgacgccggatcagaccgagtgatggtatcacctctggctgtgacatggtggaataggaatggaccagtgacaagtacagttcattatccaaaaatctacaaaacttattttgaaaaagtcgaaaggttaaaacatggaacctttggccctgtccattttagaaaccaagtcaaaatacgtcgaagagttgacataaatcctggtcatgcagatctcagtgccaaagaggcacaggatgtaatcatggaagttgttttccctaacgaagtgggagccaggatactaacatcggaatcgcaactaacgacaaccaaagagaagaaagaagaactccagggttgcaaaatttctcctctgatggtggcatacatgttggagagagaactggtccgcaaaacgagattcctcccagtggctggtggaacaagcagtgtgtacattgaagtgttgcatttgacccaaggaacatgctgggaacagatgtacactccaggaggggaggcgaggaatgatgatgttgatcaaagcttaattattgctgctagaaacatagtaagaagagccacagtatcagcagatccactagcatctttattggagatgtgccacagcacgcagattggtggaataaggatggtaaacatccttaggcagaacccaacagaagagcaagccgtggatatttgcaaggctgcaatgggactgagaattagctcatccttcagttttggtggattcacatttaagagaacaagcggatcatcagtcaagagagaggaagaggtgcttacgggcaatcttcagacattgaagataagagtgcatgagggatatgaagagttcacaatggttgggagaagagcaacagctatactcagaaaagcaaccaggagattgattcagctgatagtgagtgggagagacgaacagtcgattgccgaagcaataattgtggccatggtattttcacaagaggattgtatgataaaagcagttagaggtgacctgaatttcgtcaatagggcgaatcagcgattgaatcccatgcaccaacttttgagacattttcagaaggatgcaaaggtgctctttcaaaattggggaattgaatccatcgacaatgtgatgggaatgatcgggatattgcccgacatgactccaagcaccgagatgtcaatgagaggagtgagaatcagcaaaatgggggtagatgagtattccagcgcggagaagatagtggtgagcattgaccgttttttgagagttagggaccaacgtgggaatgtactactgtctcccgaggagatcagtgaaacacagggaacagagaaactgacaataacttactcatcgtcaatgatgtgggagattaatggtcctgaatcagtgttggtcaatacctatcagtggatcatcagaaactgggaaactgttaaaattcagtggtcccagaatcctacaatgctgtacaataaaatggaatttgagccatttcagtctttagttccaaaggccgttagaggccaatacagtgggtttgtgagaactctgttccaacaaatgagggatgtgcttgggacatttgataccgctcagataataaaacttcttcccttcgcagccgctccaccaaagcaaagtagaacgcagttctcctcattgactataaatgtgaggggatcaggaatgagaatacttgtaaggggcaattctccagtattcaactacaacaagaccactaaaagactcacagttctcggaaaggatgctggccctttaactgaagacccagatgaaggcacagctggagttgagtccgcagttctgagaggattcctcattctgggcaaagaagacaggagatatggaccagcattaagcataaatgaactgagcaaccttgcgaaaggagagaaggctaatgtgctaattgggcaaggagacgtggtactagtgatgaagaggaagagaaatagctctatcttgacggattcacaaacggcaactaagaggatccgtatggctattaacggttctggcgccaccaacttctccctgctgaagcaggctggcgatgtggaggagaaccctgggcccatggagacagacaccctgctgctgtgggtgctgctgctgtgggtgcctggaagcacaggagatatggctcaagtgcagctggtggagagcggcggaggactggctcaacctggaggaagccttagactgagctgtgctgctagcggaagcaccatcagcagcgtggctgtgggatggtatagacagacccccggcaatcagagagagtgggtggctacaagcagcacaagcagcacaacagccacctatgccgatagcgtgaagggaagattcaccatcagcagagacaacgccaagaacaccatctacctgcagatgaacagcctgaagcccgaggacaccgccgtttattattgcaaaaccggcctgaccaactggggcagaggcacacaagtgacagtgagcagcggaggcctgcctgaaacaggaggagattataaagatgacgatgacaagtaagcgaaaggagagaaggctaatgtgctaattgggcaaggagacgtggtgttggtaatgaaacggaaacggaactctagcatacttactgacagccagacagcgaccaaaagaattcggatggccatcaattagtgtcgaatagtttaaaaacgaccttgtttctact

SEQ ID NO: 18 - DNA, 2796 bp, synthetic construct
  source 1..2796: mol_type = other DNA
atggatgtcaatccgactttacttttcttaaaagtgccagcacaaaatgctataagcacaactttcccttatactggagaccctccttacagccatgggacaggaacaggatacaccatggatactgtcaacaggacacatcagtactcagaaaggggaagatggacaacaaacaccgaaactggagcaccgcaactcaacccgattgatgggccactgccagaagacaatgaaccaagtggttatgcccaaacagattgtgtattggaagcaatggccttccttgaggaatcccatcctggtatctttgagacctcgtgtcttgaaacgatggaggttgttcagcaaacacgagtggacaagctgacacaaggccgacagacctatgactggactctaaataggaaccagcctgctgcaacagcattggccaacacaatagaagtgttcagatcaaatggcctcacggccaatgaatctggaaggctcatagacttccttaaggatgtaatggagtcaatgaacaaagaagaaatggagatcacaactcattttcagagaaagagacgagtgagagacaatatgactaagaaaatggtgacacagagaacaataggtaaaaggaagcagagattgaacaaaaggagttatctaattagggcattaaccctgaacacaatgaccaaagatgctgagagagggaagctaaaacggagagcaattgcaaccccagggatgcaaataagggggtttgtatactttgttgagacactagcaaggagtatatgtgagaaacttgaacaatcaggattgccagttggaggcaatgagaagaaagcaaagttggcaaatgttgtaaggaagatgatgaccaattctcaggacactgaaatttctttcaccatcactggagataacaccaaatggaacgaaaatcagaaccctcggatgtttttggccatgatcacatatataaccagaaatcagcccgaatggttcagaaatgttctaagtattgctccaataatgttctcaaacaaaatggcgagactgggaaaggggtacatgtttgagagcaagagtatgaaaattagaactcaaatacctgcagaaatgctagcaagcatcgatttgaaatacttcaatgattcaactagaaagaagattgaaaaaatccggccgctcttaatagatgggactgcatcattgagccctggaatgatgatgggcatgttcaatatgttaagtactgtattaggcgtctccatcctgaatcttggacaaaagagacacaccaagactacttactggtgggatggtcttcaatcttctgatgattttgctctgattgtgaatgcacccaatcatgaagggattcaagccggagtcaacaggttttatcgaacctgtaagctacttggaattaatatgagcaagaaaaagtcttacataaacagaacaggtacatttgaattcacaagttttttctatcgttatgggtttgttgccaatttcagcatggagcttcccagctttggggtgtctgggatcaacgagtctgcggacatgagtattggagttactgtcatcaaaaacaatatgataaacaatgatcttggtccagcaaccgctcaaatggcccttcagctgttcatcaaagattacaggtacacgtaccggtgccatagaggtgacacacaaatacaaacccgaagatcatttgaaataaagaaactgtgggagcaaacccattccaaagctggactgctggtctccgacggaggcccaaatttatacaacattagaaatctccacattcctgaagtctgcttgaaatgggaattaatggatgaggattaccaggggcgtttatgcaacccactgaacccatttgtcaaccataaagacattgaatcagtgaacaatgcagtgataatgccagcacatggtccagccaaaaacatggagtatgatgctgttgcaacaacacactcctggatccccaaaagaaatcgatccatcttgaatacaagccaaagaggaatacttgaagatgaacaaatgtaccaaaagtgctgcaacttatttgaaaaattcttccccagcagttcatacagaagaccagtcgggatatccagtatggtggaggctatggtttccagagcccgaattgatgcacgaattgatttcgaatctggaaggataaagaaagaggagttcactgagatcatgaaaatttgcagtacaatcgaggaacttcggagacagaagggttctggcgccaccaacttctccctgctgaagcaggctggcgatgtggaggagaaccctgggcccatggagacagacaccctgctgctgtgggtgctgctgctgtgggtgcctggaagcacaggagatatggctcaagtgcagctggtggagagcggcggaggactggctcaacctggaggaagccttagactgagctgtgctgctagcggaagcaccatcagcagcgtggctgtgggatggtatagacagacccccggcaatcagagagagtgggtggctacaagcagcacaagcagcacaacagccacctatgccgatagcgtgaagggaagattcaccatcagcagagacaacgccaagaacaccatctacctgcagatgaacagcctgaagcccgaggacaccgccgtttattattgcaaaaccggcctgaccaactggggcagaggcacacaagtgacagtgagcagcggaggcctgcctgaaacaggaggagattataaagatgacgatgacaagtaa

SEQ ID NO: 19 - DNA, 2673 bp, synthetic construct
  source 1..2673: mol_type = other DNA
atggaagattttgtgcgacaatgcttcaatccgatgattgtcgagcttgcggaaaaggcaatgaaagagtatggagaggacctgaaaatcgaaacaaacaaatttgcagcaatatgcactcacttggaagtgtgcttcatgtattcagattttcacttcatcgatgagcaaggcgagtcaatagtcgtagaacttggcgatccaaatgcacttttgaagcacagatttgaaataatcgagggaagagatcgcacaatagcctggacagtaataaacagtatttgcaacactacaggggctgagaaaccaaagtttctaccagatttgtatgattacaagaagaatagattcatcgaaattggagtaacaaggagagaagttcacatatactatctggaaaaggccaataaaattaaatctgagaagacacacatccacattttctcattcactggggaggaaatggccacaaaggccgactacactctcgatgaagaaagcagggctaggatcaaaaccaggctattcaccataagacaagaaatggctagcagaggcctctgggattcctttcgtcagtccgagagaggcgaagagacaattgaagaaagatttgaaatcacaggaacaatgcgcaagcttgccgaccaaagtctcccgccaaacttctccagccttgaaaaatttagagcctatgtggatggattcgaaccgaacggctacattgagggcaagctttctcaaatgtccaaagaagtaaatgctagaattgaaccttttttgaaatcaacaccacgaccacttagacttccggatgggcctccctgttctcagcggtccaaattcctgctgatggatgccttaaaattaagcattgaggacccaagtcatgagggagaggggataccgctatatgatgcaatcaaatgcatgagaacattctttggatggaaggaacccaatgttgttaaaccacacgaaaagggaataaatccaaattatcttctgtcatggaagcaagtactggcagaactgcaggacattgagaatgaggagaaaattccaaggactaaaaatatgaagaaaacgagtcagttaaagtgggcacttggtgagaacatggcaccagaaaaggtagactttgacgattgtaaagatgtaggcgatttgaagcaatatgatagtgatgaaccagaattgaggtcgcttgcaagttggattcagaatgagttcaacaaggcatgtgaactgaccgattcaagctggatagagctcgatgagattggagaagatgcggctccaattgaacacattgcaagcatgagaaggaattatttcacagcagaggtgtctcattgcagagccacagaatacataatgaagggggtgtacatcaatactgccttgcttaatgcatcctgtgcagcaatggatgatttccaattaattccaatgataagcaagtgtagaactaaggagggaaggcgaaagaccaatttgtacggtttcatcataaaaggaagatcccacttaaggaatgacaccgatgtggtaaactttgtgagcatggagttttccctcactgacccaagacttgaaccacacaaatgggagaagtactgtgttcttgaggtaggagatatgcttctaagaagtgccataggccatgtgtcaaggcctatgttcttgtatgtgaggacaaatggaacctcaaaaattaaaatgaaatgggggatggaaatgaggcgttgcctccttcagtcacttcaacaaatcgagagtatgattgaagctgagtcctctgtcaaggagaaagacatgaccaaagagttctttgaaaacaaatcagaaacatggcccgttggagagtcccccaaaggagtggaggaaggttccattgggaaggtctgcagaactttattggcaaagtcggtattcaacagcttgtatgcatctccacaactagaaggattttcagctgaatcaagaaaactgcttcttatcgttcaggctcttagggacaacctggaacctgggacctttgatcttggggggctatatgaagcaattgaggagtgcctgattaatgatccctgggttttgcttaacgccagctggtttaattcttttttgacgcacgcgctatcaggttctggcgccaccaacttctccctgctgaagcaggctggcgatgtggaggagaaccctgggcccatggagacagacaccctgctgctgtgggtgctgctgctgtgggtgcctggaagcacaggagatatggctcaagtgcagctggtggagagcggcggaggactggctcaacctggaggaagccttagactgagctgtgctgctagcggaagcaccatcagcagcgtggctgtgggatggtatagacagacccccggcaatcagagagagtgggtggctacaagcagcacaagcagcacaacagccacctatgccgatagcgtgaagggaagattcaccatcagcagagacaacgccaagaacaccatctacctgcagatgaacagcctgaagcccgaggacaccgccgtttattattgcaaaaccggcctgaccaactggggcagaggcacacaagtgacagtgagcagcggaggcctgcctgaaacaggaggagattataaagatgacgatgacaagtaa

SEQ ID NO: 20 - DNA, 20 bp, synthetic construct
  source 1..20: mol_type = other DNA
tccatgacgttcctgacgtt

SEQ ID NO: 21 - DNA, 22 bp, synthetic construct
  source 1..22: mol_type = other DNA
aataacccggcggcccaaaatg

SEQ ID NO: 22 - DNA, 18 bp, synthetic construct
  source 1..18: mol_type = other DNA
cccccccaacttcggagg

SEQ ID NO: 23 - DNA, 44 bp, synthetic construct
  source 1..44: mol_type = other DNA
gacctccgaagttgggggggagcgaaagcaggtcaattatattc

SEQ ID NO: 24 - DNA, 45 bp, synthetic construct
  source 1..45: mol_type = other DNA
ttttgggccgccgggttattagtagaaacaaggtcgtttttaaac

SEQ ID NO: 25 - DNA, 38 bp, synthetic construct
  source 1..38: mol_type = other DNA
gacctccgaagttgggggggagcgaaagcaggcaaacc

SEQ ID NO: 26 - DNA, 48 bp, synthetic construct
  source 1..48: mol_type = other DNA
ttttgggccgccgggttattagtagaaacaaggcattttttcatgaag

SEQ ID NO: 27 - DNA, 41 bp, synthetic construct
  source 1..41: mol_type = other DNA
gacctccgaagttgggggggagcgaaagcaggtactgattc

SEQ ID NO: 28 - DNA, 44 bp, synthetic construct
  source 1..44: mol_type = other DNA
ttttgggccgccgggttattagtagaaacaaggtacttttttgg

SEQ ID NO: 29 - DNA, 24 bp, synthetic construct
  source 1..24: mol_type = other DNA
gcgaaaggagagaaggctaatgtg

SEQ ID NO: 30 - DNA, 18 bp, synthetic construct
  source 1..18: mol_type = other DNA
gggcccagggttctcctc

SEQ ID NO: 31 - DNA, 38 bp, synthetic construct
  source 1..38: mol_type = other DNA
tggaggagaaccctgggcccatggagacagacaccctg

SEQ ID NO: 32 - DNA, 39 bp, synthetic construct
  source 1..39: mol_type = other DNA
ttagccttctctcctttcgcttacaggtcctcctcgctg

SEQ ID NO: 33 - DNA, 23 bp, synthetic construct
  source 1..23: mol_type = other DNA
taatccagagcccgaattgatgc

SEQ ID NO: 34 - DNA, 18 bp, synthetic construct
  source 1..18: mol_type = other DNA
gggcccagggttctcctc

SEQ ID NO: 35 - DNA, 38 bp, synthetic construct; identical to SEQ ID NO: 31
  source 1..38: mol_type = other DNA

SEQ ID NO: 36 - DNA, 39 bp, synthetic construct
  source 1..39: mol_type = other DNA
gcatcaattcgggctctggattacaggtcctcctcgctg

SEQ ID NO: 37 - DNA, 26 bp, synthetic construct
  source 1..26: mol_type = other DNA
taagaacctgggacctttgatcttgg

SEQ ID NO: 38 - DNA, 18 bp, synthetic construct
  source 1..18: mol_type = other DNA
gggcccagggttctcctc

SEQ ID NO: 39 - DNA, 38 bp, synthetic construct; identical to SEQ ID NO: 31
  source 1..38: mol_type = other DNA

SEQ ID NO: 40 - DNA, 39 bp, synthetic construct
  source 1..39: mol_type = other DNA
agatcaaaggtcccaggttcttacaggtcctcctcgctg

SEQ ID NO: 41 - DNA, 38 bp, synthetic construct; identical to SEQ ID NO: 31
  source 1..38: mol_type = other DNA

SEQ ID NO: 42 - DNA, 47 bp, synthetic construct
  source 1..47: mol_type = other DNA
ttagccttctctcctttcgcttacttgtcatcgtcatctttataatc

SEQ ID NO: 43 - DNA, 38 bp, synthetic construct; identical to SEQ ID NO: 31
  source 1..38: mol_type = other DNA

SEQ ID NO: 44 - DNA, 46 bp, synthetic construct
  source 1..46: mol_type = other DNA
tcaattcgggctctggattacttgtcatcgtcatctttataatctc

SEQ ID NO: 45 - DNA, 38 bp, synthetic construct; identical to SEQ ID NO: 31
  source 1..38: mol_type = other DNA

SEQ ID NO: 46 - DNA, 46 bp, synthetic construct
  source 1..46: mol_type = other DNA
tcaaaggtcccaggttcttacttgtcatcgtcatctttataatctc

SEQ ID NO: 47 - protein, 23 aa, synthetic construct
SIINFEKLTEWTSSNVMEERKIK

SEQ ID NO: 48 - protein, 17 aa, synthetic construct
ISQAVHAAHAEINEAGR

SEQ ID NO: 49 - protein, 20 aa, synthetic construct
AVGALEGPRNQDWLGVPRQL

SEQ ID NO: 50 - protein, 20 aa, synthetic construct
EGPAFLTWHRYHLLQLERDM

SEQ ID NO: 51 - protein, 20 aa, synthetic construct
QIANCSVYDFFVWLHYYSVR

SEQ ID NO: 52 - protein, 27 aa, synthetic construct
VTSIPSVSNALNWKEFSFIQSTLGYVA

SEQ ID NO: 53 - protein, 27 aa, synthetic construct
EGDPCLRSSDCIDEFCCARHFWTKICK

SEQ ID NO: 54 - protein, 27 aa, synthetic construct
PLLPFYPPDEALEIGLELNSSALPPTE

SEQ ID NO: 55 - protein, 27 aa, synthetic construct
SHRSCSHQTSAPSPKALAHNGTPRNAI

SEQ ID NO: 56 - DNA, 12 bp, synthetic construct
  source 1..12: mol_type = other DNA
agcgaaagcagg

SEQ ID NO: 57 - DNA, 24 bp, synthetic construct
  source 1..24: mol_type = other DNA
agcgaaagcaggtcaattatattc

SEQ ID NO: 58 - DNA, 25 bp, synthetic construct
  source 1..25: mol_type = other DNA
ggtcgtttttaaactattcgacact

(19) United States
(12) Patent Application Publication
(10) Pub. No.: US 2026/0061044 A1
(43) Pub. Date: Mar. 5, 2026

(54) Mutated Influenza Virus, Pharmaceutical Composition, and Use

(71) Applicants: NINGBO INSTITUTE OF MARINE MEDICINE, PEKING UNIVERSITY, Zhejiang (CN); PEKING UNIVERSITY, Beijing (CN)

(72) Inventors: Demin ZHOU, Beijing (CN); Dezhong JI, Beijing (CN); Yuanjie ZHANG, Beijing (CN)

(21) Appl. No.: 19/106,096

(22) PCT Filed: Aug. 24, 2022
(86) PCT No.: PCT/CN2022/114416

§ 371 Date: Feb. 24, 2025

(51) Int. Cl. A61K 39/145; A61K 39/00; A61P 35/00; A61P 37/04; C07K 14/005; C07K 16/28; C12N 15/86

(52) U.S. Cl. CPC A61K 39/145; A61P 35/00; A61P 37/04; C07K 14/005; C07K 16/2818; C07K 16/2827; C12N 15/86; A61K 2039/5256; A61K 2039/585; C07K 2317/569; C12N 2760/16121; C12N 2760/16122; C12N 2760/16134; C12N 2760/16143

(57) ABSTRACT

The present invention belongs to the field of medicines and relates to a mutated influenza virus, a pharmaceutical composition, and use. Specifically, the present invention relates to a replication-defective influenza virus, a pharmaceutical composition, and use. More specifically, the present invention relates to a mutated influenza virus, wherein nucleic acid encoding HA protein and/or nucleic acid encoding NA protein of the influenza virus comprises one or more UAG codons. The replication-defective influenza virus or the pharmaceutical composition of the present invention can effectively treat or prevent tumors and has good application prospects.

### TECHNICAL FIELD

The present invention belongs to the field of medicine, and relates to a mutated influenza virus, a pharmaceutical composition, and use thereof. Specifically, the present invention relates to a replication-deficient influenza virus, a pharmaceutical composition, and use thereof.

### BACKGROUND ART

Influenza virus is a segmented single-stranded negative-strand RNA virus, which belongs to the Orthomyxovoridae family and the influenza virus genus in virus taxonomy. Based on the difference in the antigenicity of nucleoprotein (NP) and matrix protein (M) inside the virus, influenza viruses can be divided into types A, B, C and D, among which type A influenza virus can infect humans, pigs, horses and poultry; type B influenza virus only infects humans; type C influenza virus mainly infects humans and pigs; and type D influenza virus is a newly discovered bovine influenza virus. Type A influenza virus contains 8 single-stranded negative-strand RNA segments, encoding a total of 11 proteins: 3 polymerase proteins PB2, PB1 and PA, nucleoprotein NP, hemagglutinin HA, neuraminidase NA, matrix protein M1 and ion channel M2 encoded by M gene, non-structural proteins NS1, NS2 and PB1-F2 encoded by NS gene. Influenza virus can be divided into different subtypes according to the difference in antigenicity of surface structural proteins HA and NA. Type A influenza virus has 18 different hemagglutinin subtypes (H1 to H18) and 11 neuraminidase subtypes (N1 to N11).

Influenza virus is usually spherical with a diameter of 80 to 120 nm. There are two glycoproteins HA and NA distributed on the surface of virus particles, they have different shapes and are both antigenic, and the ratio of HA to NA is (4-5):1. There is also M2 ion channel transmembrane protein on the surface of influenza virus particles, and the ratio of M2 to HA is 1:(10-100). Matrix layer is located under the viral envelope, and composed of matrix protein M1, which has the highest content in the virus particles and constitutes the skeleton of the viral envelope. Ribonucleoprotein (RNP) complex and non-structural protein 2 (also known as nuclear export protein NEP) are located inside the matrix layer and connected to the M1 protein, in which the ribonucleoprotein (RNP) complex is composed of 4 proteins and RNA: three polymerase proteins (PB1, PB2 and PA) form an RNA-dependent RNA polymerase complex, while the nucleoprotein (NP) surrounds the viral RNA genome.

Reverse genetics is to generate viruses from full-length cDNA copies of viral genomes and is one of the most powerful genetic tools in modern virology. At present, the de novo synthesis of influenza viruses is mainly achieved by co-transfecting cells with 8 to 12 plasmids. Among them, the 12-plasmid system is the most widely used to rescue influenza A viruses; 8 plasmids each contain a promoter and terminator that are recognizable by RNA polymerase I, and cDNA of influenza virus segmented genome between the promoter and terminator, while the other 4 plasmids express four proteins (PA, PB1, PB2 and NP).

Gene codon expansion technology is relative to the normal protein translation system. There are a total of 64 triplet codons in nature. In general organisms, 61 of them encode 20 natural amino acids, while the other three codons (UAA, UGA, UAG) do not encode any amino acids. When ribosome translates these codons, there will be normal termination factors to terminate protein translation. Under normal circumstances, each amino acid in the living body has a matching aminoacyl tRNA synthetase and tRNA, which is a matching system. The matching systems for two amino acids cannot be used interchangeably. For example, glycine will not be recognized and utilized by the glutamate system, and conversely, glutamate will not be recognized and utilized by the glycine system. Each system exists independently of each other and is extremely rigorous. Therefore, there are 20 systems corresponding to 20 amino acids in the living body. Gene codon expansion technology is to independently add a new system into the living body, making it a set of 21 systems. In the 21st system, the bio-orthogonal aminoacyl-tRNA synthetase and tRNA recognize the amber stop codon and artificially designed and synthesized non-natural amino acid (e.g., NAEK, etc.), and insert the non-natural amino acid into the position of the amber stop codon, which is equivalent to changing the gene codons from the original 61 to 62 codons, that is, there is an additional unnatural amino acid that can be recognized, thereby expanding the gene codons of organisms. The amber stop codon can be introduced into any position of the wild-type virus genome through site-directed mutagenesis, and a premature termination codon-harbouring virus (PTC virus) can be designed. The PTC virus can perform normal translation of viral proteins in the presence of non-natural amino acids in cells containing bio-orthogonal aminoacyl-tRNA synthetase and tRNA, and then be replicated; therefore, the 21^{st} bio-orthogonal system can be used for large-scale preparation of PTC virus. However, in normal cells, there are no bio-orthogonal aminoacyl-tRNA synthetase and tRNA, nor non-natural amino acids, so the virus can only infect normal cells like wild-type viruses, and cannot be replicated after infection, and thus is safe for normal cells. Therefore, PTC virus can be developed into a replication-controlled live influenza virus vaccine.

Tumor vaccines aim to use to produce antigens produced by tumor cell mutations and are different from autologous cells, and use vacination to enhance the immune system's recognition of tumor antigens, thereby identifying and eliminating tumor cells. In the past 30 years, tumor vaccines have mainly focused on two aspects of research: 1. Identification of tumor-associated antigens and tumor neoantigens; 2. Research on vaccine vectors, that is, research based on existing tumor antigens to study how to deliver identified tumor antigens and tumor immune responses against the tumor antigens, which are mainly divided into the following categories: (1) tumor cell vaccines, i.e., full tumor antigens that are obtained by separating tumor cells and artificially inactivating them, and then used to immunize patients; the advantage of this method is that it is simple to prepare, but the disadvantage is that the immune response as activated is weak; (2) gene vaccines, i.e., specific tumor antigens that are artificially encoded by using gene therapy vectors such as lentivirus and adenovirus; the advantage of this method is that it can selectively select antigens with strong immunogenicity and specificity for immunization, but the disadvantage is that specific gene therapy vectors are required, and the stimulation of the body's immune response is usually weak; (3) peptide vaccines, i.e., specific amino acid sequences that are artificially synthesized for immunization based on the predicted tumor antigen epitope amino acid sequences, which activate immune cells in vivo through the presentation of dendritic cells to kill tumors; its advantage is that it can be designed for specific tumor antigens, and the cost of chemically synthesized peptides is low, but the disadvantage is that its immunogenicity is poor; (4) dendritic cell vaccines, i.e., specific antigen presenting cells (dendritic cells) in human body, which are used to carry tumor antigens through in vitro culture, and activate immune system by in vivo infusion; the advantage is that dendritic cells are autologous cells and the most important antigen presenting cells with the strongest T cell activation ability, but the disadvantage is that dendritic cells are obtained through a method similar to CAR-T preparation, which requires in vitro culture of dendritic cells, which is extremely costly and has harsh in vitro culture conditions and is not easy to scale up.

From the above technologies, it can be seen that the basis of tumor vaccine research and development is tumor antigen peptides. Tumor antigen peptides are a type of artificially synthesized peptides with a length of about 20 amino acids that contain T cell or B cell epitopes. Since the peptides have a small molecular weight and limited immunogenicity, and the basis of tumor immunity is to produce a strong immune response, the research of peptide-based tumor vaccines in the past few decades has mainly focused on how to improve the immunogenicity of peptides.

Immune checkpoint inhibitors are a new type of tumor immunotherapy with significant clinical efficacy, but the systemic toxicity caused by their systemic application cannot be ignored. How to deliver immune checkpoint inhibitors specifically to tumor tissues and avoid systemic toxicity is a key point of the research and development in this field.

In recent years, with the continuous deepening of tumor immune microenvironment research, the concepts of “cold” and “hot” tumor microenvironments have been proposed. “Cold” tumors are a type of tumors with low immune cell infiltration and poor response to immunotherapy, which is currently difficult to treat clinically. Therefore, reversing the “cold” tumor microenvironment can greatly improve the effect of current tumor immunotherapy and is one of the main development directions of tumor immunotherapy.

As far as the inventors know, there has been no invention that reports using a replication-defective virus as a tumor antigen peptide carrier to prepare tumor vaccines.

### Contents of the Present Invention

After in-depth research and creative work, the inventors used a replication-deficient influenza virus as a carrier, coupled a tumor antigen peptide to an influenza virus envelope protein (hemagglutinin) through non-natural amino acids, modified with an immune adjuvant CpG on the surface of influenza virus envelope through membrane insertion technology, and inserted a gene expressing immune checkpoint inhibitor through influenza virus gene modification, which combined the advantages of tumor antigen peptide vaccine+immune checkpoint inhibitor+tumor microenvironment regulator+active targeting, and solved the following problems existing in existing tumor immunotherapy: 1) low immunogenicity of tumor antigen peptide alone; 2) systemic toxicity of immune checkpoint inhibitor as systemically applied; 3) reduction of efficacy of tumor immunotherapy due to tumor suppressive immune microenvironment. Therefore, the following invention is provided:

One aspect of the present invention relates to a mutated influenza virus, which comprises a nucleic acid encoding HA protein and/or a nucleic acid encoding NA protein, wherein the nucleic acid encoding HA protein and/or the nucleic acid encoding NA protein contains one or more (e.g., 2-10, 2-5, 2, 3, 4 or 5) UAG codons.

The mutated influenza virus is an artificially mutated influenza virus.

In some embodiments of the present invention, the mutated influenza virus is provided, wherein the nucleic acid comprises a codon mutated to UAG, wherein the codon encoding a site of HA protein selected from one or more of the following:
C84, S86, S92, S126, E132, P135, G147, K170, K176, N179, S201, 1256, S53, K57, K62, 164, A65, L67, K71 or P82.

In some embodiments of the present invention, the mutated influenza virus is provided, which further comprises a nucleic acid encoding PB1 protein, PA protein and/or NP protein, wherein the nucleic acid encoding PB1 protein, PA protein and/or NP protein contains one or more (e.g., 2 to 10, 2 to 5, 2, 3, 4 or 5) UAG codons or TAG codons.

In some embodiments of the present invention, the mutated influenza virus is provided, wherein the nucleic acid codon encoding the following site is mutated to UAG codon:
R52 site of PB1 protein,
R266 site of PA protein,
and/or
D101 site of NP protein.

In some embodiments of the present invention, the mutated influenza virus is provided, wherein the nucleic acid codons encoding the following sites are mutated to UAG codons:
R52 site of PB1 protein, R266 site of PA protein, S53 site of HA protein and D101 site of NP protein;
K33 site of PB2 protein, R266 site of PA protein, S53 site of HA protein and D101 site of NP protein;
or
K33 site of PB2 protein, R52 site of PB1 protein, S53 site of HA protein and D101 site of NP protein.

In some embodiments of the present invention, the mutated influenza virus is provided, wherein before mutation the influenza virus is a wild-type influenza virus; preferably, a wild-type influenza virus A/WSN/1933.

In some embodiments of the present invention, the mutated influenza virus is provided, wherein:
the amino acid sequence of the PB2 protein unmutated is the same as the amino acid sequence encoded by SEQ ID NO: 1;
the amino acid sequence of the PB1 protein unmutated is the same as the amino acid sequence encoded by SEQ ID NO: 2;
the amino acid sequence of the PA protein unmutated is the same as the amino acid sequence encoded by SEQ ID NO: 3;
the amino acid sequence of the HA protein unmutated is the same as the amino acid sequence encoded by SEQ ID NO: 4;
the amino acid sequence of the NP protein unmutated is the same as the amino acid sequence encoded by SEQ ID NO: 5;
the amino acid sequence of the NA protein unmutated is the same as the amino acid sequence encoded by SEQ ID NO: 6;
the amino acid sequence of the M protein unmutated is the same as the amino acid sequence encoded by SEQ ID NO: 7;
and/or
the amino acid sequence of the NS protein unmutated is the same as the amino acid sequence encoded by SEQ ID NO: 8.

In some embodiments of the present invention, the mutated influenza virus is provided, wherein one or more of the UAG codons are located upstream of stop codon.

In some embodiments of the present invention, the mutated influenza virus is provided, wherein the amino acids at the positions encoded by one or more of the UAG codons are the same or different non-natural amino acids, such as NAEK.

The present invention also relates to a mutated influenza virus, wherein the following sites are mutated to non-natural amino acids such as NAEK:
R52 site of PB1 protein, R266 site of PA protein, S53 site of HA protein and D101 site of NP protein;
K33 site of PB2 protein, R266 site of PA protein, S53 site of HA protein and D101 site of NP protein;
or
K33 site of PB2 protein, R52 site of PB1 protein, S53 site of HA protein and D101 site of NP protein.

In another aspect, the present invention relates to a recombinant influenza virus, which is obtainable by recombining the mutated influenza virus described according to any one of the items of the present invention with an exogenous nucleic acid;
preferably, the exogenous nucleic acid is inserted into the nucleic acid encoding PB2, PB1 or PA;
preferably, the exogenous nucleic acid is a nucleic acid encoding an anti-tumor antibody; preferably, the anti-tumor antibody is an anti-PD-1 antibody, an anti-PD-L1 antibody and/or an anti-CTLA-4 antibody;
preferably, the anti-tumor antibody is a nanobody, a single-chain antibody, a monoclonal antibody or a bispecific antibody;
preferably, the anti-tumor antibody is an anti-PD-1 nanobody or an anti-CTLA-4 nanobody;
preferably, the amino acid sequence of the anti-PD-1 nanobody is as shown in SEQ ID NO: 9;
preferably, the amino acid sequence of the anti-CTLA-4 nanobody is as shown in SEQ ID NO: 10.

The amino acid sequence of the anti-PD-1 nanobody 5dxw is:

(SEQ ID NO: 9)
MAQVQLVETGGGLVQPGGSLRLSCTASGFTFSMHAMTWYRQAPGKQRELV
AVITSHGDRANYTDSVRGRFTISRDNTKNMVYLQMNSLKPEDTAVYYCNV
PRYDSWGQGTQVTVSSGGLPETGG

The amino acid sequence of the anti-CTLA-4 nanobody 5e03 is:

(SEQ ID NO: 10)
MAQVQLVESGGGLAQPGGSLRLSCAASGSTISSVAVGWYRQTPGNQREWV
ATSSTSSTTATYADSVKGRFTISRDNAKNTIYLQMNSLKPEDTAVYYCKT
GLTNWGRGTQVTVSSGGLPETGGDYKDDDDK

Another aspect of the present invention relates to a modified influenza virus, wherein one or more anti-tumor antigen peptides are attached to the surface of the mutated influenza virus or the recombinant influenza virus as described in any one of the items of the present invention.

In some embodiments of the present invention, the modified influenza virus is provided, wherein the antigen peptide is linked to the hemagglutinin protein (HA) or NA on the surface of the virus; preferably, the antigen peptide is linked to a non-natural amino acid such as NAEK in the HA protein or NA protein.

In some embodiments of the present invention, the modified influenza virus is provided, wherein the antigen peptide is attached to the non-natural amino acid in the HA protein in the form of any compound selected from the compounds as shown in Formula III-1 to Formula III-6:

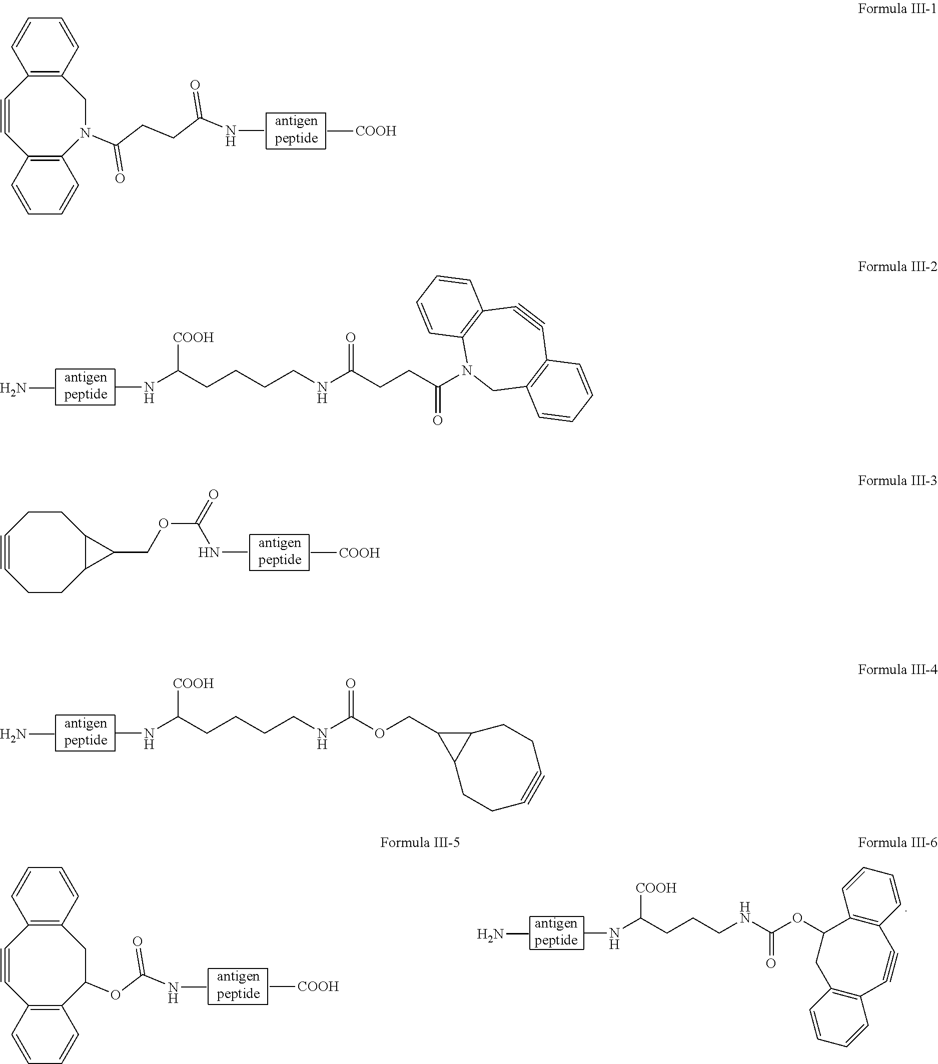

In some embodiments of the present invention, the modified influenza virus is provided, wherein the amino acid sequence of the antigen peptide is independently selected from of any one of sequences as shown in SEQ ID NOs: 49-55.

The click reaction is an efficient, rapid, orthogonal reaction between azide and an alkyne bond-containing compound. In the present invention, an alkyne bond-containing group is modified on an exogenous antigen peptide, which can react with an azide group on virus surface hemagglutinin protein (HA), thereby achieving a covalent modification.

In some embodiments of the present invention, the modified influenza virus is provided, wherein 100 to 500 antigen peptides, preferably 200 to 400 antigen peptides, more preferably 250 to 350 antigen peptides or 300 antigen peptides are coupled to one virus particle.

In some embodiments of the present invention, the modified influenza virus is provided, wherein a CpG adjuvant is further attached to the surface of the modified influenza virus;
preferably, the CpG adjuvant has a sequence as shown in SEQ ID NO: 20 (TCCATGACGTTCCTGACGTT);
preferably, the CpG adjuvant is attached to the virus surface in the form of a compound as shown in Formula IV,

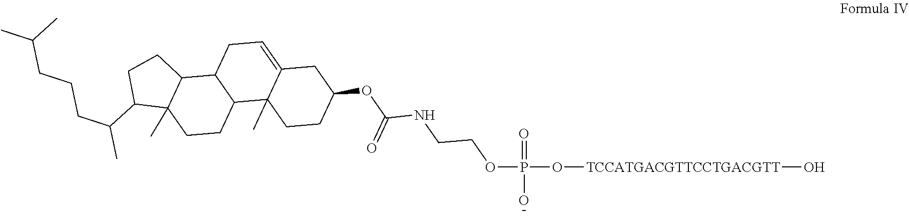

wherein, “TCCATGACGTTCCTGACGTT” (SEQ ID NO: 20) in Formula IV is the CpG adjuvant.

Another aspect of the present invention relates to a pharmaceutical composition, which comprises the mutated influenza virus described in any one of the items of the present invention, the recombinant influenza virus described in any one of the items of the present invention, and/or the modified influenza virus described in any one of the items of the present invention;
optionally, the pharmaceutical composition further comprises one or more pharmaceutically acceptable excipients;
preferably, the pharmaceutical composition is a vaccine composition;
preferably, the vaccine composition is an anti-tumor vaccine composition;
preferably, the tumor is selected from the group consisting of primary lung tumor and lung metastasis tumor;
preferably, the tumor is one or more selected from the group consisting of lung cancer (small cell lung cancer or non-small cell lung cancer), lung metastasis from melanoma, lung metastasis from breast cancer, and lung metastasis from colon cancer.

Another aspect of the present invention relates to a use of the mutated influenza virus described in any one of the items of the present invention, the recombinant influenza virus described in any one of the items of the present invention, and/or the modified influenza virus described in any one of the items of the present invention in the manufacture of a medicament for treating or preventing a tumor;
preferably, the tumor is selected from the group consisting of primary lung tumor and lung metastasis tumor;
preferably, the tumor is one or more selected from the group consisting of lung cancer (small cell lung cancer or non-small cell lung cancer), lung metastasis from melanoma, lung metastasis from breast cancer, and lung metastasis from colon cancer.

Further another aspect of the present invention relates to a method for treating or preventing a tumor, comprising a step of administering to a subject in need thereof an effective amount of the mutated influenza virus described in any one of the items of the present invention, the recombinant influenza virus described in any one of the items of the present invention, and/or the modified influenza virus described in any one of the items of the present invention;
preferably, the tumor is selected from the group consisting of primary lung tumor and lung metastasis tumor;
preferably, the tumor is one or more selected from the group consisting of lung cancer (small cell lung cancer or non-small cell lung cancer), lung metastasis from melanoma, lung metastasis from breast cancer, and lung metastasis from colon cancer.

The mutated influenza virus described in any one of the items of the present invention, the recombinant influenza virus described in any one of the items of the present invention, and/or the modified influenza virus described in any one of the items of the present invention, is used for treating or preventing a tumor;
preferably, the tumor is selected from the group consisting of primary lung tumor and lung metastasis tumor;
preferably, the tumor is one or more selected from the group consisting of lung cancer (small cell lung cancer or non-small cell lung cancer), lung metastasis from melanoma, lung metastasis from breast cancer, and lung metastasis from colon cancer.

Further another aspect of the present invention relates to a plasmid-based influenza virus reverse genetics system, comprising:
(1) 8 recombinant plasmids containing PB2 gene, PB1 gene, PA gene, HA gene, NP gene, NA gene, M gene and NS gene, respectively, and a vector, and
(2) 4 additional recombinant plasmids containing PB2 gene, PB1 gene, PA gene and NP gene, respectively, and a vector;
the vector used for the recombinant plasmid in (2) is different from the vector used for the recombinant plasmid in (1); and the PB2 gene, PB1 gene, PA gene and NP gene in (2) are wild type;
wherein the HA gene and/or NA gene contain one or more (e.g., 2, 3, 4 or 5) TAG codons.

In some embodiments of the present invention, the plasmid-based influenza virus reverse genetics system is provided, wherein the nucleic acid comprises a codon mutated to TAG, wherein the codon encoding a site of HA protein selected from one or more of the following:
C84, S86, S92, S126, E132, P135, G147, K170, K176, N179, S201, 1256, S53, K57, K62, 164, A65, L67, K71 or P82.

In some embodiments of the present invention, the plasmid-based influenza virus reverse genetics system is provided, wherein the PB1 gene, PA gene and/or NP gene in (1) contain one or more (e.g., 2, 3, 4 or 5) TAG codons.

In some embodiments of the present invention, the plasmid-based influenza virus reverse genetics system is provided, wherein the nucleic acid codon encoding the following site is mutated to TAG codon:
R52 site of PB1 protein,
R266 site of PA protein,
and/or
D101 site of NP protein.

In some embodiments of the present invention, the plasmid-based influenza virus reverse genetics system is provided, wherein the nucleic acid codons encoding the following sites are mutated to TAG codons:
R52 site of PB1 protein, R266 site of PA protein, S53 site of HA protein and D101 site of NP protein;
K33 site of PB2 protein, R266 site of PA protein, S53 site of HA protein and D101 site of NP protein;
or
K33 site of PB2 protein, R52 site of PB1 protein, S53 site of HA protein and D101 site of NP protein.

In some embodiments of the present invention, the plasmid-based influenza virus reverse genetics system is provided, wherein before mutation the influenza virus is a wild-type influenza virus; preferably, wild-type influenza virus A/WSN/1933.

In some embodiments of the present invention, the plasmid-based influenza virus reverse genetics system is provided, wherein:
the nucleic acid sequence of the PB2 gene unmutated is shown in SEQ ID NO: 1;
the nucleic acid sequence of the PB1 gene unmutated is shown in SEQ ID NO: 2;
the nucleic acid sequence of the PA gene unmutated is shown in SEQ ID NO: 3;
the nucleic acid sequence of the HA gene unmutated is shown in SEQ ID NO: 4;
the nucleic acid sequence of the NP gene unmutated is shown in SEQ ID NO: 5;
the nucleic acid sequence of the NA gene unmutated is shown in SEQ ID NO: 6;
the nucleic acid sequence of the M gene unmutated is shown in SEQ ID NO: 7;
and/or
the nucleic acid sequence of the NS gene unmutated is shown in SEQ ID NO: 8.

In some embodiments of the present invention, the plasmid-based influenza virus reverse genetics system is provided, wherein one or more of the TAG codons are located upstream of stop codon.

In some embodiments of the present invention, the plasmid-based influenza virus reverse genetics system is provided, wherein the amino acids at the positions encoded by one or more of the TAG codons are the same or different non-natural amino acids such as NAEK.

Further another aspect of the present invention relates to an influenza virus, which is obtained by rescuing through the plasmid-based influenza virus reverse genetics system described in any one of the items of the present invention.

In the present invention, the term “armed influenza virus” refers to an influenza virus with a special property unexisting in a wild-type influenza virus by modify influenza virus envelope protein via chemical means or by modifying influenza virus genome via genetic engineering means.

In the present invention, the term “bio-orthogonal reaction” refers to a chemical reaction that can be carried out in living cells or tissues without interfering with the biochemical reaction of the organism itself.

In the present invention, the term “click chemistry reaction” refers to a rapid and reliable chemical synthesis reaction to form various molecules by splicing small units.

In the present invention, the term “nanobody” contains only one heavy chain variable region (VHH), which is the smallest unit known to bind to target antigen.

In the present invention, the term “bio-orthogonal aminoacyl-tRNA synthetase” is derived from Archaea and has been artificially evolved. It cannot recognize natural amino acids, but can recognize non-natural amino acids such as NAEK.

The structural formula of NAEK is shown in Formula V below.

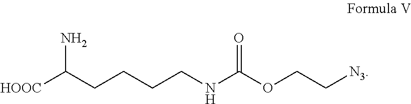 [SMILES: [N-]=[N+]=NCCOC(=O)NCCCCC(N)C(=O)O]

The present invention overcomes the disadvantages of low autoimmunogenicity of tumor antigen peptide vaccines, the disadvantages of systemic side effects of immune checkpoint inhibitors, and the problem of pathogenicity of influenza virus as an oncolytic virus.

The present invention is applied to tumor treatment, especially the treatment of lung tumors and lung metastatic tumors, and can be used in combination with other tumor immunotherapies.

### Beneficial Effects of the Present Invention

The present invention has achieved one or more of the following technical effects:
1) The replication-deficient influenza virus is a new influenza virus vaccine independently developed by the present team, which has good safety.
2) The influenza virus itself is a single-stranded negative-strand RNA virus and a natural mRNA vaccine carrier. In the present invention, the influenza virus is used to deliver a nanobody that can encode anti-pdl1.
3) The present invention utilizes a non-natural amino acid system to couple a tumor antigen peptide on the surface of influenza virus through bio-orthogonal reaction, and delivers the tumor antigen to the lung through the respiratory tract, especially for the treatment of lung tumors and lung metastatic tumors, with natural targeting advantages.
4) In the present disclosure, by utilizing the immunogenicity of influenza virus itself, the tumor microenvironment is changed, and it can be used in conjunction with a variety of tumor immunotherapies to enhance the effect.

### BRIEF DESCRIPTION OF THE DRAWINGS

FIG. 1 shows the expression efficiency of exogenous genes inserted into different viral gene truncations (PB1, PB2, PA) verified by using Glcui as an example.

FIG. 2A shows the verification by fluorescent western blot that exogenous peptides can be covalently coupled to the hemagglutinin protein of replication-deficient influenza virus.

FIG. 2B shows the verification by laser confocal microscopy that CpG can be anchored to influenza virus.

FIG. 3 shows the verification by laser confocal microscopy that P1-OVA1-FITC can promote the endocytosis and presentation of antigen peptides by dendritic cells.

FIG. 4A shows the efficiency of antigen presentation after dendritic cell processing in different dosing groups.

FIG. 4B shows the degree of dendritic cell activation in different dosing groups that was verified by the expression of dendritic cell surface markers.

FIG. 4C shows the degree of dendritic cell activation in different dosing groups that was verified by the secretion of cytokines by dendritic cells.

FIG. 5A shows the immunofluorescence photos of mouse lungs after nasal administration.

FIG. 5B shows the statistics of fluorescence intensity of antigen peptides in mouse lungs after nasal administration.

FIG. 5C shows the statistics of fluorescence intensity of CpG adjuvant in mouse lungs after nasal administration.

FIG. 6A shows the analysis of dendritic cells carrying antigens in mouse lungs and lymph nodes after nasal administration.

FIG. 6B shows the analysis of changes in immune cells in mouse lungs after nasal administration.

FIG. 6C shows the analysis of antigen-specific T lymphocytes in mouse lungs and spleens after nasal administration.

FIG. 7A shows the analysis of antibody levels in mouse serum after nasal administration.

FIG. 7B shows the analysis of antibody levels in mouse lung lavage fluid after nasal administration.

FIG. 8A shows the photos of pre-immunized mice against lung metastasis from melanoma.

FIG. 8B shows the statistical diagram of the effect of pre-immunized mice against lung metastasis from melanoma.

FIG. 8C shows the statistics of the content of pulmonary antigen-specific T cells in the pre-immunized mice against model of lung metastasis from melanoma.

FIG. 9A shows the administration flow chart of the vaccine therapeutic experiment.

FIG. 9B shows the statistics of the proportion of different immune cells in tumor tissues.

FIG. 9C shows the fluorescence slices of infiltrating T cells in tumor tissues.

FIG. 9D shows the statistics of the proportion of memory T cells and tissue-resident T cells after administration.

FIG. 10A shows the verification by gel electrophoresis that 5dxw or 5e03 fragments can be packaged into influenza virus.

FIG. 10B shows the verification by Western blot that P15dxw-OVA1 or P15e03-OVA1 can secrete 5dxw or 5e03 after infecting cells.

FIG. 10C shows the verification by fluorescence slices that P15dxw-OVA1 or P15e03-OVA1 can infect lungs to secrete antibodies after nasal administration.

FIG. 11A shows the imaging of small animals treated with nasal administration for lung metastasis of melanoma.

FIG. 11B shows the statistics of fluorescence intensity of lung tumors.

FIG. 11C shows the survival rate curve of mice after treatment.

FIG. 11D shows the immunohistochemical analysis of T cell infiltration in the lungs of tumor-bearing mice.

FIG. 11E shows the analysis of immune cells in the lungs, peripheral blood, and spleens of tumor-bearing mice.

FIG. 12A shows the verification of the therapeutic effect of P15dxw-4T1 in the mouse 4T1 model of lung metastasis from breast cancer.

FIG. 12B shows the verification of the therapeutic effect of P15dxw-CT26 in the mouse CT26 model of lung metastasis from colon cancer.

FIG. 13A shows the experimental flow chart for verifying the therapeutic effect of P15dxw-B16 on distal tumors.

FIG. 13B shows the imaging of animals with distal tumors treated with P15dxw-B16.

FIG. 13C shows the statistics of tumor size of distal tumors treated with P15dxw-B16.

FIG. 13D shows the photos of distal tumors treated with P15dxw-B16.

FIG. 13E shows the survival curve of mice bearing distal tumors treated with P15dxw-B16.

FIG. 13F shows the statistics of intratumoral antigen-specific T cells in the experiment of treating distal tumors with P15dxw-B16.

FIG. 14A shows the verification by taking P15dxw-OVA1 as an example that the vaccine can only replicate in transgenic cells and in the presence of unnatural amino acids.

FIG. 14B shows the verification by monitoring the weight of immunized mice that P15dxw-OVA1 or P1-OVA1 is absolutely safe as compared with wild-type viruses.

FIG. 14C shows that the safety of P15dxw-OVA1 or P1-OVA1 is proved by the pulmonary edema of mice.

FIG. 14D shows the H&E staining results of various tissues in different dosing groups.

The partial sequences involved in the present invention are as follows:

PB2
(SEQ ID NO: 1)
agcgaaagcaggtcaattatattcaatatggaaagaataaaagaactaaggaatctaatgtcgcagtctcgcactcgcgagatactca
caaaaaccaccgtggaccatatggccataatcaagaagtacacatcaggaagacaggagaagaacccagcacttaggatgaaatggat
gatggcaatgaaatatccaattacagcagacaagaggataacggaaatgattcctgagagaaatgagcagggacaaactttatggagt
aaaatgaatgacgccggatcagaccgagtgatggtatcacctctggctgtgacatggtggaataggaatggaccagtgacaagtacag
ttcattatccaaaaatctacaaaacttattttgaaaaagtcgaaaggttaaaacatggaacctttggccctgtccattttagaaacca
agtcaaaatacgtcgaagagttgacataaatcctggtcatgcagatctcagtgccaaagaggcacaggatgtaatcatggaagttgtt
ttccctaacgaagtgggagccaggatactaacatcggaatcgcaactaacgacaaccaaagagaagaaagaagaactccagggttgca
aaatttctcctctgatggtggcatacatgttggagagagaactggtccgcaaaacgagattcctcccagtggctggtggaacaagcag
tgtgtacattgaagtgttgcatttgacccaaggaacatgctgggaacagatgtacactccaggaggggaggcgaggaatgatgatgtt
gatcaaagcttaattattgctgctagaaacatagtaagaagagccacagtatcagcagatccactagcatctttattggagatgtgcc
acagcacgcagattggtggaataaggatggtaaacatccttaggcagaacccaacagaagagcaagccgtggatatttgcaaggctgc
aatgggactgagaattagctcatccttcagttttggtggattcacatttaagagaacaagcggatcatcagtcaagagagaggaagag
gtgcttacgggcaatcttcagacattgaagataagagtgcatgagggatatgaagagttcacaatggttgggagaagagcaacagcta
tactcagaaaagcaaccaggagattgattcagctgatagtgagtgggagagacgaacagtcgattgccgaagcaataattgtggccat
ggtattttcacaagaggattgtatgataaaagcagttagaggtgacctgaatttcgtcaatagggcgaatcagcgattgaatcccatg
caccaacttttgagacattttcagaaggatgcaaaggtgctctttcaaaattggggaattgaatccatcgacaatgtgatgggaatga
tcgggatattgcccgacatgactccaagcaccgagatgtcaatgagaggagtgagaatcagcaaaatgggggtagatgagtattccag
cgcggagaagatagtggtgagcattgaccgttttttgagagttagggaccaacgtgggaatgtactactgtctcccgaggagatcagt
gaaacacagggaacagagaaactgacaataacttactcatcgtcaatgatgtgggagattaatggtcctgaatcagtgttggtcaata
cctatcagtggatcatcagaaactgggaaactgttaaaattcagtggtcccagaatcctacaatgctgtacaataaaatggaatttga
gccatttcagtctttagttccaaaggccgttagaggccaatacagtgggtttgtgagaactctgttccaacaaatgagggatgtgctt
gggacatttgataccgctcagataataaaacttcttcccttcgcagccgctccaccaaagcaaagtagaacgcagttctcctcattga
ctataaatgtgaggggatcaggaatgagaatacttgtaaggggcaattctccagtattcaactacaacaagaccactaaaagactcac
agttctcggaaaggatgctggccctttaactgaagacccagatgaaggcacagctggagttgagtccgcagttctgagaggattcctc
attctgggcaaagaagacaggagatatggaccagcattaagcataaatgaactgagcaaccttgcgaaaggagagaaggctaatgtgc
taattgggcaaggagacgtggtgttggtaatgaaacggaaacggaactctagcatacttactgacagccagacagcgaccaaaagaat
tcggatggccatcaattagtgtcgaatagtttaaaaacgaccttgtttctact
PB1
(SEQ ID NO: 2)
agcgaaagcaggcaaaccatttgaatggatgtcaatccgactttacttttcttaaaagtgccagcacaaaatgctataagcacaactt
tcccttatactggagaccctccttacagccatgggacaggaacaggatacaccatggatactgtcaacaggacacatcagtactcaga
aaggggaagatggacaacaaacaccgaaactggagcaccgcaactcaacccgattgatgggccactgccagaagacaatgaaccaagt
ggttatgcccaaacagattgtgtattggaagcaatggccttccttgaggaatcccatcctggtatctttgagacctcgtgtcttgaaa
cgatggaggttgttcagcaaacacgagtggacaagctgacacaaggccgacagacctatgactggactctaaataggaaccagcctgc
tgcaacagcattggccaacacaatagaagtgttcagatcaaatggcctcacggccaatgaatctggaaggctcatagacttccttaag
gatgtaatggagtcaatgaacaaagaagaaatggagatcacaactcattttcagagaaagagacgagtgagagacaatatgactaaga
aaatggtgacacagagaacaataggtaaaaggaagcagagattgaacaaaaggagttatctaattagggcattaaccctgaacacaat
gaccaaagatgctgagagagggaagctaaaacggagagcaattgcaaccccagggatgcaaataagggggtttgtatactttgttgag
acactagcaaggagtatatgtgagaaacttgaacaatcaggattgccagttggaggcaatgagaagaaagcaaagttggcaaatgttg
taaggaagatgatgaccaattctcaggacactgaaatttctttcaccatcactggagataacaccaaatggaacgaaaatcagaaccc
tcggatgtttttggccatgatcacatatataaccagaaatcagcccgaatggttcagaaatgttctaagtattgctccaataatgttc
tcaaacaaaatggcgagactgggaaaggggtacatgtttgagagcaagagtatgaaaattagaactcaaatacctgcagaaatgctag
caagcatcgatttgaaatacttcaatgattcaactagaaagaagattgaaaaaatccggccgctcttaatagatgggactgcatcatt
gagccctggaatgatgatgggcatgttcaatatgttaagtactgtattaggcgtctccatcctgaatcttggacaaaagagacacacc
aagactacttactggtgggatggtcttcaatcttctgatgattttgctctgattgtgaatgcacccaatcatgaagggattcaagccg
gagtcaacaggttttatcgaacctgtaagctacttggaattaatatgagcaagaaaaagtcttacataaacagaacaggtacatttga
attcacaagttttttctatcgttatgggtttgttgccaatttcagcatggagcttcccagctttggggtgtctgggatcaacgagtct
gcggacatgagtattggagttactgtcatcaaaaacaatatgataaacaatgatcttggtccagcaaccgctcaaatggcccttcagc
tgttcatcaaagattacaggtacacgtaccggtgccatagaggtgacacacaaatacaaacccgaagatcatttgaaataaagaaact
gtgggagcaaacccattccaaagctggactgctggtctccgacggaggcccaaatttatacaacattagaaatctccacattcctgaa
gtctgcttgaaatgggaattaatggatgaggattaccaggggcgtttatgcaacccactgaacccatttgtcaaccataaagacattg
aatcagtgaacaatgcagtgataatgccagcacatggtccagccaaaaacatggagtatgatgctgttgcaacaacacactcctggat
ccccaaaagaaatcgatccatcttgaatacaagccaaagaggaatacttgaagatgaacaaatgtaccaaaagtgctgcaacttattt
gaaaaattcttccccagcagttcatacagaagaccagtcgggatatccagtatggtggaggctatggtttccagagcccgaattgatg
cacgaattgatttcgaatctggaaggataaagaaagaggagttcactgagatcatgaagatctgttccaccattgaagagctcagacg
gcaaaaatagtgaatttagcttgtccttcatgaaaaaatgccttgtttctact
PA
(SEQ ID NO: 3)
agcgaaagcaggtactgattcaaaatggaagattttgtgcgacaatgcttcaatccgatgattgtcgagcttgcggaaaaggcaatga
aagagtatggagaggacctgaaaatcgaaacaaacaaatttgcagcaatatgcactcacttggaagtgtgcttcatgtattcagattt
tcacttcatcgatgagcaaggcgagtcaatagtcgtagaacttggcgatccaaatgcacttttgaagcacagatttgaaataatcgag
ggaagagatcgcacaatagcctggacagtaataaacagtatttgcaacactacaggggctgagaaaccaaagtttctaccagatttgt
atgattacaagaagaatagattcatcgaaattggagtaacaaggagagaagttcacatatactatctggaaaaggccaataaaattaa
atctgagaagacacacatccacattttctcattcactggggaggaaatggccacaaaggccgactacactctcgatgaagaaagcagg
gctaggatcaaaaccaggctattcaccataagacaagaaatggctagcagaggcctctgggattcctttcgtcagtccgagagaggcg
aagagacaattgaagaaagatttgaaatcacaggaacaatgcgcaagcttgccgaccaaagtctcccgccaaacttctccagccttga
aaaatttagagcctatgtggatggattcgaaccgaacggctacattgagggcaagctttctcaaatgtccaaagaagtaaatgctaga
attgaaccttttttgaaatcaacaccacgaccacttagacttccggatgggcctccctgttctcageggtccaaattcctgctgatgg
atgccttaaaattaagcattgaggacccaagtcatgagggagaggggataccgctatatgatgcaatcaaatgcatgagaacattctt
tggatggaaggaacccaatgttgttaaaccacacgaaaagggaataaatccaaattatcttctgtcatggaagcaagtactggcagaa
ctgcaggacattgagaatgaggagaaaattccaaggactaaaaatatgaagaaaacgagtcagttaaagtgggcacttggtgagaaca
tggcaccagaaaaggtagactttgacgattgtaaagatgtaggcgatttgaagcaatatgatagtgatgaaccagaattgaggtcgct
tgcaagttggattcagaatgagttcaacaaggcatgtgaactgaccgattcaagctggatagagctcgatgagattggagaagatgcg
gctccaattgaacacattgcaagcatgagaaggaattatttcacagcagaggtgtctcattgcagagccacagaatacataatgaagg
gggtgtacatcaatactgccttgcttaatgcatcctgtgcagcaatggatgatttccaattaattccaatgataagcaagtgtagaac
taaggagggaaggcgaaagaccaatttgtacggtttcatcataaaaggaagatcccacttaaggaatgacaccgatgtggtaaacttt
gtgagcatggagttttccctcactgacccaagacttgaaccacacaaatgggagaagtactgtgttcttgaggtaggagatatgcttc
taagaagtgccataggccatgtgtcaaggcctatgttcttgtatgtgaggacaaatggaacctcaaaaattaaaatgaaatgggggat
ggaaatgaggcgttgcctccttcagtcacttcaacaaatcgagagtatgattgaagctgagtcctctgtcaaggagaaagacatgacc
aaagagttctttgaaaacaaatcagaaacatggcccgttggagagtcccccaaaggagtggaggaaggttccattgggaaggtctgca
gaactttattggcaaagtcggtattcaacagcttgtatgcatctccacaactagaaggattttcagctgaatcaagaaaactgcttct
tatcgttcaggctcttagggacaacctggaacctgggacctttgatcttggggggctatatgaagcaattgaggagtgcctgattaat
gatccctgggttttgcttaatgcttcttggttcaactccttcctcacacatgcattgagatagttgtggcaatgctactatttgctat
ccatactgtccaaaaaagtaccttgtttctact
HA
(SEQ ID NO: 4)
agcaaaagcaggggaaaataaaaacaaccaaaatgaaggcaaaactactggtcctgttatatgcatttgtagctacagatgcagacac
aatatgtataggctaccatgcgaacaactcaaccgacactgttgacacaatactcgagaagaatgtggcagtgacacattctgttaac
ctgctcgaagacagccacaacgggaaactatgtaaattaaaaggaatagccccactacaattggggaaatgtaacatcaccggatggc
tcttgggaaatccagaatgcgactcactgcttccagcgagatcatggtcctacattgtagaaacaccaaactctgagaatggagcatg
ttatccaggagatctcatcgactatgaggaactgagggagcaattgagctcagtatcatcattagaaagattcgaaatatttcccaag
gaaagttcatggcccaaccacacattcaacggagtaacagtatcatgctcccataggggaaaaagcagtttttacagaaatttgctat
ggctgacgaagaagggggattcatacccaaagctgaccaattcctatgtgaacaataaagggaaagaagtccttgtactatggggtgt
tcatcacccgtctagcagtgatgagcaacagagtctctatagtaatggaaatgcttatgtctctgtagcgtcttcaaattataacagg
agattcaccccggaaatagctgcaaggcccaaagtaagagatcaacatgggaggatgaactattactggaccttgctagaacccggag
acacaataatatttgaggcaactggtaatctaatagcaccatggtatgctttcgcactgagtagagggtttgagtccggcatcatcac
ctcaaacgcgtcaatgcatgagtgtaacacgaagtgtcaaacaccccagggagctataaacagcaatctccctttccagaatatacac
ccagtcacaataggagagtgcccaaaatatgtcaggagtaccaaattgaggatggttacaggactaagaaacatcccatccattcaat
acagaggtctatttggagccattgctggttttattgaggggggatggactggaatgatagatggatggtatggttatcatcatcagaa
tgaacagggatcaggctatgcagcggatcaaaaaagcacacaaaatgccattaacgggattacaaacaaggtgaactctgttatcgag
aaaatgaacactcaattcacagctgtgggtaaagaattcaacaacttagaaaaaaggatggaaaatttaaataaaaaagttgatgatg
ggtttctggacatttggacatataatgcagaattgttagttctactggaaaatgaaaggactttggatttccatgacttaaatgtgaa
gaatctgtacgagaaagtaaaaagccaattaaagaataatgccaaagaaatcggaaatgggtgttttgagttctaccacaagtgtgac
aatgaatgcatggaaagtgtaagaaatgggacttatgattatccaaaatattcagaagaatcaaagttgaacagggaaaagatagatg
gagtgaaattggaatcaatgggggtgtatcagattctggcgatctactcaactgtcgccagttcactggtgcttttggtctccctggg
ggcaatcagtttctggatgtgttctaatgggtctttgcagtgcagaatatgcatctgagattaggatttcagaaatataaggaaaaac
acccttgtttctact
NP
(SEQ ID NO: 5)
agcaaaagcagggtagataatcactcacagagtgacatcgaaatcatggcgaccaaaggcaccaaacgatcttacgaacagatggaga
ctgatggagaacgccagaatgccactgaaatcagagcatctgtcggaaaaatgattgatggaattggacgattctacatccaaatgtg
caccgaacttaaactcagtgattatgagggacggctgattcagaacagcttaacaatagagagaatggtgctctctgcttttgacgag
aggaggaataaatatctagaagaacatcccagtgcggggaaagatcctaagaaaactggaggacctatatacaggagagtagatggaa
agtggaggagagaactcatcctttatgacaaagaagaaataagacgaatctggcgccaagctaataatggtgacgatgcaacggctgg
tctgactcacatgatgatctggcactccaatttgaatgatgcaacttaccagaggacaagagctcttgttcgcacaggaatggatccc
aggatgtgctcactgatgcagggttcaaccctccctaggaggtctggggccgcaggtgctgcagtcaaaggagttggaacaatggtga
tggaattgatcagaatgatcaaacgtgggatcaatgatcggaacttctggaggggtgagaatggacggagaacaaggattgcttatga
aagaatgtgcaacattctcaaagggaaatttcaaacagctgcacaaagaacaatggtggatcaagtgagagagagccggaatccagga
aatgctgagttcgaagatctcatctttttagcacggtctgcactcatattgagagggtcagttgctcacaagtcctgcctgcctgcct
gtgtgtatggatctgccgtagccagtggatacgactttgaaagagagggatactctctagtcggaatagaccctttcagactgcttca
aaacagccaagtatacagcctaatcagaccaaatgagaatccagcacacaagagtcaactggtgtggatggcatgccattctgctgca
tttgaagatctaagagtatcaagcttcatcagagggacgaaagtggtcccaagagggaagctttccactagaggagttcaaattgctt
ccaatgaaaacatggagactatggaatcaagtacccttgaactgagaagcagatactgggccataaggaccagaagtggagggaacac
caatcaacagagggcttcctcgggccaaatcagcatacaacctacgttctcagtacagagaaatctcccttttgacagaccaaccatt
atggcagcattcactgggaatacagaggggagaacatctgacatgagaaccgaaatcataaggctgatggaaagtgcaagaccagaag
atgtgtctttccaggggggggagtcttcgagctctcggacgaaaaggcaacgagcccgatcgtgccctcctttgacatgagtaatgaa
ggatcttatttcttcggagacaatgcagaggagtacgacaattaaagaaaaatacccttgtttctact
NA
(SEQ ID NO: 6)
agcgaaagcaggagtttaaatgaatccaaaccagaaaataataaccattgggtcaatctgtatggtagtcggaataattagcctaata
ttgcaaataggaaatataatctcaatatggattagccattcaattcaaaccggaaatcaaaaccatactggaatatgcaaccaaggca
gcattacctataaagttgttgctgggcaggactcaacttcagtgatattaaccggcaattcatctctttgtcccatccgtggggggct
atacacagcaaagacaatggcataagaattggttccaaaggagacgtttttgtcataagagagccttttatttcatgttctcacttgg
aatgcaggaccttttttctgactcaaggcgccttactgaatgacaagcattcaagggggacctttaaggacagaagcccttatagggc
cttaatgagctgccctgtcggtgaagctccgtccccgtacaattcaaggtttgaatcggttgcttggtcagcaagtgcatgtcatgat
ggaatgggctggctaacaatcggaatttctggtccagatgatggagcagtggctgtattaaaatacaaccgcataataactgaaacca
taaaaagttggaggaagaatatattgagaacacaagagtctgaatgtacctgtgtaaatggttcatgttttaccataatgaccgatgg
cccaagtgatgggctggcctcgtacaaaattttcaagatcgagaaggggaaggttactaaatcgatagagttgaatgcacctaattct
cactacgaggaatgttcctgttaccctgataccggcaaagtgatgtgtgtgtgcagagacaattggcacggttcgaaccgaccatggg
tgtccttcgaccaaaacctagattataaaataggatacatctgcagtggggttttcggtgacaacccgcgtcccaaagatggaacagg
cagctgtggcccagtgtctgctgatggagcaaacggagtaaagggattttcatataagtatggcaatggtgtttggataggaaggact
aaaagtgacagttccagacatgggtttgagatgatttgggatcctaatggatggacagagactgatagtaggttctctatgagacaag
atgttgtggcaataactaatcggtcagggtacagcggaagtttcgttcaacatcctgagctaacagggctagactgtatgaggccttg
cttctgggttgaattaatcagggggctacctgaggaggacgcaatctggactagtgggagcatcatttctttttgtggtgtgaatagt
gatactgtagattggtcttggccagacggtgctgagttgccgttcaccattgacaagtagtttgttcaaaaaactccttgtttctact
M
(SEQ ID NO: 7)
agcaaaagcaggtagatattgaaagatgagtcttctaaccgaggtcgaaacgtacgttctctctatcgtcccgtcaggccccctcaaa
gccgagatcgcacagagacttgaagatgtctttgcagggaagaacaccgatcttgaggttctcatggaatggctaaagacaagaccaa
tcctgtcacctctgactaaggggattttaggatttgtgttcacgctcaccgtgcccagtgagcggggactgcagcgtagacgctttgt
ccaaaatgctcttaatgggaacggagatccaaataacatggacaaagcagttaaactgtataggaagcttaagagggagataacattc
catggggccaaagaaatagcactcagttattctgctggtgcacttgccagttgtatgggcctcatatacaacaggatgggggctgtga
ccactgaagtggcatttggcctggtatgcgcaacctgtgaacagattgctgactcccagcatcggtctcataggcaaatggtgacaac
aaccaatccactaatcagacatgagaacagaatggttctagccagcactacagctaaggctatggagcaaatggctggatcgagtgag
caagcagcagaggccatggatattgctagtcaggccaggcaaatggtgcaggcgatgagaaccgttgggactcatcctagctccagtg
ctggtctaaaagatgatcttcttgaaaatttgcaggcctatcagaaacgaatgggggtgcagatgcaacgattcaagtgatcctctcg
tcattgcagcaaatatcattggaatcttgcacttgatattgtggattcttgatcgtctttttttcaaatgcatttatcgtcgctttaa
atacggtttgaaaagagggccttctacggaaggagtgccagagtctatgagggaagaatatcgaaaggaacagcagaatgctgtggat
gttgacgatggtcattttgtcaacatagagctggagtaaaaaactaccttgtttctact
NS
(SEQ ID NO: 8)
agcaaaagcagggtgacaaagacataatggatccaaacactgtgtcaagctttcaggtagattgctttctttggcatgtccgcaaaag
agttgcagaccaagaactaggtgatgccccattccttgateggcttcgccgagatcagaagtccctaagaggaagaggcagcactctt
ggtctggacatcgaaacagccacccgtgctggaaagcaaatagtggagcggattctgaaggaagaatctgatgaggcactcaaaatga
ccatggcctctgtacctgcatcgcgctacctaactgacatgactcttgaggaaatgtcaaggcactggttcatgctcatgcccaagca
gaaagtggcaggccctctttgtatcagaatggaccaggcgatcatggataagaacatcatactgaaagcgaacttcagtgtgattttt
gaccggctggagactctaatattactaagggccttcaccgaagaggggacaattgttggcgaaatttcaccactgccctctcttccag
gacatactgatgaggatgtcaaaaatgcagttggggtcctcatcggaggacttgaatggaataataacacagttcgagtctctgaaac
tctacagagattcgcttggagaagcagtaatgagaatgggagacctccactcactccaaaacagaaacggaaaatggcgggaacaatt
aggtcagaagtttgaagaaataagatggttgattgaagaagtgagacacagactgaagataacagagaatagttttgagcaaataaca
tttatgcaagccttacaactattgcttgaagtggagcaagagataagaactttctcgtttcagcttatttaataataaaaaacaccct
tgtttctact
PB2-Gluci
(SEQ ID NO: 11)
agcgaaagcaggtcaattatattcaatatggaaagaataaaagaactaaggaatctaatgtcgcagtctcgcactcgcgagatactca
caaaaaccaccgtggaccatatggccataatcaagaagtacacatcaggaagacaggagaagaacccagcacttaggatgaaatggat
gatggcaatgaaatatccaattacagcagacaagaggataacggaaatgattcctgagagaaatgagcagggacaaactttatggagt
aaaatgaatgacgccggatcagaccgagtgatggtatcacctctggctgtgacatggtggaataggaatggaccagtgacaagtacag
ttcattatccaaaaatctacaaaacttattttgaaaaagtcgaaaggttaaaacatggaacctttggccctgtccattttagaaacca
agtcaaaatacgtcgaagagttgacataaatcctggtcatgcagatctcagtgccaaagaggcacaggatgtaatcatggaagttgtt
ttccctaacgaagtgggagccaggatactaacatcggaatcgcaactaacgacaaccaaagagaagaaagaagaactccagggttgca
aaatttctcctctgatggtggcatacatgttggagagagaactggtccgcaaaacgagattcctcccagtggctggtggaacaagcag
tgtgtacattgaagtgttgcatttgacccaaggaacatgctgggaacagatgtacactccaggaggggaggcgaggaatgatgatgtt
gatcaaagcttaattattgctgctagaaacatagtaagaagagccacagtatcagcagatccactagcatctttattggagatgtgcc
acagcacgcagattggtggaataaggatggtaaacatccttaggcagaacccaacagaagagcaagccgtggatatttgcaaggctgc
aatgggactgagaattagctcatccttcagttttggtggattcacatttaagagaacaagcggatcatcagtcaagagagaggaagag
gtgcttacgggcaatcttcagacattgaagataagagtgcatgagggatatgaagagttcacaatggttgggagaagagcaacagcta
tactcagaaaagcaaccaggagattgattcagctgatagtgagtgggagagacgaacagtcgattgccgaagcaataattgtggccat
ggtattttcacaagaggattgtatgataaaagcagttagaggtgacctgaatttcgtcaatagggcgaatcagcgattgaatcccatg
caccaacttttgagacattttcagaaggatgcaaaggtgctctttcaaaattggggaattgaatccatcgacaatgtgatgggaatga
tcgggatattgcccgacatgactccaagcaccgagatgtcaatgagaggagtgagaatcagcaaaatgggggtagatgagtattccag
cgcggagaagatagtggtgagcattgaccgttttttgagagttagggaccaacgtgggaatgtactactgtctcccgaggagatcagt
gaaacacagggaacagagaaactgacaataacttactcatcgtcaatgatgtgggagattaatggtcctgaatcagtgttggtcaata
cctatcagtggatcatcagaaactgggaaactgttaaaattcagtggtcccagaatcctacaatgctgtacaataaaatggaatttga
gccatttcagtctttagttccaaaggccgttagaggccaatacagtgggtttgtgagaactctgttccaacaaatgagggatgtgctt
gggacatttgataccgctcagataataaaacttcttcccttcgcagccgctccaccaaagcaaagtagaacgcagttctcctcattga
ctataaatgtgaggggatcaggaatgagaatacttgtaaggggcaattctccagtattcaactacaacaagaccactaaaagactcac
agttctcggaaaggatgctggccctttaactgaagacccagatgaaggcacagctggagttgagtccgcagttctgagaggattcctc
attctgggcaaagaagacaggagatatggaccagcattaagcataaatgaactgagcaaccttgcgaaaggagagaaggctaatgtgc
taattgggcaaggagacgtggtactagtgatgaagaggaagagaaatagctctatcttgacggattcacaaacggcaactaagaggat
ccgtatggctattaacggttctggcgccaccaacttctccctgctgaagcaggctggcgatgtggaggagaaccctgggcccatggga
gtcaaagttctgtttgccctgatctgcatcgctgtggccgaggccaagcccaccgagaacaacgaagacttcaacatcgtggccgtgg
ccagcaacttegcgaccacggatctcgatgctgaccgcgggaagttgcccggcaagaagctgccgctggaggtgctcaaagagttgga
agccaatgcccggaaagctggctgcaccaggggctgtctgatctgcctgtcccacatcaagtgcacgcccaagatgaagaagttcatc
ccaggacgctgccacacctacgaaggcgacaaagagtccgcacagggcggcataggcgaggcgatcgtcgacattcctgagattcctg
ggttcaaggacttggagcccttggagcagttcatcgcacaggtcgatctgtgtgtggactgcacaactggctgcctcaaagggcttgc
caacgtgcagtgttctgacctgctcaagaagtggctgccgcaacgctgtgcgacctttgccagcaagatccagggccaggtggacaag
atcaagggggccggtggtgactaagcgaaaggagagaaggctaatgtgctaattgggcaaggagacgtggtgttggtaatgaaacgga
aacggaactctagcatacttactgacagccagacagcgaccaaaagaattcggatggccatcaattagtgtcgaatagtttaaaaacg
accttgtttctact
PB1-Gluci
(SEQ ID NO: 12)
atggatgtcaatccgactttacttttcttaaaagtgccagcacaaaatgctataagcacaactttcccttatactggagaccctcctt
acagccatgggacaggaacaggatacaccatggatactgtcaacaggacacatcagtactcagaaaggggaagatggacaacaaacac
cgaaactggagcaccgcaactcaacccgattgatgggccactgccagaagacaatgaaccaagtggttatgcccaaacagattgtgta
ttggaagcaatggccttccttgaggaatcccatcctggtatctttgagacctcgtgtcttgaaacgatggaggttgttcagcaaacac
gagtggacaagctgacacaaggccgacagacctatgactggactctaaataggaaccagcctgctgcaacagcattggccaacacaat
agaagtgttcagatcaaatggcctcacggccaatgaatctggaaggctcatagacttccttaaggatgtaatggagtcaatgaacaaa
gaagaaatggagatcacaactcattttcagagaaagagacgagtgagagacaatatgactaagaaaatggtgacacagagaacaatag
gtaaaaggaagcagagattgaacaaaaggagttatctaattagggcattaaccctgaacacaatgaccaaagatgctgagagagggaa
gctaaaacggagagcaattgcaaccccagggatgcaaataagggggtttgtatactttgttgagacactagcaaggagtatatgtgag
aaacttgaacaatcaggattgccagttggaggcaatgagaagaaagcaaagttggcaaatgttgtaaggaagatgatgaccaattctc
aggacactgaaatttctttcaccatcactggagataacaccaaatggaacgaaaatcagaaccctcggatgtttttggccatgatcac
atatataaccagaaatcagcccgaatggttcagaaatgttctaagtattgctccaataatgttctcaaacaaaatggcgagactggga
aaggggtacatgtttgagagcaagagtatgaaaattagaactcaaatacctgcagaaatgctagcaagcatcgatttgaaatacttca
atgattcaactagaaagaagattgaaaaaatccggccgctcttaatagatgggactgcatcattgagccctggaatgatgatgggcat
gttcaatatgttaagtactgtattaggcgtctccatcctgaatcttggacaaaagagacacaccaagactacttactgggggatggtc
ttcaatcttctgatgattttgctctgattgtgaatgcacccaatcatgaagggattcaagccggagtcaacaggttttatcgaacctg
taagctacttggaattaatatgagcaagaaaaagtcttacataaacagaacaggtacatttgaattcacaagttttttctatcgttat
gggtttgttgccaatttcagcatggagcttcccagctttggggtgtctgggatcaacgagtctgcggacatgagtattggagttactg
tcatcaaaaacaatatgataaacaatgatcttggtccagcaaccgctcaaatggcccttcagctgttcatcaaagattacaggtacac
gtaccggtgccatagaggtgacacacaaatacaaacccgaagatcatttgaaataaagaaactgtgggagcaaacccattccaaagct
ggactgctggtctccgacggaggcccaaatttatacaacattagaaatctccacattcctgaagtctgcttgaaatgggaattaatgg
atgaggattaccaggggcgtttatgcaacccactgaacccatttgtcaaccataaagacattgaatcagtgaacaatgcagtgataat
gccagcacatggtccagccaaaaacatggagtatgatgctgttgcaacaacacactcctggatccccaaaagaaatcgatccatcttg
aatacaagccaaagaggaatacttgaagatgaacaaatgtaccaaaagtgctgcaacttatttgaaaaattcttccccagcagttcat
acagaagaccagtcgggatatccagtatggtggaggctatggtttccagagcccgaattgatgcacgaattgatttcgaatctggaag
gataaagaaagaggagttcactgagatcatgaaaatttgcagtacaatcgaggaacttcggagacagaagggttctggcgccaccaac
ttctccctgctgaagcaggctggcgatgtggaggagaaccctgggcccatgggagtcaaagttctgtttgccctgatctgcatcgctg
tggccgaggccaagcccaccgagaacaacgaagacttcaacatcgtggccgtggccagcaacttcgcgaccacggatctcgatgctga
ccgcgggaagttgcccggcaagaagctgccgctggaggtgctcaaagagttggaagccaatgcccggaaagctggctgcaccaggggc
tgtctgatctgcctgtcccacatcaagtgcacgcccaagatgaagaagttcatcccaggacgctgccacacctacgaaggcgacaaag
agtccgcacagggcggcataggcgaggcgatcgtcgacattcctgagattcctgggttcaaggacttggagcccttggagcagttcat
cgcacaggtcgatctgtgtgtggactgcacaactggctgcctcaaagggcttgccaacgtgcagtgttctgacctgctcaagaagtgg
ctgccgcaacgctgtgcgacctttgccagcaagatccagggccaggtggacaagatcaagggggccggtggtgactaa
PA-Gluci
(SEQ ID NO: 13)
atggaagattttgtgcgacaatgcttcaatccgatgattgtcgagcttgcggaaaaggcaatgaaagagtatggagaggacctgaaaa
tcgaaacaaacaaatttgcagcaatatgcactcacttggaagtgtgcttcatgtattcagattttcacttcatcgatgagcaaggcga
gtcaatagtcgtagaacttggcgatccaaatgcacttttgaagcacagatttgaaataatcgagggaagagatcgcacaatagcctgg
acagtaataaacagtatttgcaacactacaggggctgagaaaccaaagtttctaccagatttgtatgattacaagaagaatagattca
tcgaaattggagtaacaaggagagaagttcacatatactatctggaaaaggccaataaaattaaatctgagaagacacacatccacat
tttctcattcactggggaggaaatggccacaaaggccgactacactctcgatgaagaaagcagggctaggatcaaaaccaggctattc
accataagacaagaaatggctagcagaggcctctgggattcctttcgtcagtccgagagaggcgaagagacaattgaagaaagatttg
aaatcacaggaacaatgcgcaagcttgccgaccaaagtctcccgccaaacttctccagccttgaaaaatttagagcctatgtggatgg
attcgaaccgaacggctacattgagggcaagctttctcaaatgtccaaagaagtaaatgctagaattgaaccttttttgaaatcaaca
ccacgaccacttagacttccggatgggcctccctgttctcagcggtccaaattcctgctgatggatgccttaaaattaagcattgagg
acccaagtcatgagggagaggggataccgctatatgatgcaatcaaatgcatgagaacattctttggatggaaggaacccaatgttgt
taaaccacacgaaaagggaataaatccaaattatcttctgtcatggaagcaagtactggcagaactgcaggacattgagaatgaggag
aaaattccaaggactaaaaatatgaagaaaacgagtcagttaaagtgggcacttggtgagaacatggcaccagaaaaggtagactttg
acgattgtaaagatgtaggcgatttgaagcaatatgatagtgatgaaccagaattgaggtcgcttgcaagttggattcagaatgagtt
caacaaggcatgtgaactgaccgattcaagctggatagagctcgatgagattggagaagatgcggctccaattgaacacattgcaagc
atgagaaggaattatttcacagcagaggtgtctcattgcagagccacagaatacataatgaagggggtgtacatcaatactgccttgc
ttaatgcatcctgtgcagcaatggatgatttccaattaattccaatgataagcaagtgtagaactaaggagggaaggcgaaagaccaa
tttgtacggtttcatcataaaaggaagatcccacttaaggaatgacaccgatgtggtaaactttgtgagcatggagttttccctcact
gacccaagacttgaaccacacaaatgggagaagtactgtgttcttgaggtaggagatatgcttctaagaagtgccataggccatgtgt
caaggcctatgttcttgtatgtgaggacaaatggaacctcaaaaattaaaatgaaatgggggatggaaatgaggcgttgcctccttca
gtcacttcaacaaatcgagagtatgattgaagctgagtcctctgtcaaggagaaagacatgaccaaagagttctttgaaaacaaatca
gaaacatggcccgttggagagtcccccaaaggagtggaggaaggttccattgggaaggtctgcagaactttattggcaaagtcggtat
tcaacagcttgtatgcatctccacaactagaaggattttcagctgaatcaagaaaactgcttcttatcgttcaggctcttagggacaa
cctggaacctgggacctttgatcttggggggctatatgaagcaattgaggagtgcctgattaatgatccctgggttttgcttaacgcc
agctggtttaattcttttttgacgcacgcgctatcaggttctggcgccaccaacttctccctgctgaagcaggctggcgatgtggagg
agaaccctgggcccatgggagtcaaagttctgtttgccctgatctgcatcgctgtggccgaggccaagcccaccgagaacaacgaaga
cttcaacatcgtggccgtggccagcaacttcgcgaccacggatctcgatgctgaccgcgggaagttgcccggcaagaagctgccgctg
gaggtgctcaaagagttggaagccaatgcccggaaagctggctgcaccaggggctgtctgatctgcctgtcccacatcaagtgcacgc
ccaagatgaagaagttcatcccaggacgctgccacacctacgaaggcgacaaagagtccgcacagggcggcataggcgaggcgatcgt
cgacattcctgagattcctgggttcaaggacttggagcccttggagcagttcatcgcacaggtcgatctgtgtgtggactgcacaact
ggctgcctcaaagggcttgccaacgtgcagtgttctgacctgctcaagaagtggctgccgcaacgctgtgcgacctttgccagcaaga
tccagggccaggtggacaagatcaagggggccggtggtgactaa
PB2-5dxw
(SEQ ID NO: 14)
agcgaaagcaggtcaattatattcaatatggaaagaataaaagaactaaggaatctaatgtcgcagtctcgcactcgcgagatactca
caaaaaccaccgtggaccatatggccataatcaagaagtacacatcaggaagacaggagaagaacccagcacttaggatgaaatggat
gatggcaatgaaatatccaattacagcagacaagaggataacggaaatgattcctgagagaaatgagcagggacaaactttatggagt
aaaatgaatgacgccggatcagaccgagtgatggtatcacctctggctgtgacatggtggaataggaatggaccagtgacaagtacag
ttcattatccaaaaatctacaaaacttattttgaaaaagtcgaaaggttaaaacatggaacctttggccctgtccattttagaaacca
agtcaaaatacgtcgaagagttgacataaatcctggtcatgcagatctcagtgccaaagaggcacaggatgtaatcatggaagttgtt
ttccctaacgaagtgggagccaggatactaacatcggaatcgcaactaacgacaaccaaagagaagaaagaagaactccagggttgca
aaatttctcctctgatggtggcatacatgttggagagagaactggtccgcaaaacgagattcctcccagtggctggtggaacaagcag
tgtgtacattgaagtgttgcatttgacccaaggaacatgctgggaacagatgtacactccaggaggggaggcgaggaatgatgatgtt
gatcaaagcttaattattgctgctagaaacatagtaagaagagccacagtatcagcagatccactagcatctttattggagatgtgcc
acagcacgcagattggtggaataaggatggtaaacatccttaggcagaacccaacagaagagcaagccgtggatatttgcaaggctgc
aatgggactgagaattagctcatccttcagttttggtggattcacatttaagagaacaagcggatcatcagtcaagagagaggaagag
gtgcttacgggcaatcttcagacattgaagataagagtgcatgagggatatgaagagttcacaatggttgggagaagagcaacagcta
tactcagaaaagcaaccaggagattgattcagctgatagtgagtgggagagacgaacagtcgattgccgaagcaataattgtggccat
ggtattttcacaagaggattgtatgataaaagcagttagaggtgacctgaatttcgtcaatagggcgaatcagcgattgaatcccatg
caccaacttttgagacattttcagaaggatgcaaaggtgctctttcaaaattggggaattgaatccatcgacaatgtgatgggaatga
tcgggatattgcccgacatgactccaagcaccgagatgtcaatgagaggagtgagaatcagcaaaatgggggtagatgagtattccag
cgcggagaagatagtggtgagcattgaccgttttttgagagttagggaccaacgtgggaatgtactactgtctcccgaggagatcagt
gaaacacagggaacagagaaactgacaataacttactcatcgtcaatgatgtgggagattaatggtcctgaatcagtgttggtcaata
cctatcagtggatcatcagaaactgggaaactgttaaaattcagtggtcccagaatcctacaatgctgtacaataaaatggaatttga
gccatttcagtctttagttccaaaggccgttagaggccaatacagtgggtttgtgagaactctgttccaacaaatgagggatgtgctt
gggacatttgataccgctcagataataaaacttcttcccttcgcagccgctccaccaaagcaaagtagaacgcagttctcctcattga
ctataaatgtgaggggatcaggaatgagaatacttgtaaggggcaattctccagtattcaactacaacaagaccactaaaagactcac
agttctcggaaaggatgctggccctttaactgaagacccagatgaaggcacagctggagttgagtccgcagttctgagaggattcctc
attctgggcaaagaagacaggagatatggaccagcattaagcataaatgaactgagcaaccttgcgaaaggagagaaggctaatgtgc
taattgggcaaggagacgtggtactagtgatgaagaggaagagaaatagctctatcttgacggattcacaaacggcaactaagaggat
ccgtatggctattaacggttctggcgccaccaacttctccctgctgaagcaggctggcgatgtggaggagaaccctgggcccatggag
acagacaccctgctgctgtgggtgctgctgctttgggtgcctggatctacaggagatatggcccaagtgcaactggtggaaacaggcg
gcggcttagtgcaacctggaggcagccttagactgagctgtacagctagcggatttaccttcagcatgcacgccatgacctggtacag
acaggcccccggaaaacagagagagctggtggctgtgattaccagccacggagatagagccaactacaccgacagcgtgagaggcaga
ttcaccatcagcagagacaacaccaagaacatggtgtacctgcagatgaacagcctgaagcccgaggacaccgccgtgtattattgca
atgtgcccagatacgacagctggggccaaggcacacaagtgacagtgagcagcggaggactgcccgaaactggaggagaacaaaaact
gatcagcgaggaggacctgtaagcgaaaggagagaaggctaatgtgctaattgggcaaggagacgtggtgttggtaatgaaacggaaa
cggaactctagcatacttactgacagccagacagcgaccaaaagaattcggatggccatcaattagtgtcgaatagtttaaaaacgac
cttgtttctact
PB1-5dxw
(SEQ ID NO: 15)
atggatgtcaatccgactttacttttcttaaaagtgccagcacaaaatgctataagcacaactttcccttatactggagaccctcctt
acagccatgggacaggaacaggatacaccatggatactgtcaacaggacacatcagtactcagaaaggggaagatggacaacaaacac
cgaaactggagcaccgcaactcaacccgattgatgggccactgccagaagacaatgaaccaagtggttatgcccaaacagattgtgta
ttggaagcaatggccttccttgaggaatcccatcctggtatctttgagacctcgtgtcttgaaacgatggaggttgttcagcaaacac
gagtggacaagctgacacaaggccgacagacctatgactggactctaaataggaaccagcctgctgcaacagcattggccaacacaat
agaagtgttcagatcaaatggcctcacggccaatgaatctggaaggctcatagacttccttaaggatgtaatggagtcaatgaacaaa
gaagaaatggagatcacaactcattttcagagaaagagacgagtgagagacaatatgactaagaaaatggtgacacagagaacaatag
gtaaaaggaagcagagattgaacaaaaggagttatctaattagggcattaaccctgaacacaatgaccaaagatgctgagagagggaa
gctaaaacggagagcaattgcaaccccagggatgcaaataagggggtttgtatactttgttgagacactagcaaggagtatatgtgag
aaacttgaacaatcaggattgccagttggaggcaatgagaagaaagcaaagttggcaaatgttgtaaggaagatgatgaccaattctc
aggacactgaaatttctttcaccatcactggagataacaccaaatggaacgaaaatcagaaccctcggatgtttttggccatgatcac
atatataaccagaaatcagcccgaatggttcagaaatgttctaagtattgctccaataatgttctcaaacaaaatggcgagactggga
aaggggtacatgtttgagagcaagagtatgaaaattagaactcaaatacctgcagaaatgctagcaagcatcgatttgaaatacttca
atgattcaactagaaagaagattgaaaaaatccggccgctcttaatagatgggactgcatcattgagccctggaatgatgatgggcat
gttcaatatgttaagtactgtattaggcgtctccatcctgaatcttggacaaaagagacacaccaagactacttactggtgggatggt
cttcaatcttctgatgattttgctctgattgtgaatgcacccaatcatgaagggattcaagccggagtcaacaggttttatcgaacct
gtaagctacttggaattaatatgagcaagaaaaagtcttacataaacagaacaggtacatttgaattcacaagttttttctatcgtta
tgggtttgttgccaatttcagcatggagcttcccagctttggggtgtctgggatcaacgagtctgcggacatgagtattggagttact
gtcatcaaaaacaatatgataaacaatgatcttggtccagcaaccgctcaaatggcccttcagctgttcatcaaagattacaggtaca
cgtaccggtgccatagaggtgacacacaaatacaaacccgaagatcatttgaaataaagaaactgtgggagcaaacccattccaaagc
tggactgctggtctccgacggaggcccaaatttatacaacattagaaatctccacattcctgaagtctgcttgaaatgggaattaatg
gatgaggattaccaggggcgtttatgcaacccactgaacccatttgtcaaccataaagacattgaatcagtgaacaatgcagtgataa
tgccagcacatggtccagccaaaaacatggagtatgatgctgttgcaacaacacactcctggatccccaaaagaaatcgatccatctt
gaatacaagccaaagaggaatacttgaagatgaacaaatgtaccaaaagtgctgcaacttatttgaaaaattcttccccagcagttca
tacagaagaccagtcgggatatccagtatggtggaggctatggtttccagagcccgaattgatgcacgaattgatttcgaatctggaa
ggataaagaaagaggagttcactgagatcatgaaaatttgcagtacaatcgaggaacttcggagacagaagggttctggcgccaccaa
cttctccctgctgaagcaggctggcgatgtggaggagaaccctgggcccatggagacagacaccctgctgctgtgggtgctgctgctt
tgggtgcctggatctacaggagatatggcccaagtgcaactggtggaaacaggcggcggcttagtgcaacctggaggcagccttagac
tgagctgtacagctagcggatttaccttcagcatgcacgccatgacctggtacagacaggcccccggaaaacagagagagctggtggc
tgtgattaccagccacggagatagagccaactacaccgacagcgtgagaggcagattcaccatcagcagagacaacaccaagaacatg
gtgtacctgcagatgaacagcctgaagcccgaggacaccgccgtgtattattgcaatgtgcccagatacgacagctggggccaaggca
cacaagtgacagtgagcagcggaggactgcccgaaactggaggagaacaaaaactgatcagcgaggaggacctgtaa
PA-5dxw
(SEQ ID NO: 16)
atggaagattttgtgcgacaatgcttcaatccgatgattgtcgagcttgcggaaaaggcaatgaaagagtatggagaggacctgaaaa
tcgaaacaaacaaatttgcagcaatatgcactcacttggaagtgtgcttcatgtattcagattttcacttcatcgatgagcaaggcga
gtcaatagtcgtagaacttggcgatccaaatgcacttttgaagcacagatttgaaataatcgagggaagagatcgcacaatagcctgg
acagtaataaacagtatttgcaacactacaggggctgagaaaccaaagtttctaccagatttgtatgattacaagaagaatagattca
tcgaaattggagtaacaaggagagaagttcacatatactatctggaaaaggccaataaaattaaatctgagaagacacacatccacat
tttctcattcactggggaggaaatggccacaaaggccgactacactctcgatgaagaaagcagggctaggatcaaaaccaggctattc
accataagacaagaaatggctagcagaggcctctgggattcctttcgtcagtccgagagaggcgaagagacaattgaagaaagatttg
aaatcacaggaacaatgcgcaagcttgccgaccaaagtctcccgccaaacttctccagccttgaaaaatttagagcctatgtggatgg
attcgaaccgaacggctacattgagggcaagctttctcaaatgtccaaagaagtaaatgctagaattgaaccttttttgaaatcaaca
ccacgaccacttagacttccggatgggcctccctgttctcagcggtccaaattcctgctgatggatgccttaaaattaagcattgagg
acccaagtcatgagggagaggggataccgctatatgatgcaatcaaatgcatgagaacattctttggatggaaggaacccaatgttgt
taaaccacacgaaaagggaataaatccaaattatcttctgtcatggaagcaagtactggcagaactgcaggacattgagaatgaggag
aaaattccaaggactaaaaatatgaagaaaacgagtcagttaaagtgggcacttggtgagaacatggcaccagaaaaggtagactttg
acgattgtaaagatgtaggcgatttgaagcaatatgatagtgatgaaccagaattgaggtcgcttgcaagttggattcagaatgagtt
caacaaggcatgtgaactgaccgattcaagctggatagagctcgatgagattggagaagatgcggctccaattgaacacattgcaagc
atgagaaggaattatttcacagcagaggtgtctcattgcagagccacagaatacataatgaagggggtgtacatcaatactgccttgc
ttaatgcatcctgtgcagcaatggatgatttccaattaattccaatgataagcaagtgtagaactaaggagggaaggcgaaagaccaa
tttgtacggtttcatcataaaaggaagatcccacttaaggaatgacaccgatgtggtaaactttgtgagcatggagttttccctcact
gacccaagacttgaaccacacaaatgggagaagtactgtgttcttgaggtaggagatatgcttctaagaagtgccataggccatgtgt
caaggcctatgttcttgtatgtgaggacaaatggaacctcaaaaattaaaatgaaatgggggatggaaatgaggcgttgcctccttca
gtcacttcaacaaatcgagagtatgattgaagctgagtcctctgtcaaggagaaagacatgaccaaagagttctttgaaaacaaatca
gaaacatggcccgttggagagtcccccaaaggagtggaggaaggttccattgggaaggtctgcagaactttattggcaaagtcggtat
tcaacagcttgtatgcatctccacaactagaaggattttcagctgaatcaagaaaactgcttcttatcgttcaggctcttagggacaa
cctggaacctgggacctttgatcttggggggctatatgaagcaattgaggagtgcctgattaatgatccctgggttttgcttaacgcc
agctggtttaattcttttttgacgcacgcgctatcaggttctggcgccaccaacttctccctgctgaagcaggctggcgatgtggagg
agaaccctgggcccatggagacagacaccctgctgctgtgggtgctgctgctttgggtgcctggatctacaggagatatggcccaagt
gcaactggtggaaacaggcggcggcttagtgcaacctggaggcagccttagactgagctgtacagctagcggatttaccttcagcatg
cacgccatgacctggtacagacaggcccccggaaaacagagagagctggtggctgtgattaccagccacggagatagagccaactaca
ccgacagcgtgagaggcagattcaccatcagcagagacaacaccaagaacatggtgtacctgcagatgaacagcctgaagcccgagga
caccgccgtgtattattgcaatgtgcccagatacgacagctggggccaaggcacacaagtgacagtgagcagcggaggactgcccgaa
actggaggagaacaaaaactgatcagcgaggaggacctgtaa
PB2-5e03
(SEQ ID NO: 17)
agcgaaagcaggtcaattatattcaatatggaaagaataaaagaactaaggaatctaatgtcgcagtctcgcactcgcgagatactca
caaaaaccaccgtggaccatatggccataatcaagaagtacacatcaggaagacaggagaagaacccagcacttaggatgaaatggat
gatggcaatgaaatatccaattacagcagacaagaggataacggaaatgattcctgagagaaatgagcagggacaaactttatggagt
aaaatgaatgacgccggatcagaccgagtgatggtatcacctctggctgtgacatggtggaataggaatggaccagtgacaagtacag
ttcattatccaaaaatctacaaaacttattttgaaaaagtcgaaaggttaaaacatggaacctttggccctgtccattttagaaacca
agtcaaaatacgtcgaagagttgacataaatcctggtcatgcagatctcagtgccaaagaggcacaggatgtaatcatggaagttgtt
ttccctaacgaagtgggagccaggatactaacatcggaatcgcaactaacgacaaccaaagagaagaaagaagaactccagggttgca
aaatttctcctctgatggtggcatacatgttggagagagaactggtccgcaaaacgagattcctcccagtggctggtggaacaagcag
tgtgtacattgaagtgttgcatttgacccaaggaacatgctgggaacagatgtacactccaggaggggaggcgaggaatgatgatgtt
gatcaaagcttaattattgctgctagaaacatagtaagaagagccacagtatcagcagatccactagcatctttattggagatgtgcc
acagcacgcagattggtggaataaggatggtaaacatccttaggcagaacccaacagaagagcaagccgtggatatttgcaaggctgc
aatgggactgagaattagctcatccttcagttttggtggattcacatttaagagaacaagcggatcatcagtcaagagagaggaagag
gtgcttacgggcaatcttcagacattgaagataagagtgcatgagggatatgaagagttcacaatggttgggagaagagcaacagcta
tactcagaaaagcaaccaggagattgattcagctgatagtgagtgggagagacgaacagtcgattgccgaagcaataattgtggccat
ggtattttcacaagaggattgtatgataaaagcagttagaggtgacctgaatttcgtcaatagggcgaatcagcgattgaatcccatg
caccaacttttgagacattttcagaaggatgcaaaggtgctctttcaaaattggggaattgaatccatcgacaatgtgatgggaatga
tcgggatattgcccgacatgactccaagcaccgagatgtcaatgagaggagtgagaatcagcaaaatgggggtagatgagtattccag
cgcggagaagatagtggtgagcattgaccgttttttgagagttagggaccaacgtgggaatgtactactgtctcccgaggagatcagt
gaaacacagggaacagagaaactgacaataacttactcatcgtcaatgatgtgggagattaatggtcctgaatcagtgttggtcaata
cctatcagtggatcatcagaaactgggaaactgttaaaattcagtggtcccagaatcctacaatgctgtacaataaaatggaatttga
gccatttcagtctttagttccaaaggccgttagaggccaatacagtgggtttgtgagaactctgttccaacaaatgagggatgtgctt
gggacatttgataccgctcagataataaaacttcttcccttcgcagccgctccaccaaagcaaagtagaacgcagttctcctcattga
ctataaatgtgaggggatcaggaatgagaatacttgtaaggggcaattctccagtattcaactacaacaagaccactaaaagactcac
agttctcggaaaggatgctggccctttaactgaagacccagatgaaggcacagctggagttgagtccgcagttctgagaggattcctc
attctgggcaaagaagacaggagatatggaccagcattaagcataaatgaactgagcaaccttgcgaaaggagagaaggctaatgtgc
taattgggcaaggagacgtggtactagtgatgaagaggaagagaaatagctctatcttgacggattcacaaacggcaactaagaggat
ccgtatggctattaacggttctggcgccaccaacttctccctgctgaagcaggctggcgatgtggaggagaaccctgggcccatggag
acagacaccctgctgctgtgggtgctgctgctgtgggtgcctggaagcacaggagatatggctcaagtgcagctggtggagagcggcg
gaggactggctcaacctggaggaagccttagactgagctgtgctgctagcggaagcaccatcagcagcgtggctgtgggatggtatag
acagacccccggcaatcagagagagtgggtggctacaagcagcacaagcagcacaacagccacctatgccgatagcgtgaagggaaga
ttcaccatcagcagagacaacgccaagaacaccatctacctgcagatgaacagcctgaagcccgaggacaccgccgtttattattgca
aaaccggcctgaccaactggggcagaggcacacaagtgacagtgagcagcggaggcctgcctgaaacaggaggagattataaagatga
cgatgacaagtaagcgaaaggagagaaggctaatgtgctaattgggcaaggagacgtggtgttggtaatgaaacggaaacggaactct
agcatacttactgacagccagacagcgaccaaaagaattcggatggccatcaattagtgtcgaatagtttaaaaacgaccttgtttct
act
PB1-5e03
(SEQ ID NO: 18)
atggatgtcaatccgactttacttttcttaaaagtgccagcacaaaatgctataagcacaactttcccttatactggagaccctcctt
acagccatgggacaggaacaggatacaccatggatactgtcaacaggacacatcagtactcagaaaggggaagatggacaacaaacac
cgaaactggagcaccgcaactcaacccgattgatgggccactgccagaagacaatgaaccaagtggttatgcccaaacagattgtgta
ttggaagcaatggccttccttgaggaatcccatcctggtatctttgagacctcgtgtcttgaaacgatggaggttgttcagcaaacac
gagtggacaagctgacacaaggccgacagacctatgactggactctaaataggaaccagcctgctgcaacagcattggccaacacaat
agaagtgttcagatcaaatggcctcacggccaatgaatctggaaggctcatagacttccttaaggatgtaatggagtcaatgaacaaa
gaagaaatggagatcacaactcattttcagagaaagagacgagtgagagacaatatgactaagaaaatggtgacacagagaacaatag
gtaaaaggaagcagagattgaacaaaaggagttatctaattagggcattaaccctgaacacaatgaccaaagatgctgagagagggaa
gctaaaacggagagcaattgcaaccccagggatgcaaataagggggtttgtatactttgttgagacactagcaaggagtatatgtgag
aaacttgaacaatcaggattgccagttggaggcaatgagaagaaagcaaagttggcaaatgttgtaaggaagatgatgaccaattctc
aggacactgaaatttctttcaccatcactggagataacaccaaatggaacgaaaatcagaaccctcggatgtttttggccatgatcac
atatataaccagaaatcagcccgaatggttcagaaatgttctaagtattgctccaataatgttctcaaacaaaatggcgagactggga
aaggggtacatgtttgagagcaagagtatgaaaattagaactcaaatacctgcagaaatgctagcaagcatcgatttgaaatacttca
atgattcaactagaaagaagattgaaaaaatccggccgctcttaatagatgggactgcatcattgagccctggaatgatgatgggcat
gttcaatatgttaagtactgtattaggcgtctccatcctgaatcttggacaaaagagacacaccaagactacttactggtgggatggt
cttcaatcttctgatgattttgctctgattgtgaatgcacccaatcatgaagggattcaagccggagtcaacaggttttatcgaacct
gtaagctacttggaattaatatgagcaagaaaaagtcttacataaacagaacaggtacatttgaattcacaagttttttctatcgtta
tgggtttgttgccaatttcagcatggagcttcccagctttggggtgtctgggatcaacgagtctgcggacatgagtattggagttact
gtcatcaaaaacaatatgataaacaatgatcttggtccagcaaccgctcaaatggcccttcagctgttcatcaaagattacaggtaca
cgtaccggtgccatagaggtgacacacaaatacaaacccgaagatcatttgaaataaagaaactgtgggagcaaacccattccaaagc
tggactgctggtctccgacggaggcccaaatttatacaacattagaaatctccacattcctgaagtctgcttgaaatgggaattaatg
gatgaggattaccaggggcgtttatgcaacccactgaacccatttgtcaaccataaagacattgaatcagtgaacaatgcagtgataa
tgccagcacatggtccagccaaaaacatggagtatgatgctgttgcaacaacacactcctggatccccaaaagaaatcgatccatctt
gaatacaagccaaagaggaatacttgaagatgaacaaatgtaccaaaagtgctgcaacttatttgaaaaattcttccccagcagttca
tacagaagaccagtcgggatatccagtatggtggaggctatggtttccagagcccgaattgatgcacgaattgatttcgaatctggaa
ggataaagaaagaggagttcactgagatcatgaaaatttgcagtacaatcgaggaacttcggagacagaagggttctggcgccaccaa
cttctccctgctgaagcaggctggcgatgtggaggagaaccctgggcccatggagacagacaccctgctgctgtgggtgctgctgctg
tgggtgcctggaagcacaggagatatggctcaagtgcagctggtggagagcggcggaggactggctcaacctggaggaagccttagac
tgagctgtgctgctagcggaagcaccatcagcagcgtggctgtgggatggtatagacagacccccggcaatcagagagagtgggtggc
tacaagcagcacaagcagcacaacagccacctatgccgatagcgtgaagggaagattcaccatcagcagagacaacgccaagaacacc
atctacctgcagatgaacagcctgaagcccgaggacaccgccgtttattattgcaaaaccggcctgaccaactggggcagaggcacac
aagtgacagtgagcagcggaggcctgcctgaaacaggaggagattataaagatgacgatgacaagtaa
PB1-5e03
(SEQ ID NO: 19)
atggaagattttgtgcgacaatgcttcaatccgatgattgtcgagcttgcggaaaaggcaatgaaagagtatggagaggacctgaaaa
tcgaaacaaacaaatttgcagcaatatgcactcacttggaagtgtgcttcatgtattcagattttcacttcatcgatgagcaaggcga
gtcaatagtcgtagaacttggcgatccaaatgcacttttgaagcacagatttgaaataatcgagggaagagatcgcacaatagcctgg
acagtaataaacagtatttgcaacactacaggggctgagaaaccaaagtttctaccagatttgtatgattacaagaagaatagattca
tcgaaattggagtaacaaggagagaagttcacatatactatctggaaaaggccaataaaattaaatctgagaagacacacatccacat
tttctcattcactggggaggaaatggccacaaaggccgactacactctcgatgaagaaagcagggctaggatcaaaaccaggctattc
accataagacaagaaatggctagcagaggcctctgggattcctttcgtcagtccgagagaggcgaagagacaattgaagaaagatttg
aaatcacaggaacaatgcgcaagcttgccgaccaaagtctcccgccaaacttctccagccttgaaaaatttagagcctatgtggatgg
attcgaaccgaacggctacattgagggcaagctttctcaaatgtccaaagaagtaaatgctagaattgaaccttttttgaaatcaaca
ccacgaccacttagacttccggatgggcctccctgttctcagcggtccaaattcctgctgatggatgccttaaaattaagcattgagg
acccaagtcatgagggagaggggataccgctatatgatgcaatcaaatgcatgagaacattctttggatggaaggaacccaatgttgt
taaaccacacgaaaagggaataaatccaaattatcttctgtcatggaagcaagtactggcagaactgcaggacattgagaatgaggag
aaaattccaaggactaaaaatatgaagaaaacgagtcagttaaagtgggcacttggtgagaacatggcaccagaaaaggtagactttg
acgattgtaaagatgtaggcgatttgaagcaatatgatagtgatgaaccagaattgaggtcgcttgcaagttggattcagaatgagtt
caacaaggcatgtgaactgaccgattcaagctggatagagctcgatgagattggagaagatgcggctccaattgaacacattgcaagc
atgagaaggaattatttcacagcagaggtgtctcattgcagagccacagaatacataatgaagggggtgtacatcaatactgccttgc
ttaatgcatcctgtgcagcaatggatgatttccaattaattccaatgataagcaagtgtagaactaaggagggaaggcgaaagaccaa
tttgtacggtttcatcataaaaggaagatcccacttaaggaatgacaccgatgtggtaaactttgtgagcatggagttttccctcact
gacccaagacttgaaccacacaaatgggagaagtactgtgttcttgaggtaggagatatgcttctaagaagtgccataggccatgtgt
caaggcctatgttcttgtatgtgaggacaaatggaacctcaaaaattaaaatgaaatgggggatggaaatgaggcgttgcctccttca
gtcacttcaacaaatcgagagtatgattgaagctgagtcctctgtcaaggagaaagacatgaccaaagagttctttgaaaacaaatca
gaaacatggcccgttggagagtcccccaaaggagtggaggaaggttccattgggaaggtctgcagaactttattggcaaagtcggtat
tcaacagcttgtatgcatctccacaactagaaggattttcagctgaatcaagaaaactgcttcttatcgttcaggctcttagggacaa
cctggaacctgggacctttgatcttggggggctatatgaagcaattgaggagtgcctgattaatgatccctgggttttgcttaacgcc
agctggtttaattcttttttgacgcacgcgctatcaggttctggcgccaccaacttctccctgctgaagcaggctggcgatgtggagg
agaaccctgggcccatggagacagacaccctgctgctgtgggtgctgctgctgtgggtgcctggaagcacaggagatatggctcaagt
gcagctggtggagagcggcggaggactggctcaacctggaggaagccttagactgagctgtgctgctagcggaagcaccatcagcagc
gtggctgtgggatggtatagacagacccccggcaatcagagagagtgggtggctacaagcagcacaagcagcacaacagccacctatg
ccgatagcgtgaagggaagattcaccatcagcagagacaacgccaagaacaccatctacctgcagatgaacagcctgaagcccgagga
caccgccgtttattattgcaaaaccggcctgaccaactggggcagaggcacacaagtgacagtgagcagcggaggcctgcctgaaaca
ggaggagattataaagatgacgatgacaagtaa

### Specific Models for Carrying Out the Present Invention

The embodiments of the present invention will be described in detail below in conjunction with the examples, but those skilled in the art will understand that the following examples are only used to illustrate the present invention and should not be regarded as limiting the scope of the present invention. If no specific conditions were specified in the examples, the experiments were carried out according to conventional conditions or conditions recommended by the manufacturers. The reagents or instruments used without indicating the manufacturer were all conventional products that could be purchased commercially.

### Preparation Example 1: Construction of Influenza Virus Gene Vector Containing Site-Directed Mutagenesis

### (1) Construction of Plasmid for Rescue of Wild-Type Influenza Virus WSN (the Wild-Type Virus Corresponding to PTC Virus was A/WSN/1933, Briefly Referred to as WSN).

The genes of each gene fragment of the influenza virus were obtained through full gene synthesis based on the gene sequence of influenza virus A/WSN/1933 published by PubMed. The gene sequences were shown in SEQ ID NOs: 1-8. Then, they were ligated to pHH21, pCDNA 3 (neo) or pcAAGGS/MCS vectors to obtain 12 plasmids of the rescued wild-type influenza virus WSN. The names and compositions of the obtained plasmids were shown in Table 1.

TABLE 1

| | | | | Sequence |
|---|---|---|---|---|
| Plasmid | | Key | Restriction enzyme | number of |
| name | Vector used | gene | cutting site | key gene |
| Ben1 | PHH21 | PB2 | BsmBI | SEQ ID NO: 1 |
| Ben2 | PHH21 | PB1 | BsmBI | SEQ ID NO: 2 |
| Ben3 | PHH21 | PA | BsmBI | SEQ ID NO: 3 |
| Ben4 | PHH21 | HA | BsmBI | SEQ ID NO: 4 |
| Ben5 | PHH21 | NP | BsmBI | SEQ ID NO: 5 |
| Ben6 | PHH21 | NA | BsmBI | SEQ ID NO: 6 |
| Ben7 | PHH21 | M | BsmBI | SEQ ID NO: 7 |
| Ben8 | PHH21 | NS | BsmBI | SEQ ID NO: 8 |
| Ben9 | pcDNA3 (neo) | PB2 | EcoRI | SEQ ID NO: 1 |
| Ben10 | pcDNA3 (neo) | PB1 | EcoRI | SEQ ID NO: 2 |
| Ben11 | pcDNA3 (neo) | PA | EcoRI | SEQ ID NO: 3 |
| Ben12 | pcAGGS/MCS | NP | EcoRI | SEQ ID NO: 5 |

### (2) Design of Site-Directed Mutation Sites

The amino acid sites on PB2, PB1, PA and NP proteins, which had high conservativeness and were unable to restore mutation, were selected for mutation, and according to the resolved crystal structure of influenza virus HA protein (PDB: 1RVT), the amino acid sites that were close to the solvent side of the protein were selected for mutation. The mutation sites selected on each protein were shown in Table 2.

TABLE 2

| Plasmid name | Amino acid site | Plasmid name | Amino acid site |
|---|---|---|---|
| Ben1-1 | K33 | Ben4-9 | C84 |
| Ben2-1 | R52 | Ben4-10 | S86 |
| Ben3-1 | R266 | Ben4-11 | S92 |
| Ben5-1 | D101 | Ben4-12 | S126 |
| Ben4-1 | S53 | Ben4-13 | E132 |
| Ben4-2 | K57 | Ben4-14 | P135 |
| Ben4-3 | K62 | Ben4-15 | G147 |
| Ben4-4 | I64 | Ben4-16 | K170 |
| Ben4-5 | A65 | Ben4-17 | K176 |
| Ben4-6 | L67 | Ben4-18 | N179 |
| Ben4-7 | K71 | Ben4-19 | S201 |
| Ben4-8 | P82 | Ben4-20 | I256 |

### (3) Construction of Mutation Vector

Ben2, Ben3, Bne4, and Ben5 were used as plasmid templates, respectively, and the amino acid codons at the selected sites on each protein were mutated to amber stop codon TAG using a site-directed mutation kit (QuikChange® Lightning Site-Directed Mutagenesis Kits, Catalog #210518) according to its instructions. The mutation was successful after sequencing verification. The constructed mutation vectors were shown in Table 2.

### Preparation Example 2: Construction of Influenza Virus Gene Vectors Containing Foreign Genes

### (1) Construction of Ben1-Gluci, Ben2-Gluci, and Ben3-Gluci Plasmids

Taking Ben1-Gluci as an example, the construction process of Ben1-Gluci, Ben2-Gluci, and Ben3-Gluci was described. The Ben1 vector was subjected to reverse PCR using the pHH21-F and pHH21-R primer pairs in Table 3 with a high-fidelity PCR enzyme (NEB, M0541S), and the linearized vector was obtained after gel recovery. The PB2 gene of influenza virus A/WSN/1933 was used as a template, and Gaussia luciferase sequence (abbreviated as Gluci) was inserted into its C-terminus, and the sequence was synthesized by BGI. The synthesized PB2-Gluci sequence (SEQ ID NO: 11) was subjected to PCR using the PB2-Gluci-F and PB2-Gluci-R primers in Table 3, and then subjected to gel recovery to obtain the PB2-Gluci sequence with homology arm. Then, the sequence was homologously recombined with the linearized vector under the action of homologous recombinase (Bomaide, CL 117), and transformed, and single clones were selected and shaken. After sequencing the bacterial solution sample, it was compared with the whole genome sequence to screen out the positive single clone PB2-Gluci plasmid Ben1-Gluci with reliable sequence. Finally, the plasmid was extracted with an endotoxin-free plasmid extraction kit (Promega, A2393). The obtained plasmid could be used to rescue the recombinant virus.

Similarly, the PB1 and PA genes of influenza virus A/WSN/1933 were used as templates, and the Ben2-Gluci and Ben3-Gluci plasmids as shown in Table 6 were constructed and extracted according to the above method.

TABLE 3

| Primer name | Primer sequence (primer 5′-3′) | SEQ ID NO: |
|---|---|---|
| pHH21-F | aataacccggcggcccaaaatg | 21 |
| pHH21-R | cccccccaacttcggagg | 22 |
| Ben1-Gluci-F | gacctccgaagttgggggggagcgaaagcaggtcaattatattc | 23 |
| Ben1-Gluci-R | ttttgggccgccgggttattagtagaaacaaggtcgtttttaaac | 24 |
| Ben2-Gluci-F | gacctccgaagttgggggggagcgaaagcaggcaaacc | 25 |
| Ben2-Gluci-R | ttttgggccgccgggttattagtagaaacaaggcattttttcatgaag | 26 |
| Ben3-Gluci-F | gacctccgaagttgggggggagcgaaagcaggtactgattc | 27 |
| Ben3-Gluci-R | ttttgggccgccgggttattagtagaaacaaggtacttttttgg | 28 |

### (2) Construction of Ben1-5dxw, Ben2-5dxw, and Ben3-5dxw Plasmids

Taking Ben1-5dxw as an example, the construction process of Ben1-5dxw was described. The previously synthesized Ben1-Gluci was used as a vector template, and the pHH21-Ben1-F and pHH21-Ben1-R primers in Table 4 were used to perform reverse PCR with a high-fidelity PCR enzyme. The linearized vector was obtained after gel recovery. Subsequently, the nucleic acid sequence of the nanobody 5dxw (amino acid sequence was shown in SEQ ID NO: 9, 5dxw was the PDB (protein data bank) number of a protein crystal, which was a nanobody against mouse PD-L1 protein, and a reported antibody for anti-tumor use) was synthesized by BGI, and PCR and gel recovery were performed using the PB2-5dxw-F and PB2-5dxw-R primer pairs in Table 2 to obtain a sequence with homology arm. Then, it was homologously recombined with the linearized vector under the action of homologous recombinase, and transformed, and single clones were selected and shaken. After sequencing the bacterial solution sample, the positive single clone Ben1-5dxw plasmid with reliable sequence was screened out, and finally the plasmid was extracted using an endotoxin-free plasmid extraction kit. The obtained plasmid could be used to rescue recombinant virus.

Similarly, Ben2-Gluci and Ben3-Gluci plasmids were used as vector templates, and the Ben2-5dxw and Ben3-5dxw plasmids as shown in Table 6 were constructed following the construction method of Ben1-5dxw.

TABLE 4

| Primer name | Primer sequence (primer 5′-3′) | SEQ ID NO: |
|---|---|---|
| pHH21-Ben1-F | gcgaaaggagagaaggctaatgtg | 29 |
| pHH21-Ben1-R | gggcccagggttctcctc | 30 |
| PB2-5dxw-F | tggaggagaaccctgggcccatggagacagacaccctg | 31 |
| PB2-5dxw-R | ttagccttctctcctttcgcttacaggtcctcctcgctg | 32 |
| pHH21-Ben2-F | taatccagagcccgaattgatgc | 33 |
| pHH21-Ben2-R | gggcccagggttctcctc | 34 |
| PB1-5dxw-F | tggaggagaaccctgggcccatggagacagacaccctg | 35 |
| PB1-5dxw-R | gcatcaattcgggctctggattacaggtcctcctcgctg | 36 |
| pHH21-Ben3-F | taagaacctgggacctttgatcttgg | 37 |
| pHH21-Ben3-R | gggcccagggttctcctc | 38 |
| PA-5dxw-F | tggaggagaaccctgggcccatggagacagacaccctg | 39 |
| PA-5dxw-R | agatcaaaggtcccaggttcttacaggtcctcctcgctg | 40 |

### (3) Construction of Ben1-5e03, Ben2-5e03, and Ben3-5e03 Plasmids

According to the plasmid construction method in (2) above, the primers in Table 5 were used, and the 5dxw sequence was replaced with 5e03 sequence (SEQ ID NO: 10, 5e03 was the PDB (protein data bank) number of a protein crystal, which was a nanobody against mouse CTLA-4 protein, and a reported antibody for anti-tumor use) to construct the Ben1-5e03/Ben2-5e03/Ben3-5e03 series of plasmids as shown in Table 6.

TABLE 5

| Primer name | Primer sequence (primer 5′-3′) | SEQ ID NO: |
|---|---|---|
| PB2-5e03-F | tggaggagaaccctgggcccatggagacagacaccctg | 41 |
| PB2-5e03-R | ttagccttctctcctttcgcttacttgtcatcgtcatctttataatc | 42 |
| PB1-5e03-F | tggaggagaaccctgggcccatggagacagacaccctg | 43 |
| PB1-5e03-R | tcaattcgggctctggattacttgtcatcgtcatctttataatctc | 44 |
| PA-5e03-F | tggaggagaaccctgggcccatggagacagacaccctg | 45 |
| PA-5e03-R | tcaaaggtcccaggttcttacttgtcatcgtcatctttataatctc | 46 |

TABLE 6

| Plasmid name | Vector | Key gene | Sequence number of key gene |
|---|---|---|---|
| Ben1-Gluci | PHH21 | PB2-Gluci | SEQ ID NO: 11 |
| Ben2-Gluci | PHH21 | PB1-Gluci | SEQ ID NO: 12 |
| Ben3-Gluci | PHH21 | PA-Gluci | SEQ ID NO: 13 |
| Ben1-5dxw | PHH21 | PB2-5dxw | SEQ ID NO: 14 |
| Ben2-5dxw | PHH21 | PB1-5dxw | SEQ ID NO: 15 |
| Ben3-5dxw | PHH21 | PA-5dxw | SEQ ID NO: 16 |
| Ben1-5e03 | PHH21 | PB2-5e03 | SEQ ID NO: 17 |
| Ben2-5e03 | PHH21 | PB1-5e03 | SEQ ID NO: 18 |
| Ben3-5e03 | PHH21 | PA-5e03 | SEQ ID NO: 19 |

### Preparation Example 3: Preparation of Replication-Deficient Influenza Virus Capable of Delivering Foreign Gene

(1) According to the normal method of rescuing influenza viruses, the 12 plasmids (see Table 1) used for rescuing influenza viruses were transfected into mammalian stable cell line HEK293-PYL (see reference, Longlong Si et al, Generation of influenza A viruses as live but replication-incompetent virus vaccines science, 2016, 6316, 1170-1173.) that could stably express tRNA (tRNA^{Pyl}) and pyrrolysyl-RNA synthetase (tRNA^{Pyl}), except that the corresponding plasmids among the 12 plasmids were replaced by plasmids with site-directed mutations and plasmids containing foreign genes (or wild-type plasmids). 0.1 μg of each plasmid was added to each well of a six-well plate. Replacement with fresh culture medium was performed 6 hours after transfection, in which the culture medium contained 1% FBS, 2 μg/ml TPCK-trypsin, 1 mM non-natural amino acid NF-2-azidoethyloxycarbonyl-L-lysine (NAEK), and a culture medium without non-natural amino acid was used as control. The pathological changes of cells were observed, and mutants that showed pathological changes in cells cultured in the culture medium containing non-natural amino acids, but did not show pathological changes in cells cultured in the culture medium without non-natural amino acids, were used as positive mutants.

For example, Ben1-Gluci, Ben2-1, Ben3-1, Ben4-1, Ben5-1, Ben6, Ben7, Ben8, Ben9, Ben10, Ben11 and Ben12 were co-transfected into HEK293-PYL, so that the rescued influenza viruses contained the expression gene (Gluci) of Gaussia luciferase after the PB2 gene segment, and the packaging exogenous gene of TAG was introduced at the corresponding sites of the PB1, PA, NP and HA gene segments in the replication-deficient influenza viruses.
(2) When the pathological changes of the stable cell line with the rescued viruses aforementioned were completed, the cell supernatant was collected, and centrifuged at 5000 g for 10 min, and the supernatant was taken and filtered through a 0.45 μm filter membrane; the virus solution from the previous step was centrifuged in a 50 ml centrifuge tube at 10⁵ g for 2 hours, the precipitate was resuspended with 1 ml of PBS; 20% sucrose solution was added to a 15 ml centrifuge tube, the above PBS resuspension was added dropwise to the sucrose solution, and centrifugation was performed at 11×10⁴ g for 2 hours; 15 ml of PBS was added to the above precipitate, and centrifugation was performed at 11×10⁴ g for 2 hours; and the above precipitate was resuspended with PBS.

The combinations that could rescue the viruses and were dependent on non-natural amino acids were partially screened out. As shown in Table 7, the results showed that when the PB2 gene fragment was introduced into Gluci, the four sites Ben2-1, Ben3-1, Ben4-1, and Bne5-1 had the highest efficiency in rescuing the viruses; when the PB1 gene fragment was introduced into Gluci, the four sites Ben1-1, Ben3-1, Ben4-1, and Bne5-1 had the highest efficiency in rescuing the viruses; when the PA gene fragment was introduced into Gluci, the four sites Ben1-1, Ben2-1, Ben4-1, and Bne5-1 had the highest efficiency in rescuing the viruses, which were relatively ideal combinations. The inventors compared the expression amounts of Gluci in cells infected with the Glcui gene-containing replication-deficient influenza viruses rescued by the above three combinations. The results were shown in FIG. 1. When the PB2 gene was inserted into an exogenous gene such as Glcui reporter gene, the expression efficiency was the highest.

TABLE 7

| Name of | | Name of Gluci | Rescue |
|---|---|---|---|
| packaged virus | Name of plasmid introducing TAG | fusion plasmid | efficiency (%) |
| P1-1 (free of | Ben2-1, Ben3-1, Ben4-1, Bne5-1 | Ben1 | 90 |
| Gluci) | | | |
| P1Glcui-1 | Ben2-1, Ben3-1, Ben4-1, Bne5-1 | Ben1-Gluci | 87 |
| P1Glcui-2 | Ben2-1, Ben3-1, Ben4-5, Bne5-1 | Ben1-Gluci | 82 |
| P1Glcui-3 | Ben2-1, Ben3-1, Ben4-8, Bne5-1 | Ben1-Gluci | 75 |
| P1Glcui-4 | Ben2-1, Ben3-1, Ben4-13, Bne5-1 | Ben1-Gluci | 33 |
| P1Glcui-5 | Ben2-1, Ben3-1, Ben4-15, Bne5-1 | Ben1-Gluci | 15 |
| P1Glcui-6 | Ben2-1, Ben3-1, Ben4-19, Bne5-1 | Ben1-Gluci | 54 |
| P2-1 | Ben1-1, Ben3-1, Ben4-1, Bne5-1 | Ben2 | 75 |
| P2Glcui-1 | Ben1-1, Ben3-1, Ben4-1, Bne5-1 | Ben2-Gluci | 65 |
| P2Glcui-2 | Ben1-1, Ben3-1, Ben4-5, Bne5-1 | Ben2-Gluci | 55 |
| P2Glcui-3 | Ben1-1, Ben3-1, Ben4-8, Bne5-1 | Ben2-Gluci | 58 |
| P2Glcui-4 | Ben1-1, Ben3-1, Ben4-13, Bne5-1 | Ben2-Gluci | 62 |
| P2Glcui-5 | Ben1-1, Ben3-1, Ben4-15, Bne5-1 | Ben2-Gluci | 10 |
| P2Glcui-6 | Ben1-1, Ben3-1, Ben4-19, Bne5-1 | Ben2-Gluci | 15 |
| P3-1 | Ben1-1, Ben2-1, Ben4-1, Bne5-1 | Ben3 | 80 |
| P3Glcui-1 | Ben1-1, Ben2-1, Ben4-1, Bne5-1 | Ben3-Gluci | 56 |
| P3Glcui-2 | Ben1-1, Ben2-1, Ben4-5, Bne5-1 | Ben3-Gluci | 63 |
| P3Glcui-3 | Ben1-1, Ben2-1, Ben4-8, Bne5-1 | Ben3-Gluci | 62 |
| P3Glcui-4 | Ben1-1, Ben2-1, Ben4-13, Bne5-1 | Ben3-Gluci | 71 |
| P3Glcui-5 | Ben1-1, Ben2-1, Ben4-15, Bne5-1 | Ben3-Gluci | 78 |
| P3Glcui-6 | Ben1-1, Ben2-1, Ben4-19, Bne5-1 | Ben3-Gluci | 26 |

Description of Table 7: Taking the packaged P1Glcui-1 virus as an example, among the five plasmids as listed, Ben2-1, Ben3-1, Ben4-1, and Bne5-1 were plasmids for introducing tags (see Table 2), Ben1-Gluci was a plasmid for introducing Gluci at the truncation PB2 (see Table 6), and the other seven plasmids were the remaining wild-type plasmids for packaging viruses, namely Ben6, Ben7, Ben8, Bne9, Bne10, Ben11, and Ben12.

Similarly, according to the preparation method of P1Gluci-1, the Ben1-Gluci plasmid was replaced with the Ben1-5dxw plasmid or the Ben1-5e03 plasmid when rescuing the virus to obtain the replication-deficient influenza virus P15dxw or P15e03 that could express nanobodies.

### Preparation Example 4: Preparation of Armed Influenza Virus Tumor Vaccine

The compound of Formula I was purchased from Shanghai Taopu Biotechnology Co., Ltd., and the compound of Formula II was purchased from Shenggong Biotechnology Co., Ltd. OVA1 in the compound of Formula I is an amino acid sequence, and the specific amino acid sequence was shown in Table 8.

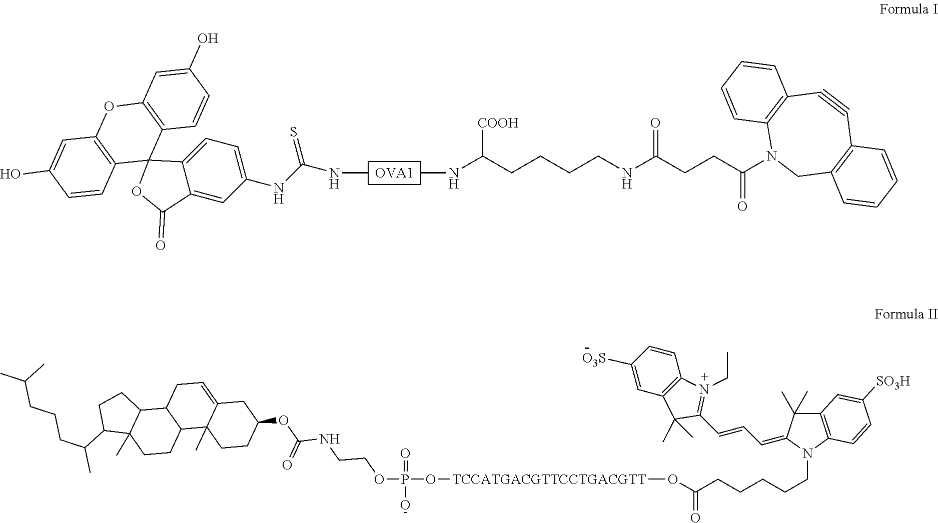

CpG adjuvant generally referred to TCCATGACGTTCCTGACGTT (SEQ ID NO: 20) sequence (as included in Formula II), and the modification group (cholesterol) on the left side of the sequence was conducive to the anchoring of the CpG adjuvant on the viral envelope, and the right side of the sequence was a fluorescent group, which was used to subsequently verify that the adjuvant was anchored on the viral envelope. The CpG adjuvant was a TLR9 agonist and one of the commonly used adjuvants in vaccines to enhance the function of DC cells.

If not otherwise specified, CpG or CpG adjuvant in this example referred to chemically modified CpG (i.e., the compound of Formula II).

The feasibility of covalent coupling of replication-deficient influenza virus with tumor antigen peptide and membrane insertion modification of immune adjuvant were verified. Taking the packaged virus P1-1 in Table 7 as an example, 108 pfu/ml P1-1 was mixed with the compound of Formula I and the compound of Formula II containing the OVA1 sequence in Table 8, and click chemistry reaction and membrane insertion modification were performed, in which the final concentration of the compound of Formula I was 100 M and the final concentration of the compound of Formula II was 10 μM, the reaction was carried out at 4° C. under gently shaking for 2 hours. Then, the unreacted compound of Formula I and compound of Formula II were removed by size-exclusion chromatography (HiTrap Capto Core 700, GE Healthcare). The virus particle elution peak was collected using a 100 KDa centrifugal filter unit (Millipore), concentrated, and buffer exchanged into PBS buffer. The carbon-carbon triple bond in the structure of Formula I underwent a click reaction with the azide on the non-natural amino acid NAEK on the virus. The connection method of Formula I was shown below. Formula II was inserted into the viral envelope through cholesterol.

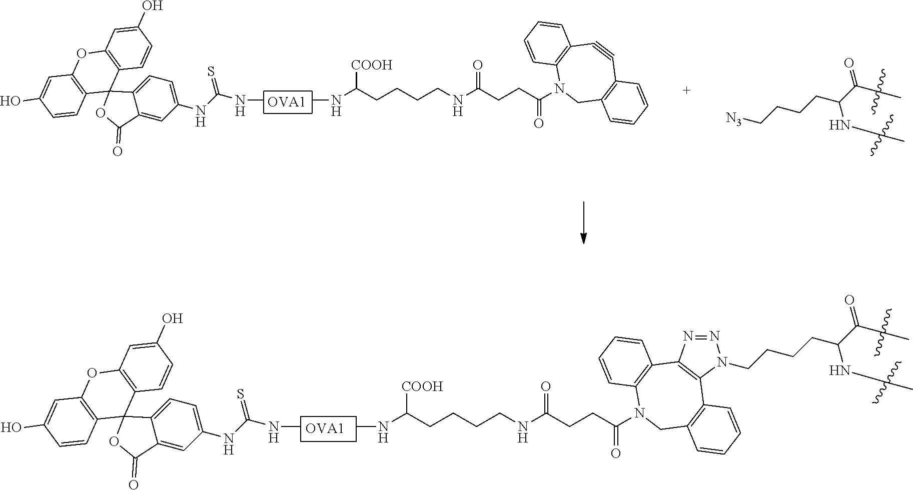

In the above reaction, NAEK already existed in an amino acid sequence, and the two wavy lines “ [symbol] ” respectively represented the carboxyl end of the previous amino acid and the amino end of the next amino acid.

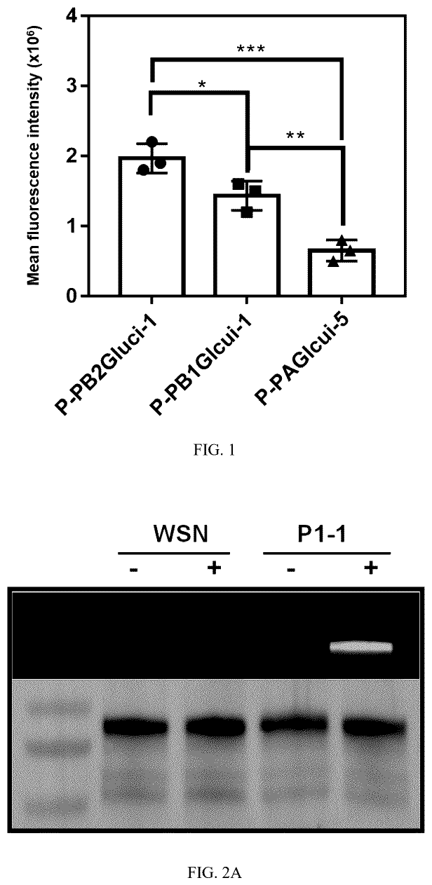

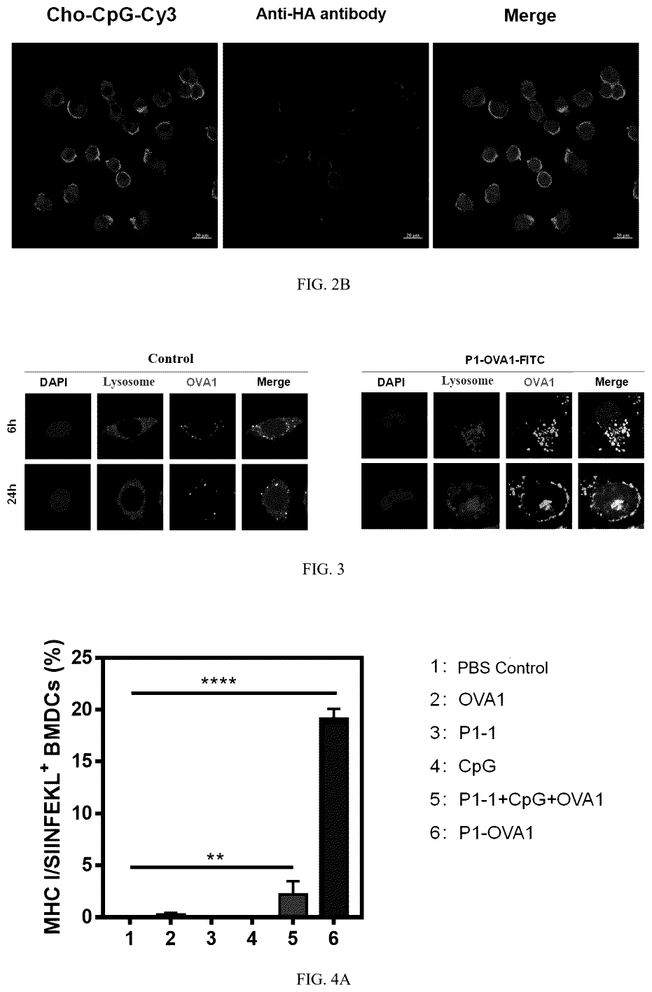

It was verified by sodium dodecylsulfate polyacrylamide gel electrophoresis (SDS-PAGE) under denaturing conditions and fluorescence imaging that OVA1 could be coupled to the influenza virus HA protein, and the results were shown in FIG. 2A. Confocal imaging was used to verify that CpG could be anchored on the influenza virus envelope (the infected cells were of lung tumor cell line A549 that simulated lung), and the results were shown in FIG. 2B. The obtained virus was named P1-OVA1-FITC.

Since the compound of Formula I and the compound of Formula II both had fluorescent groups at right side, the main purpose of this experiment was to verify that small peptides and CpG could be loaded onto the virus surface. In practice, fluorescent groups could not be comprised in the application process. It was preferred to use the compound of Formula III-1, the compound of Formula III-2, the compound of Formula III-3, the compound of Formula III-4, the compound of Formula III-5 or the compound of Formula III-6 to replace the compound of Formula I; and to use the compound of Formula IV to replace the compound of Formula II. Preferably, the antigen peptide (tumor antigen peptide) in the compound of Formula III-1 to the compound of Formula III-6 was selected from the antigen peptides in Table 8.

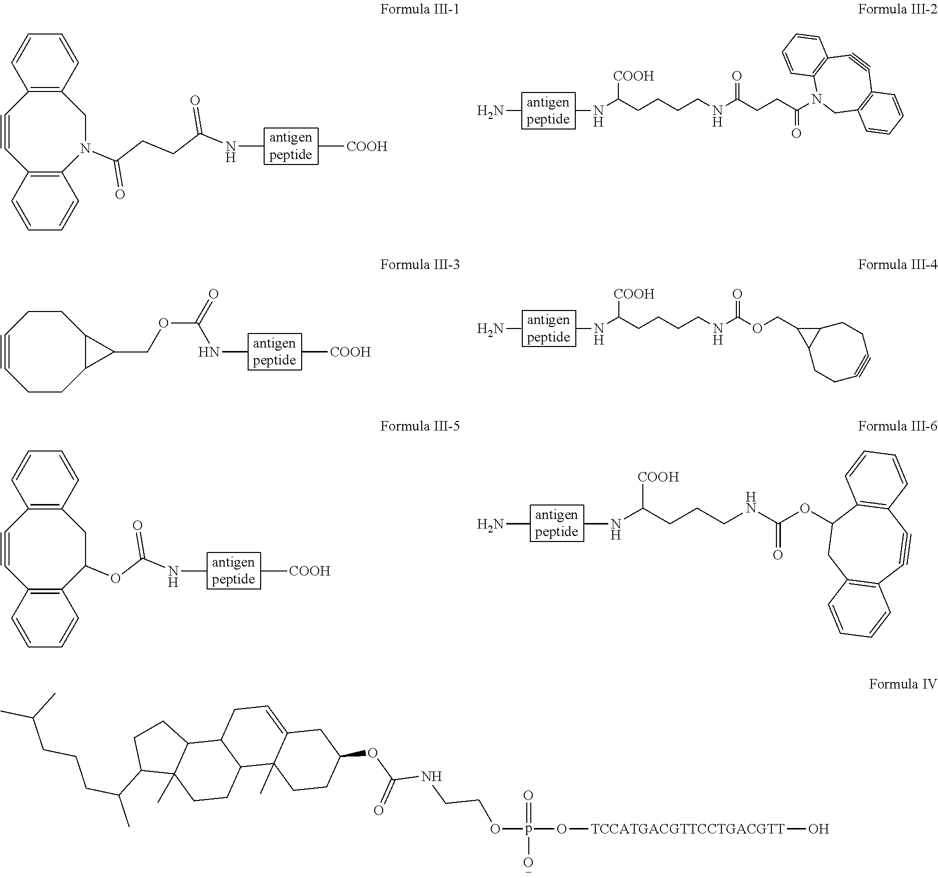

The compound of Formula III-1 to the compound of Formula III-6 (used to load the antigen peptide) was coupled to an azide group-containing hemagglutinin protein on the virus surface through a click reaction. At present, there were many groups that reacted with azide. Among the compound of Formula III-1 to the compound of Formula III-6, only two reactive groups and two connection methods of N-terminus and C-terminus were listed.

The compound of Formula IV was the compound of Formula II without the fluorescent group. Replacing Formula II with Formula IV could prepare armed influenza virus tumor vaccines, which were named P1-OVA1, P1-OVA2, P1-B16, P1-CT26, and P1-4T1, respectively.

Similarly, by using P15dxw or P15e03 virus particles to modify antigen peptides and CpG through the above method, armed influenza virus tumor vaccines carrying anti-PDL1 (5dxw) nanobody gene and anti-CTLA4 (5e03) nanobody gene could be prepared, which were named P15dxw-OVA1, P15dxw-OVA2, P15dxw-B16, P15dxw-CT26, P15dxw-4T1, P15e03-OVA1, P15e03-OVA2, P15e03-B16, P15e03-CT26 and P15e03-4T1, respectively.

TABLE 8

| Name of | | SEQ | |
|---|---|---|---|
| antigen | | ID | Name of |
| peptide | Amino acid sequence | NO: | virus |
| OVA1 | SIINFEKLTEWTSSNVMEERKIK | 47 | P1-OVA1 |
| OVA2 | ISQAVHAAHAEINEAGR | 48 | P1-OVA2 |
| gp100 | AVGALEGPRNQDWLGVPRQL | 49 | P1-B16 |
| Trp1 | EGPAFLTWHRYHLLQLERDM | 50 | |
| Trp2 | QIANCSVYDFFVWLHYYSVR | 51 | |
| CT26-M68 | VTSIPSVSNALNWKEFSFIQSTLGYVA | 52 | P1-CT26 |
| CT26-M55 | EGDPCLRSSDCIDEFCCARHFWTKICK | 53 | |
| CT26-M20 | PLLPFYPPDEALEIGLELNSSALPPTE | 54 | |
| 4T1-M32 | SHRSCSHQTSAPSPKALAHNGTPRNAI | 55 | P1-4T1 |

### Effect Example 1: Determination of Dendritic Cell (DC) Function Enhanced by Armed Influenza Viruses

The inventors co-cultured P1-OVA1-FITC with mouse bone marrow-derived dendritic cells (BMDC), the cells were taken at different culture time points, stained with lysosomes, and imaged using a laser confocal microscope to observe the distribution of FITC-labeled OVA1 antigen peptide sequence in the cells. Specifically: immature BMDC cells were adjusted to 10⁶ cells/well in 12-well plates with 1640 complete medium (the 1640 medium supplemented was added with 10% fetal bovine serum, 20 ng/ml GM-CSF, 10 ng/ml IL-4), cultured at 37° C., 5% CO₂ for 24 hours, and then 100 nM CpG and 3 M small peptide mixture or P1-OVA1-FITC with MOI=100 were added. After 6 hours or 24 hours of culture under the same culture conditions, the cultured cells were taken out for lysosome staining and imaged with a laser confocal microscope to observe the distribution of FITC-labeled OVA1 antigen peptide sequence in the cells.

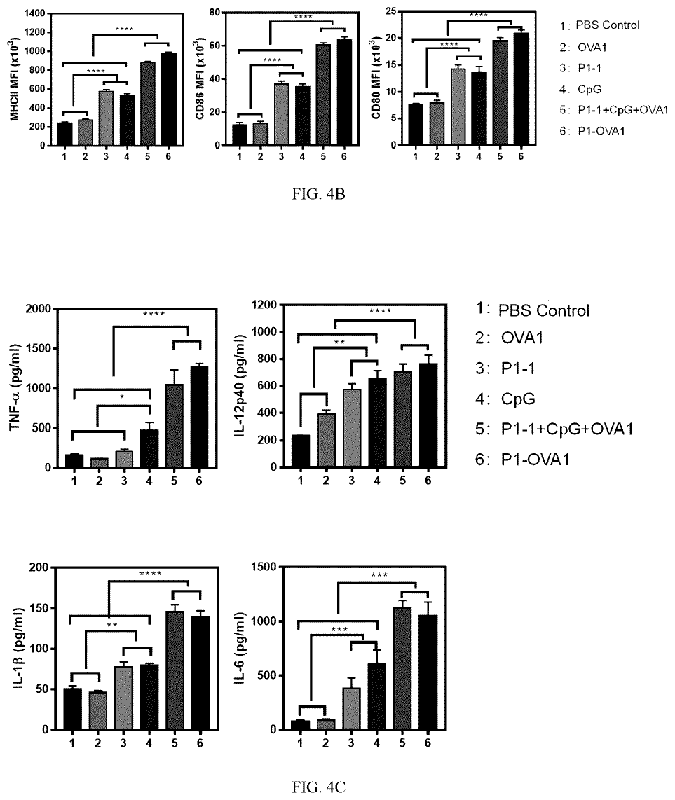

Different drugs were added during the culture process. As shown in FIG. 4A, there were six groups: 1, 2, 3, 4, 5, and 6, among them, Group 1 was the pbs control group without drug; Group 2 was the group with 3 M small peptide; Group 3 was the group with P1-1 at MOI=100; Group 4 was the group with 100 nM CpG; Group 5 was the group with 3 M small peptide, 100 nM CpG, and P1-1 at MOI=100; and Group 6 was the group with P1-OVA1 at MOI=100.

The results were shown in FIG. 3. When cultured for 6 hours, more green fluorescence signals were observed in BMDCs in the P1-OVA1-FITC group as compared with the control group (FITC-modified OVA1 sequence peptide), indicating that P1-OVA1-FITC greatly promoted the uptake of antigens by the DC cells. After 24 hours of culture, green fluorescence signals were obviously observed on the BMDC cell membrane in the P1-OVA1-FITC group as compared with the control group, indicating that OVA1 had been processed by the DC cells and presented to the surface of dendritic cells to form an epitope peptide-MHC-I complex.

The inventors detected BMDC under different drug addition conditions by flow cytometry, and the results were shown in FIG. 4A. Compared with other experimental groups, the SIINFEKL-MHCI complex could be significantly detected on the surface of BMDC cells in the P1-OVA1 group, indicating that P1-OVA1 could promote the DC cells to process tumor antigen peptides and present them to the MHCI complex on the cell surface, which was beneficial to the activation of specific T cells by DC cells, thereby producing tumor immunity effect. In addition, the MHCII, CD86 and CD80 surface markers on the surface of DC cells increased significantly, indicating that the DC cells could be activated by P1-OVA1 to produce costimulatory receptors that were beneficial to T cell activation (FIG. 4B).

At the same time, the inventors detected the secretion amounts of cytokines in the cell culture supernatant by using ELISA. The results showed that the secretion amounts of TNF-α, IL-12β, IL-1β, and IL-6 cytokines by the BMDC cells co-cultured with P1-OVA1 (Group 6) increased significantly. These cytokines were only significantly secreted when the DC cells were matured, which once again proved that P1-OVA1 had the effect of maturing DC cells (FIG. 4C).

The above data showed that P1-OVA1 could efficiently deliver the exogenous antigen peptide covalently coupled to its surface (hemagglutinin protein on the viral envelope) to the DC cells, and P1-OVA1 further activated DC cells by carrying the immune adjuvant CpG, which was more conducive to the anti-tumor effect of T cell activation and had a significant and beneficial technical effect.

### Effect Example 2: Activation of Humoral Immunity and Cellular Immunity by Armed Influenza Virus Through Respiratory Tract Infection

(1)

In order to prove that the armed influenza virus could simulate real virus and enter the lungs through the respiratory tract, verification was performed by using fluorescent label-carrying P1-OVA1-FITC. C57BL/6 mice (6 to 8 weeks old, female) were intranasally administered with P1-OVA1-FITC (10⁵ pfu per mouse) or control (15 μg of OVA1-FITC and 3 μg of CpG-Cy3 per mouse) (6 mice per group), and lung tissues were taken at 24 h and 48 h, respectively, sliced, stained, and imaged.

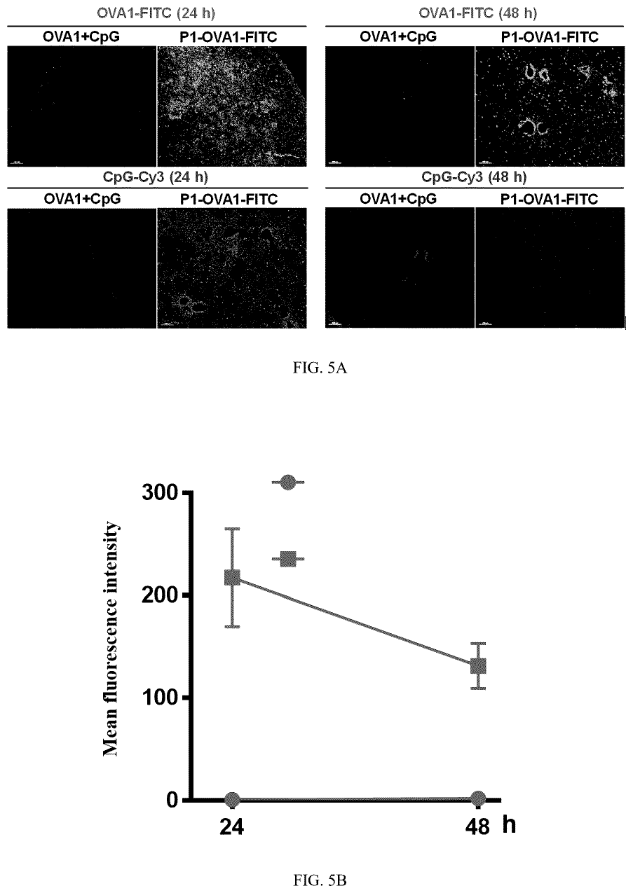

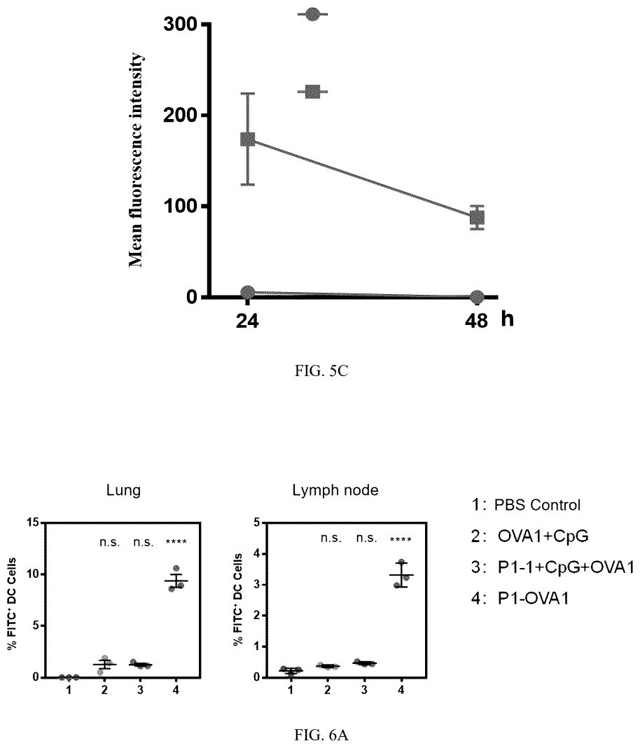

The results were shown in FIG. 5A. After 24 h and 48 h of administration, obvious green (OVA1) and red (CpG) fluorescence could be observed in the P1-OVA1-FITC group, while almost no fluorescence could be observed in the control group, indicating that the armed influenza virus could deliver exogenous peptide and adjuvant to the lungs, while the mixture of antigen peptide and adjuvant that was administered alone could hardly enter the lungs through the respiratory mucosas. The fluorescence statistics were shown in FIGS. 5B and 5C.

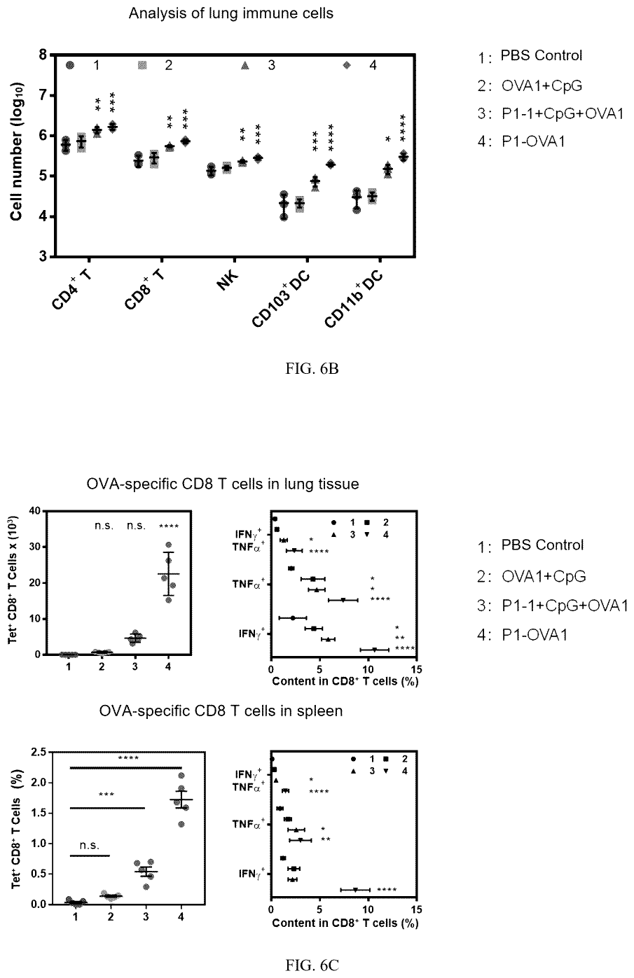

At the same time, the lung tissues and lung mediastinal lymph nodes of mice were taken 24 h after the administration, and the contents of FITC-positive DC cells (i.e., OVA1-positive) were analyzed. The results were shown in FIG. 6A. Only when OVA1 was covalently coupled to the virus, i.e., in the P1-OVA1 group, FITC-positive DC cells could be detected in the lung tissues and lung mediastinal lymph nodes, proving that the armed influenza virus could deliver exogenous peptide to DC cells, and the DC cells could migrate from the lungs to the lymph nodes to further produce immune responses.

(2)

In another set of experiments, in order to demonstrate the effects of the armed influenza virus on the proliferation and activation of immune cells in healthy mice, C57BL/6 mice (6 to 8 weeks old, female) were intranasally administered with P1-OVA1 (10⁵ pfu/25 μl per mouse), PBS control (25 μl per mouse), antigen peptide control (15 μg of OVA1+3 μg of CpG/25 μl per mouse) or virus mixed control (10⁵ pfu P1-1+15 μg of OVA1+3 μg of CpG/25 μl per mouse) (8 mice per group). Seven days after the administration, lung tissues and spleens were obtained and ground into single cell suspensions, and then immune cell subtypes were determined by flow cytometry.

As shown in FIG. 6B, the P1-OVA1 administration group and the virus mixed control group both showed significantly increased contents of CD4 T, CD8 T, NK, and DC cells in the lung tissue, and the P1-OVA1 administration group showed further increased contents of the above immune cells on the basis of the virus mixed control group (FIG. 6B), indicating that the administration of the replication-deficient influenza virus could significantly recruit immune cells in the lungs, and the armed P1-OVA1 could further increase the proportion of immune cells, which was conducive to the anti-tumor immune effect.

In addition, the results of OVA1-specific T cell detection showed that both the lungs and spleens produced a large number of OVA1-specific CD8 T cells in the P1-OVA1 administration group, and the CD8 T cells could secrete massive IFNγ and TNFα after in vitro OVA1 antigen peptide stimulation (FIG. 6C), but such obvious changes were not observed in the virus mixed control group, indicating that only by covalently coupling exogenous antigen peptide on the virus could the presentation of exogenous antigen be promoted and the body be stimulated to produce a specific immune response to the antigen peptide, while simply mixing the virus with the antigen peptide and adjuvant brought about much worse effect.

(3)

In another set of experiments, in order to demonstrate the effect of the armed influenza virus on humoral immunity in healthy mice, OVA1 was replaced with OVA2 (B cell epitope of OVA protein) to obtain P1-OVA2, and C57BL/6 mice (6 to 8 weeks old, female) were intranasally administered with P1-OVA2 (10⁵ pfu/25 μl per mouse), PBS control (25 μl per mouse), antigen peptide control (15 μg of OVA2+3 μg of CpG/25 μl per mouse), or virus mixed control (10⁵ pfu P1-1+15 μg of OVA2+3 μg of CpG/25 μl per mouse). Mouse sera were collected 21 days later, and P1-OVA2 (10⁵ pfu/25 μl per mouse), PBS control (25 μl per mouse), antigen peptide control (15 μg of OVA2+3 μg of CpG/25 μl per mouse) or virus mixed control (10⁵ pfu P1-1+15 μg of OVA2+3 μg of CpG/25 μl per mouse) (8 mice per group) were administered by nasal drops on the 22^{nd} day. Mouse sera and lung lavage fluids were collected on the 42^{nd} day to determine the contents of different antibodies.

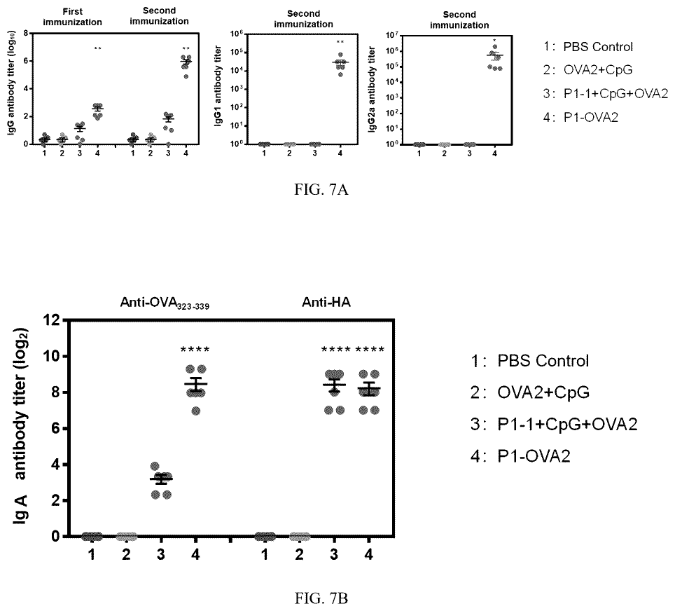

The results were shown in FIG. 7A. After the P1-OVA2 immunization of mice, IgG antibodies against OVA2 could be significantly detected. After the second immunization, the antibody titer was further increased. At the same time, large amounts of IgG1 and IgG2a antibodies could be detected after the P1-OVA2 immunization (FIG. 7A), indicating that the armed influenza virus could significantly stimulate the body to produce multiple types of antibodies against the exogenous peptide and produce strong humoral immunity.

In addition, the results of antibody detection in lung lavage fluids showed that P1-OVA2 significantly stimulated the lung mucosas to produce IgA antibodies against OVA2, and the antibody levels were comparable to the levels of HA protein antibodies of influenza itself, indicating that the armed influenza virus could significantly stimulate the body to produce mucosal immunity (FIG. 7B).

The above data showed that after the replication-deficient influenza virus was subjected to covalent coupling to the surface antigen peptide and modification and membrane-insertion of CpG adjuvant, the nasal immunization thereof had the effect of recruiting immune cells to the lungs, and could stimulate the body to produce specific cellular immunity and humoral immunity against the coupled antigen peptide.

### Effect Example 3: Experiment on Prevention of Lung Metastasis from Melanoma

The above results showed that the armed influenza virus carrying exogenous antigen peptide had a strong ability to activate DC cells and promote antigen presentation, could effectively activate lung immune cells, and could activate CD8 T cells specific to the exogenous antigen. Based on this, the role of the armed influenza virus in preventing tumor lung metastasis was investigated in this example.

C57BL/6 mice (6 to 8 weeks old, female) were immunized intranasally with P1-OVA1 (10⁵ pfu/25 μl per mouse, administered once every 7 days, twice in total), PBS control (25 μl per mouse, administered once every 7 days, twice in total), antigen peptide control (15 μg of OVA1+3 μg of CpG/25 μl per mouse, administered once every 7 days, twice in total), or virus mixed control (10⁵ pfu P1-1+15 μg of OVA1+3 μg of CpG/25 μl per mouse, administered once every 7 days, twice in total) (10 mice per group). 7 days after the second immunization, B16-F10-OVA melanoma cells (the cell line was a stable cell line that could stably express OVA protein and was prepared by lentiviral transduction of B16-F10 cells (purchased from ATCC, CRL-6475)) were injected through the tail vein (3×10⁵ cells/200 μl per mouse), and the mice were killed 21 days after the tumor inoculation. The lungs of the mice were taken to calculate the lung metastasis from melanoma, and the tumor tissues were taken, chopped and digested in a buffer containing 2 mg/ml type II and type IV collagenase (GIBCO BRL) and 0.5 mg/ml DNase (Sigma Aldrich) at 37° C. for 13 minutes to form a single cell suspension, and then the content of OVA-specific CD8 T cells in the tumor microenvironment was determined by flow cytometry.

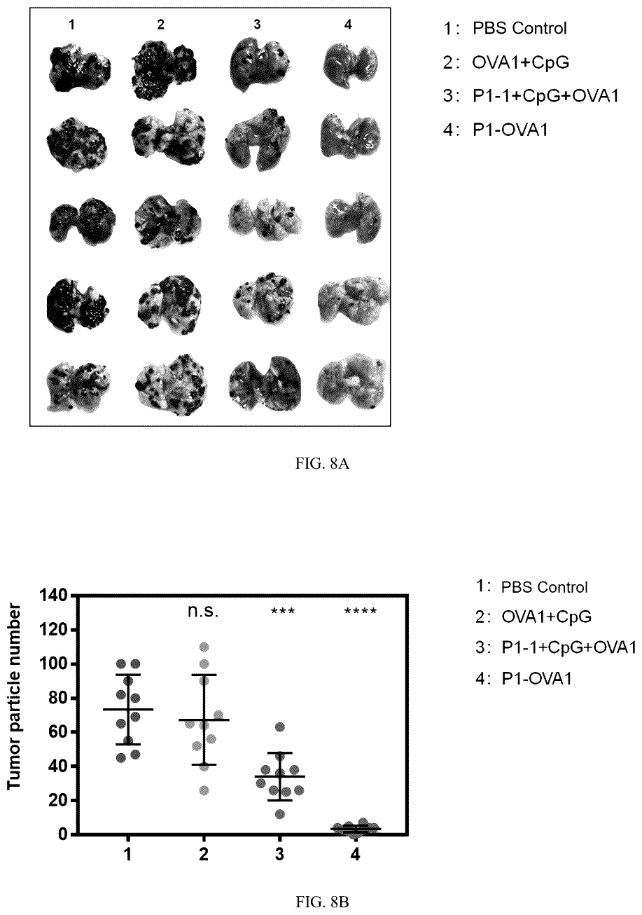

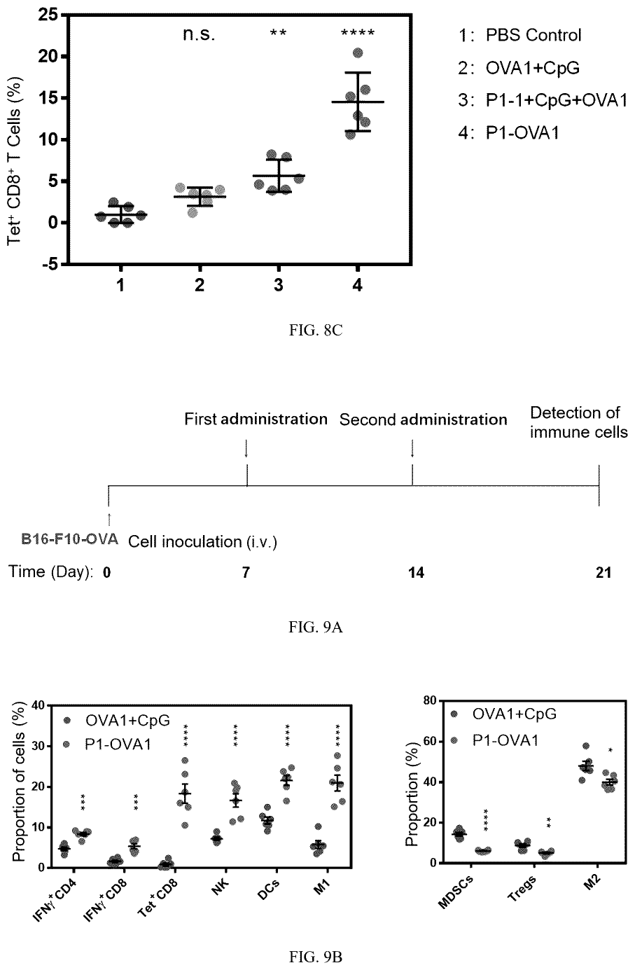

The results were shown in FIGS. 8A to 8C.

The results showed that the P1-OVA1 group showed a very outstanding ability against B16-F10-OVA lung metastasis, and the tumor load was significantly reduced (FIG. 8A, FIG. 8B). The virus mixed control group also showed a certain anti-tumor ability, which once again confirmed that the replication-deficient influenza virus could activate the lung immune system, reflecting its superiority as a tumor vaccine. Tumor-specific CD8 T cells in tumor tissues played an important role in tumor immunity. The analysis of T lymphocytes in mouse lung tumor nodules showed that the administration of PBS or antigen peptide mixture could not stimulate the immune system to produce CD8 T cells targeting the antigen peptide. Similarly, the virus mixed control group only produced a small number of CD8 T cells targeting the antigen peptide, while the P1-OVA1 group could induce a large number of CD8 T cells targeting the antigen peptide to enter into the tumor tissues (FIG. 8C).

### Effect Example 4: Regulation of Tumor Microenvironment by Armed Influenza Virus

Influenza virus is a natural immunogenic substance. The armed influenza virus combined the triple advantages of influenza virus, tumor antigen peptide, and immune adjuvant. In this experiment, the effect of the armed influenza virus on tumor microenvironment was investigated.

(1)

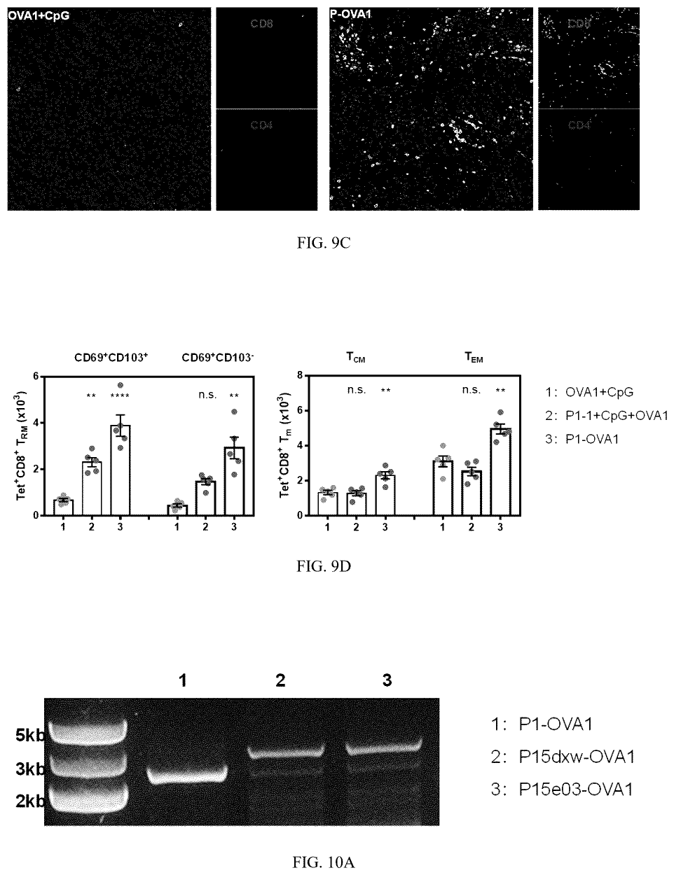

C57BL/6 mice (6 to 8 weeks old, female) were injected with B16-F10-OVA melanoma cells (3×10⁵ cells/200 μl per mouse) via the tail vein. Seven days after the tumor inoculation, the mice were immunized intranasally (10 mice per group) with P1-OVA1 (10⁵ pfu/25 μl per mouse, administered once every 7 days, twice in total) and antigen peptide control (15 μg of OVA1+3 μg of CpG/25 μl per mouse, administered once every 7 days, twice in total). Seven days after the second administration, the lung tumor nodules were harvested, minced, and digested in a buffer containing 2 mg/ml type II and type IV collagenase (GIBCO BRL) and 0.5 mg/ml DNase (Sigma Aldrich) at 37° C. for 13 minutes to form a single cell suspension, then the immune cell subtypes in the tumor microenvironment were determined by flow cytometry, and immunohistochemical analysis of T cell density was performed on the frozen tumor sections at the same time. The administration process was shown in FIG. 9A.

The results were shown in FIGS. 9B to 9D.

The results showed that: on the one hand, P1-OVA1 could significantly increase the contents of IFNγ-positive CD8 T and CD4 T cells and NK cells in tumor tissues, significantly increase the content of OVA-specific CD8 T cells, and at the same time increase the content of DC-level type 1 macrophages (M1); on the other hand, P1-OVA1 could reduce the contents of myeloid-derived suppressor cells (MDSCs), regulatory T cells (Treg) and type 2 macrophages (M2) in the tumor tissues.

The above results showed that the immune-suppressive microenvironment of the tumor was reversed to an immune-active environment after the P1-OVA1 treatment (FIG. 9B). This result was consistent with the immunohistochemical staining, indicating that the P1-OVA1 treatment induced the largest amount of T cell infiltration in tumor tissues as compared with the control group (FIG. 9C).

(2)

Memory CD8 cells play an important role in tumor immunity. In order to study the status of memory CD8 T cells, C57BL/6 mice (6 to 8 weeks old, female) were immunized by intranasal drops (8 mice per group) with P1-OVA1 (10⁵ pfu/25 μl per mouse, administered once every 7 days, twice in total), antigen peptide control (15 μg of OVA1+3 μg of CpG/25 μl per mouse, administered once every 7 days, twice in total) or virus mixed control (10⁵ pfu P1-1+15 μg of OVA1+3 μg of CpG/25 μl per mouse, administered once every 7 days, twice in total). 40 days after the second immunization, the analysis of T cell subsets in lung tissues showed that, as compared with the antigen peptide control group and the virus mixed control group, the number of OVA-specific tissue-resident T cells (T_{RM}) in lung tissue increased significantly (FIG. 9D), and at the same time, the numbers of central memory CD8 T cells (T_{CM}) and effector memory CD8 T cells (T_{EM}) also increased significantly (FIG. 9D), indicating that the armed influenza virus could significantly amplify memory T cells in vivo, especially memory CD8 T cells with antigenic characteristics, which was beneficial to exert anti-tumor immune efficacy.

### Effect Example 5: Experiment of Nanobodies Delivering Anti-Pdl1 and Ctla4

Immune checkpoint inhibitors are currently the most important tumor immunotherapy drugs. As protein drugs, in situ release of immune checkpoint inhibitors at tumor site can avoid drug degradation during circulation in the body and toxicity caused by “on target” and “off tumor” effects. Influenza virus, as an RNA virus, can be used after modification as a delivery carrier for exogenous protein expression. In this experiment, it was investigated whether the armed influenza virus could be used as a delivery carrier for anti-pdl1 or anti-ctla4 nanobody.

A549 cells (ATCC, CRM-CCL-185) were infected with P1-OVA1 or P15dxw-OVA1 or P15e03-OVA1 at MOI=1000. RNA was extracted 8 hours after the infection, and reverse transcription was performed using the 12primer-2 primer in Table 9. The reverse transcription products were subjected to PCR amplification using the PB2-FL-1-F and PB2-FL-1-R primers in Table 9, and the PCR products were identified by agarose gel electrophoresis.

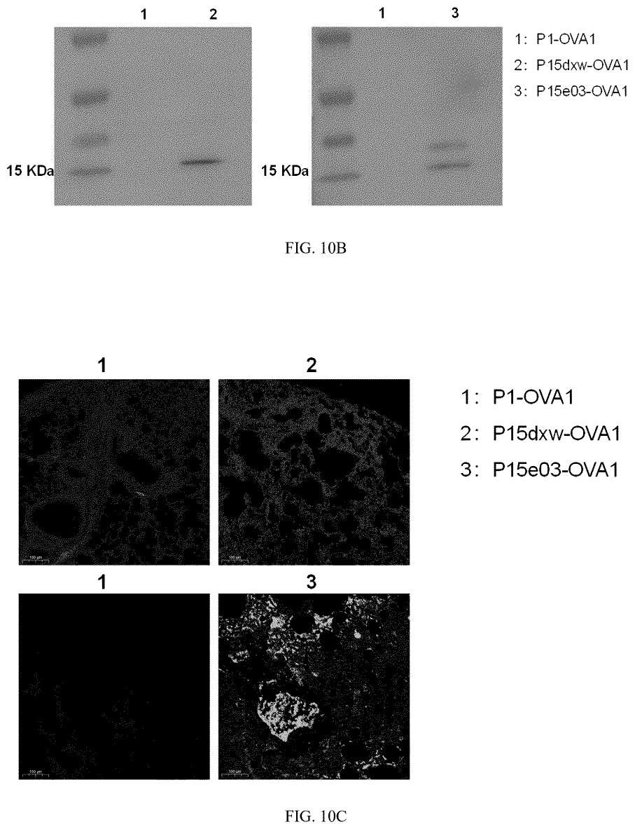

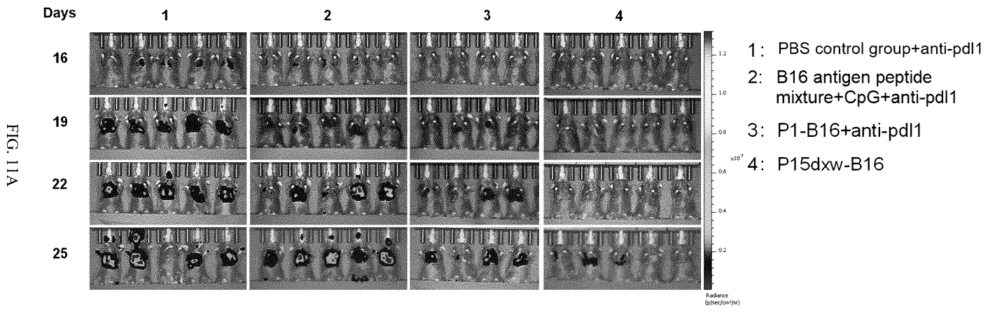

The results were shown in FIG. 10A. The PCR band positions of the P15dxw-OVA1 and P15e03-OVA1 groups were at about 3 kb, which was about 1 kb longer than that of P1-1, which was exactly the length of anti-pdl1 or 5e03 nanobody, proving that the exogenous gene could be packaged into the replication-deficient influenza virus, and that the virus after surface-modification could still infect the cells and deliver the viral RNA.

TABLE 9

| Primer name | Sequence | SEQ ID NO: |
|---|---|---|
| 12primer-2 | AGCGAAAGCAGG | 56 |
| PB2-FL-1-F | AGCGAAAGCAGGTCAATTATATTC | 57 |
| PB2-FL-1-R | GGTCGTTTTTAAACTATTCGACACT | 58 |

In another experiment, A549 cells were infected with P1-OVA1 or P15dxw-OVA1 or P15e03-OVA1 at MOI=1000. After 48 hours of the infection, the cell culture supernatant was collected and detected by western blot. The results were shown in FIG. 10B. The target protein band was detected at about 15 kDa, proving that P15dxw-OVA1 or P15e03-OVA1 could infect cells, express and secret nanobody into the cell culture supernatant.

In another experiment, C57BL/6 mice (6 to 8 weeks old, female) were intranasally administered with P1-OVA1 (10⁵ pfu/25 μl per mouse), P15dxw-OVA1 (10⁵ pfu/25 μl per mouse), and P15e03-OVA1 (10⁵ pfu/25 μl per mouse). After 48 hours, the lung tissues of the mice were taken, the frozen lung tissue sections were subjected to antibody staining for imaging analysis, and the results were shown in FIG. 10C. Compared with the P1-OVA1 group, obvious fluorescent signals could be detected in the P15dxw-OVA1 or P15e03-OVA1 group, proving that P15dxw-OVA1 or P15e03-OVA1 could express and produce antibody in the mice.

### Effect Example 6: Experiment of Combating Pulmonary Metastasis Tumor by Armed Influenza Virus Delivering Anti-Pdl1 Nanobody

The above results showed that P15dxw-OVA1 could produce anti-pdl1 nanobody in mice, especially in the lungs. In this experiment, the effect of combating tumor lung metastasis by this armed influenza virus that could produce nanobody in situ in vivo, especially in lung tumor site, was investigated.

(1) Experiment of Combating Lung Metastasis from Melanoma

In order to make the present invention more widely applicable, a mouse wild-type melanoma B16-F10-luci model was used in this experiment, and a mixed tumor-associated antigen peptide was used to modify the surface of the replication-deficient influenza virus according to Preparation Example 4. The resulting virus was P15dxw-B16.

C57BL/6 mice (6-8 weeks old, female) were injected with B16-F10-luci melanoma cells (ATCC, CRL-6475-LUC2) (3×10⁵ cells/200 μl per mouse) via the tail vein. Seven days after the tumor inoculation, the mice were administered intranasally with PBS control (25 μl per mouse, once every 7 days, twice in total), antigen peptide control (15 μg of B16 antigen peptide mixture+3 μg of CpG/25 μl per mouse, once every 7 days, twice in total), P1-B16 (10⁵ pfu/25 μl per mouse, once every 7 days, twice in total), or P15dxw-B16 (10⁵ pfu/25 μl per mouse, once every 7 days, twice in total). On the 7^{th} and 14^{th} days, the PBS group, antigen peptide control group, and P1-16 group were intraperitoneally injected with anti-pdl1 antibody (Ultra-LEAF™ Purified Anti-mouse CD274 (B7-H1, PD-L1) Antibody, purchased from biolegend, Cat. No. 124338) (100 μg per mouse) (10 mice per group), and the lung tumor growth was recorded by fluorescence imaging every 3 days starting from the 10^{th} day. The mice were killed 28 days after the tumor inoculation, and lung tissues, spleens and peripheral blood were extracted to prepare single cell suspensions. The contents of immune cells in different tissues were then determined by flow cytometry, and immunohistochemical analysis of T cell density was performed on frozen lung tissue sections.

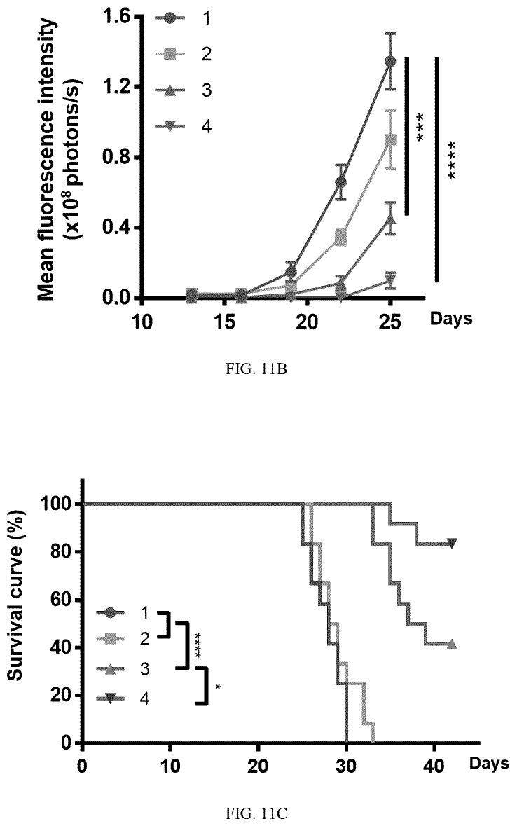

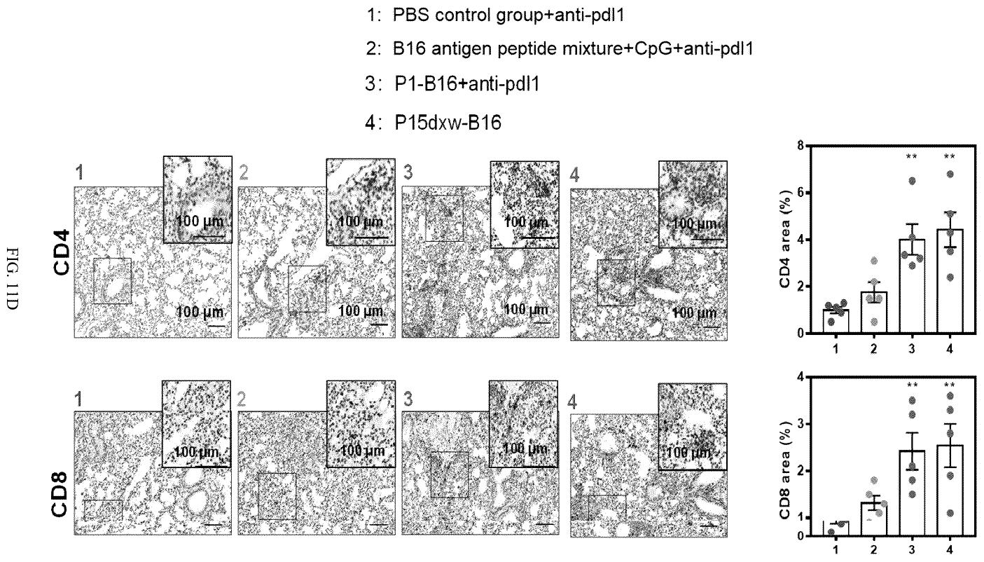

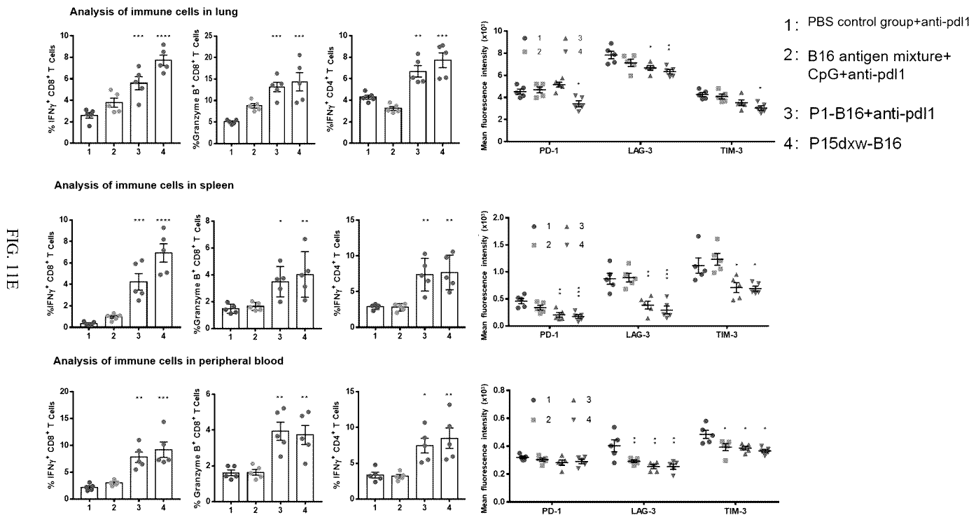

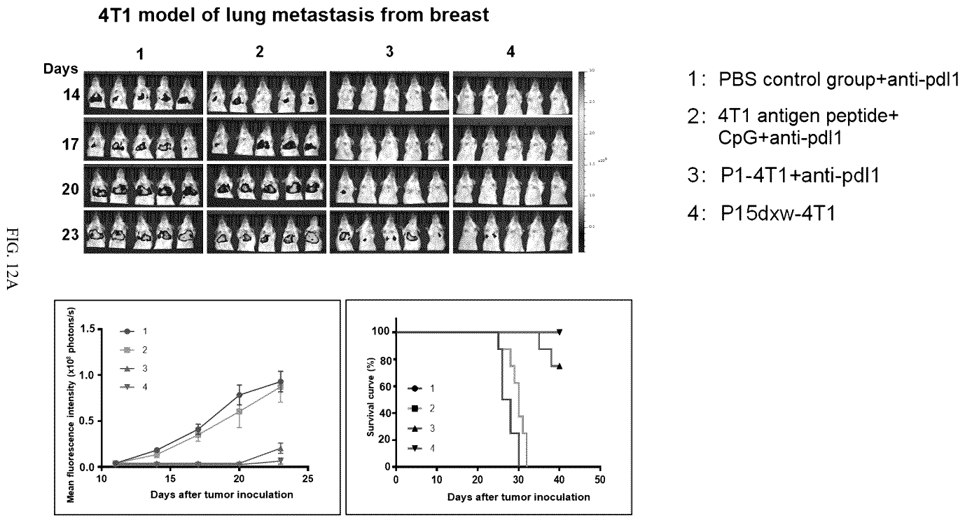

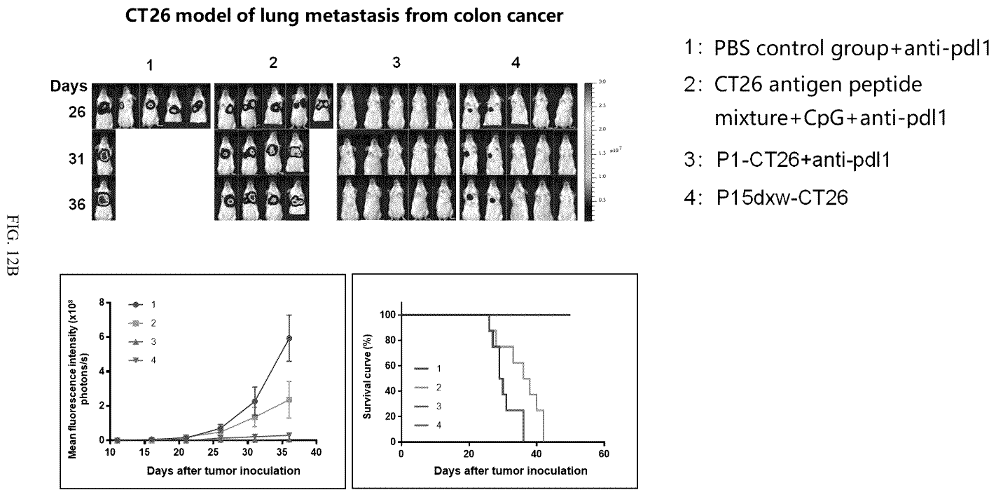

The results were shown in FIGS. 11A to 11E. Compared with the anti-pdl1 alone group or the antigen peptide combined with anti-pdl1 group, the tumors in the P1-16 combined with intraperitoneal injection of anti-pdl1 group were significantly alleviated, and the survival of mice was significantly improved (FIGS. 11A, 11B, and 11C). Compared with the P1-B16 combined with intraperitoneal injection of anti-pdl1 group, the tumors in the P15dxw-B16 treatment group were further inhibited, and the survival of mice was longer, indicating that in situ delivery of anti-pdl1 had a more effective anti-tumor effect than systemic delivery. The results of immunohistochemical staining showed that compared with the anti-pdl1 alone group and the antigen peptide combined with anti-pdl1 group, the infiltration of a large number of T cells was induced in the lung tissues in both the P1-B16 combined with intraperitoneal injection of anti-pdl1 group and the P15dxw-B16 group (FIG. 11D).

Consistent with the results of immunohistochemistry, the results of flow cytometry analysis as shown in FIG. 11E indicated that P15dxw-B16 could significantly increase the contents of IFNγ-positive CD8 and CD4 T cells in the lungs, spleens and peripheral blood of mice, which were consistent with the results of IFNγ-positive CD8 T cells, and P15dxw-B16 also increased the content of granzyme B-positive CD8 T cells. PD-1, LAG-3, and TIM-3 are surface markers of T cell exhaustion. The detection results of CD8 T cells in different tissues showed that P15dxw-B16 could further reduce the expression of PD-1, TIM-3, and LAG-3, and reduce the degree of T cell exhaustion, indicating that in situ release of anti-pdl1 could effectively alleviate the inhibition of tumor cells on T cells, which was more conducive to the anti-tumor effect.

(2) Experiment of Combating Lung Metastasis from Breast Cancer

In order to make the present invention have a wider application, a mouse wild-type breast cancer 4T1-luci model was used in this experiment, and a tumor neoantigen peptide was used to modify the surface of replication-deficient influenza virus according to Preparation Example 4. The resulting virus was P15dxw-4T1.

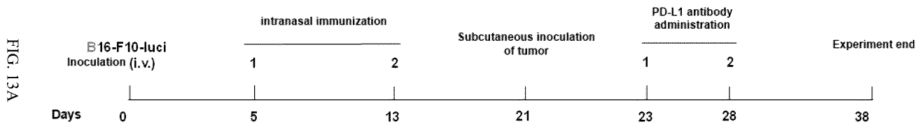

BALB/C mice (6 to 8 weeks old, female) were injected with 4T1-luci breast cancer cells (5×10⁵ cells/200 μl per mouse) via the tail vein. Seven days after the tumor inoculation, the mice were administered intranasally (10 mice per group) with PBS control (25 μl per mouse, once every 7 days, twice in total), antigen peptide control (15 μg of 4T1 neoantigen peptide+3 μg of CpG/25 μl per mouse, once every 7 days, twice in total), P1-4T1 (10⁵ pfu/25 μl per mouse, once every 7 days, twice in total), or P15dxw-4T1 (10⁵ pfu/25 μl per mouse, once every 7 days, twice in total). On the 7^{th} and 14^{th} days, the PBS group, the antigen peptide control group, and the P1-4T1 group were intraperitoneally injected with anti-pdl1 antibody (Ultra-LEAF™ Purified anti-mouse CD274 (B7-H1, PD-L1) Antibody, purchased from biolegend, Cat. No. 124338) (100 g per mouse), and the lung tumor growth was recorded by fluorescence imaging every 3 days starting from the 11^{th} day. The results were shown in FIG. 12A. Compared with the anti-pdl1 alone group and the antigen peptide combined with anti-pdl1 group, the P1-4T1 combined with anti-pdl1 group and the P15dxw-4T1 group both showed significant anti-tumor effect, and the mice in the P15dxw-4T1 group had a longer survival period than those in the P1-4T1 combined with anti-pdl1 group.

(3) Experiment of Combating Lung Metastasis from Colon Cancer

In order to make the present invention have a wider application, a mouse wild-type colon cancer CT26-luci model was used in this experiment, and a tumor neoantigen peptide mixture was used to modify the surface of the replication-deficient influenza virus according to Preparation Example 4, and the resulting virus was P15dxw-CT26.

BALB/C mice (6 to 8 weeks old, female) were injected with CT26-luci breast cancer cells (1×10⁶ cells/200 μl per mouse) via the tail vein. Seven days after the tumor inoculation, the mice were administered intranasally (10 mice per group) with PBS control (25 μl per mouse, once every 7 days, twice in total), antigen peptide control (15 μg of CT26 neoantigen peptide mixture+3 μg of CpG/25 μl per mouse, once every 7 days, twice in total), P1-CT26 (10⁵ pfu/25 μl per mouse, once every 7 days, twice in total), or P15dxw-CT26 (10⁵ pfu/25 μl per mouse, once every 7 days, twice in total). On the 7^{th} and 14^{th} days, the PBS group, the antigen peptide control group, and the P1-CT26 group were intraperitoneally injected with anti-pdl1 antibody (Ultra-LEAF™ Purified anti-mouse CD274 (B7-H1, PD-L1) Antibody, purchased from biolegend, Cat. No. 124338) (100 μg per mouse), and the lung tumor growth was recorded by fluorescence imaging every 5 days starting from the 11^{th} day. The results were shown in FIG. 12B. Compared with the anti-pdl1 alone group and the antigen peptide combined with anti-pdl1 group, the P1-4T1 combined with anti-pdl1 group and the P15dxw-4T1 group both showed significant anti-tumor effect, and by the end of the experiment, no mouse deaths were found in the P1-4T1 combined with anti-pdl1 group and the P15dxw-4T1 group.

The above results showed that the armed influenza virus could produce a synergistic anti-tumor effect with the anti-pdl1 antibody. In addition, the delivery of anti-pdl1 nanobody by the armed influenza virus could effectively treat various types of lung metastatic tumors, and the in situ delivery of immune checkpoint inhibitor was superior to the systemic delivery of combination therapy.

### Effect Example 7: Experiment of Combating Pulmonary Metastasis Tumor and Distal Tumor By Armed Influenza Virus Delivering Anti-Pdl1 Nanobody

According to the results of Effect Example 6, the mice treated with P15dxw-B16 had significant increases in anti-tumor immune cells detected in the spleen and peripheral blood, and it could be seen that the intranasal immunization with P15dxw-B16 or P1-B16 could produce a systemic immune enhancement effect. Therefore, in this experiment, it was investigated whether this systemic immune enhancement effect would bring about a therapeutic effect on distal tumors other than lung tumors.

C57BL/6 mice (6 to 8 weeks old, female) were injected with B16-F10-luci melanoma cells (3×10⁵ cells/200 μl per mouse) via the tail vein. Five days after the tumor inoculation, the mice were administered intranasally (10 mice per group) with PBS control (25 μl per mouse, once every 7 days, twice in total), antigen peptide control (15 μg of B16 antigen peptide mixture+3 μg of CpG/25 μl per mouse, once every 7 days, twice in total) or P15dxw-B16 (10⁵ pfu/25 μl per mouse, once every 7 days, twice in total). The mice were inoculated subcutaneously with B16-F10-luci melanoma cells (5×10⁵ cells per mouse) on the 21^{st} day, and intraperitoneally injected with anti-pdl1 antibody (Ultra-LEAF™ Purified anti-mouse CD274 (B7-H1, PD-L1) Antibody, purchased from biolegend, Cat. No. 124338) (100 μg per mouse) on the 23^{rd} and 28^{th} days. Tumor volume was measured every day after the subcutaneous inoculation. The mice were killed on the 38^{th} day, mouse tumor tissues were extracted, the tumors were minced and digested in a buffer containing 2 mg/ml type II and IV collagenase (GIBCO BRL) and 0.5 mg/ml DNase (Sigma Aldrich) at 37° C. for 13 minutes to form a single cell suspension, and then the immune cell subtypes in the tumor microenvironment were determined by flow cytometry.

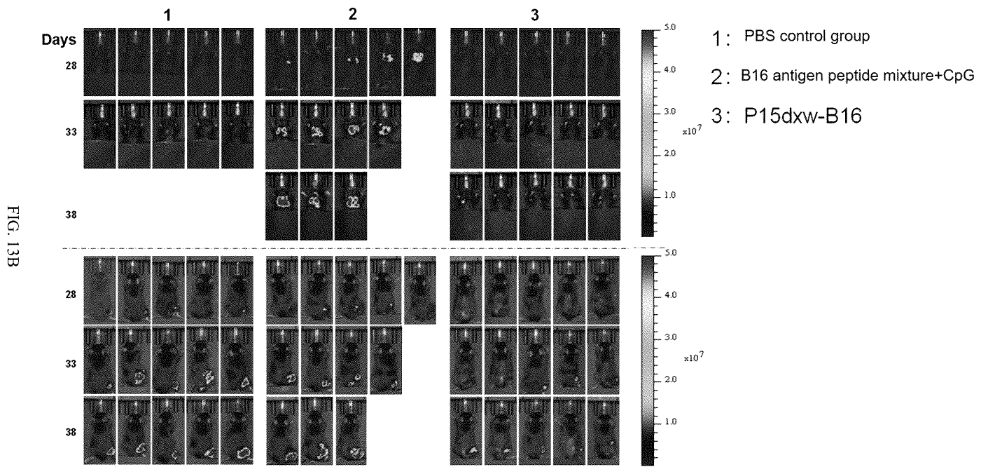

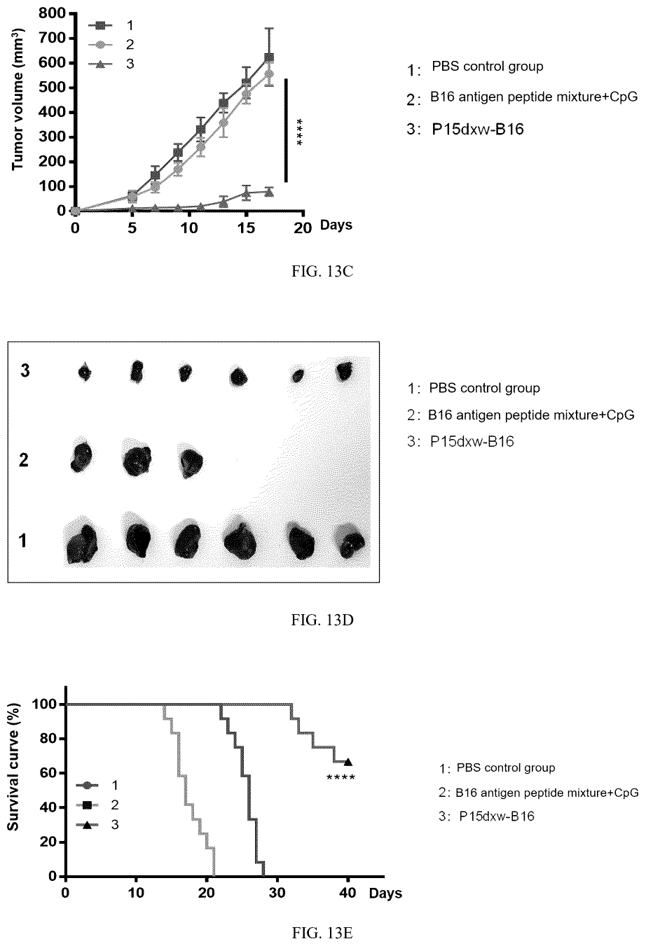

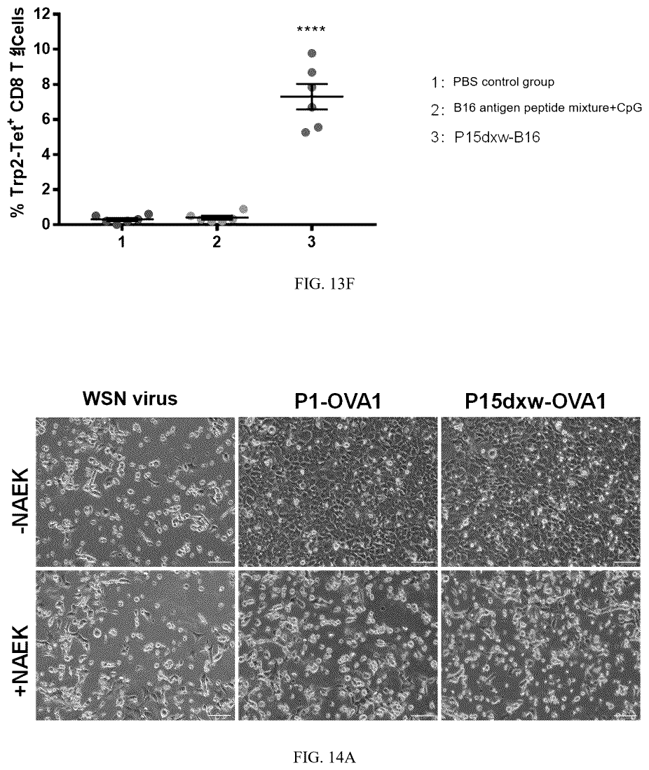

The results were shown in FIGS. 13A to 13F. The P15dxw-B16 combined with anti-pdl1 antibody treatment group could significantly inhibit the growth of lung and distal tumors and prolong the survival period of mice (FIGS. 13B to 13E). The results of flow cytometry analysis of T cells in tumor tissues were shown in FIG. 13F. A large number of Trp2 antigen-positive CD8 T cells could be detected in the tumors of the P15dxw-B16 combined with anti-pdl1 antibody treatment group, indicating that the P15dxw-B16 combined with anti-pdl1 antibody treatment could generate systemic CD8 T cells against exogenous antigen peptide in mice, thereby facilitating the treatment of systemic spread of tumors.

### Effect Example 8: Experiment to Prove Safety of Armed Influenza Virus

HEK293-PYL cells were infected with wild-type WSN virus, P1-OVA1 or P15dxw-OVA1 at MOI=100, and experimental groups with or without NAEK in the culture medium were set up.

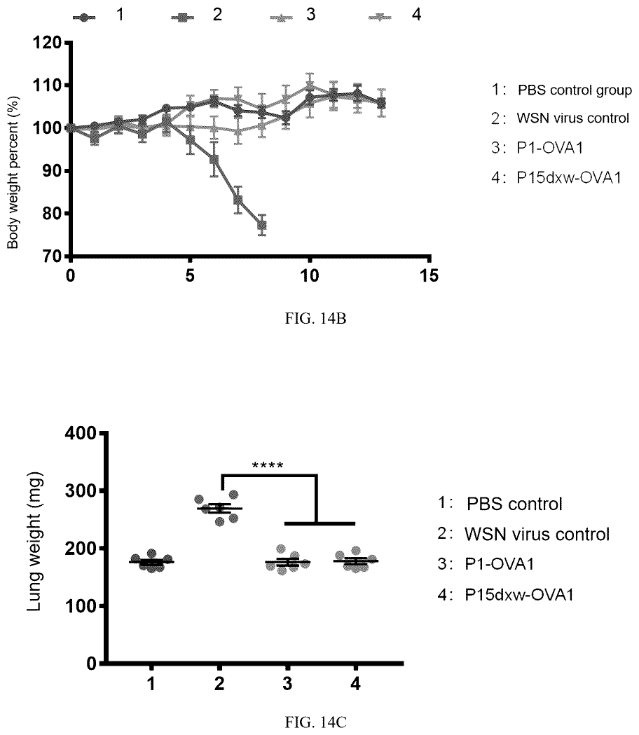

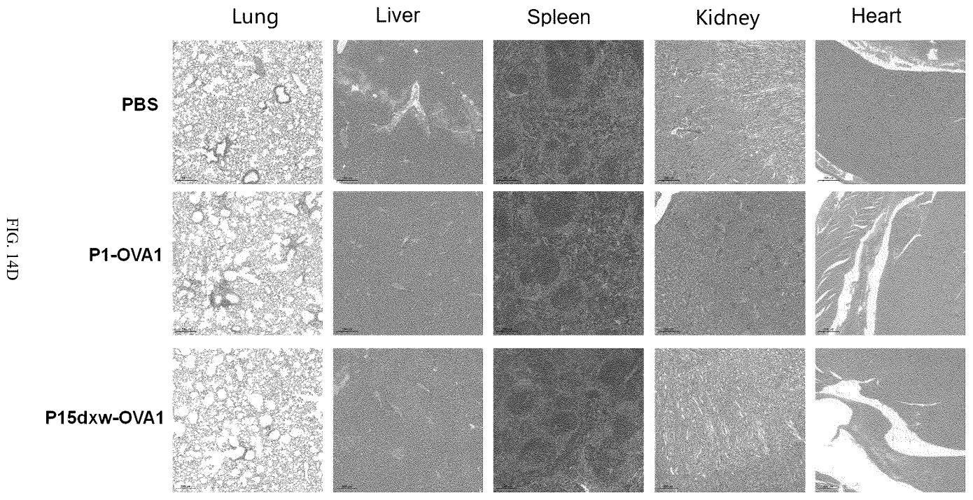

The experimental results were shown in FIG. 14A. Regardless of whether the culture medium contained NAEK, the wild-type WSN virus would cause cell death, while in the P1-OVA1 group and the P15dxw-OVA1 group, cell death would occur only when NAEK was present, and replication would not occur in the absence of NAEK, indicating that the armed influenza virus was absolutely safe at the cellular level.

The mouse median lethal dose LD50 of influenza virus WSN was 10⁴ virus particles/25 μl. BALB/C mice (6 to 8 weeks old, female) were inoculated with PBS (25 μl per mouse), WSN (2×10⁴/25 μl per mouse), P1-OVA1 (10⁶/25 μl per mouse) or P15dxw-OVA1 (10⁶/25 μl per mouse) (8 mice per group) by intranasal inoculation. The body weight was measured every day after the inoculation. The mice were killed 14 days later, and the lung tissues of the mice were weighed. At the same time, the frozen lung, liver, spleen, kidney and heart sections were subjected to H&E staining analysis.

As shown in FIGS. 14B to 14D, the weight of mice in the WSN virus inoculation group decreased significantly, and mice began to die 8 days after the virus inoculation, while the weight of mice in the P1-OVA1 and P15dxw-OVA1 inoculation groups did not change significantly (FIG. 14B). Consistent with the weight results, the lungs of mice inoculated with WSN virus showed obvious edema and increased weight due to the WSN virus infection, while the lungs of mice in the P1-OVA1 and P15dxw-OVA1 inoculation groups did not have edema (FIG. 14C). The results of H&E staining of tissue sections showed that no lesions appeared in the organs of mice in the P1-OVA1 and P15dxw-OVA1 inoculation groups, proving the safety of the armed influenza virus (FIG. 14D).

Although the specific embodiments of the present invention have been described in detail, those skilled in the art will understand that various modifications and substitutions can be made to those details based on all the teachings that have been disclosed, and these changes are within the scope of protection of the present invention. The full scope of the present invention is given by the appended claims and any equivalents thereof.

1. A mutated influenza virus, which comprises a nucleic acid encoding HA protein and/or a nucleic acid encoding NA protein, wherein the nucleic acid encoding HA protein and/or the nucleic acid encoding NA protein contains one or more UAG codons.

2. The mutated influenza virus according to claim 1, characterized in one or more of the following:
(1) wherein the nucleic acid comprises a codon mutated to UAG, wherein the codon encoding a site of HA protein selected from one or more of the following:
C84, S86, S92, S126, E132, P135, G147, K170, K176, N179, S201, 1256, S53, K57, K62, I64, A65, L67, K71 or P82;
(2) the mutated influenza virus which further comprises a nucleic acid encoding PB1 protein, PA protein and/or NP protein, wherein the nucleic acid encoding PB1 protein, PA protein and/or NP protein contains one or more UAG codons;
(3) the nucleic acid codon encoding the following site is mutated to UAG codon:
R52 site of PB1 protein,
R266 site of PA protein,
and/or
D101 site of NP protein;
(4) the nucleic acid codons encoding the following sites are mutated to UAG codons:
R52 site of PB1 protein, R266 site of PA protein, S53 site of HA protein and D101 site of NP protein;
K33 site of PB2 protein, R266 site of PA protein, S53 site of HA protein and D101 site of NP protein;
or
K33 site of PB2 protein, R52 site of PB1 protein, S53 site of HA protein and D101 site of NP protein;
(5) before mutation the influenza virus is a wild-type influenza virus: preferably, a wild-type influenza virus A/WSN/1933;
(6) the amino acid sequence of the PB2 protein unmutated is the same as the amino acid sequence encoded by SEQ ID NO: 1;
the amino acid sequence of the PB1 protein unmutated is the same as the amino acid sequence encoded by SEQ ID NO: 2;
the amino acid sequence of the PA protein unmutated is the same as the amino acid sequence encoded by SEQ ID NO: 3;
the amino acid sequence of the HA protein unmutated is the same as the amino acid sequence encoded by SEQ ID NO: 4;
the amino acid sequence of the NP protein unmutated is the same as the amino acid sequence encoded by SEQ ID NO: 5;
the amino acid sequence of the NA protein unmutated is the same as the amino acid sequence encoded by SEQ ID NO: 6;
the amino acid sequence of the M protein unmutated is the same as the amino acid sequence encoded by SEQ ID NO: 7;
and/or
the amino acid sequence of the NS protein unmutated is the same as the amino acid sequence encoded by SEQ ID NO: 8;
(7) one or more of the UAG codons are located upstream of stop codon;
(8) the amino acids at the positions encoded by one or more of the UAG codons are identical or different non-natural amino acids, such as NAEK.

3-9. (canceled)

10. A mutated influenza virus, wherein the following sites are mutated to non-natural amino acids, such as NAEK:
R52 site of PB1 protein, R266 site of PA protein, S53 site of HA protein and D101 site of NP protein;
K33 site of PB2 protein, R266 site of PA protein, S53 site of HA protein and D101 site of NP protein;
or
K33 site of PB2 protein, R52 site of PB1 protein, S53 site of HA protein and D101 site of NP protein.

11. A recombinant influenza virus, which is obtainable by recombining the mutated influenza virus according to claim 1 with an exogenous nucleic acid;
preferably, the exogenous nucleic acid is inserted into the nucleic acid encoding PB2, PB1 or PA;
preferably, the exogenous nucleic acid is a nucleic acid encoding an anti-tumor antibody;
preferably, the anti-tumor antibody is an anti-PD-1 antibody, an anti-PD-L1 antibody and/or an anti-CTLA-4 antibody;
preferably, the anti-tumor antibody is a nanobody, a single-chain antibody, a monoclonal antibody or a bispecific antibody;
preferably, the anti-tumor antibody is an anti-PD-1 nanobody or an anti-CTLA-4 nanobody;
preferably, the amino acid sequence of the anti-PD-1 nanobody is shown in SEQ ID NO: 9;
preferably, the amino acid sequence of the anti-CTLA-4 nanobody is shown in SEQ ID NO: 10.

12. A modified influenza virus, wherein one or more antigen peptides for anti-tumor are attached to the surface of the mutated influenza virus according to claim 1.

13. The modified influenza virus according to claim 12 characterized in one or more of the following:
(1) the antigen peptide is linked to HA protein or NA protein on the surface of the virus; preferably, the antigen peptide is linked to a non-natural amino acid such as NAEK of HA protein or NA protein;
(2) the antigen peptide is linked to a non-natural amino acid of HA protein in the form of any compound selected from the compounds as shown in Formula III-1 to Formula III-6: 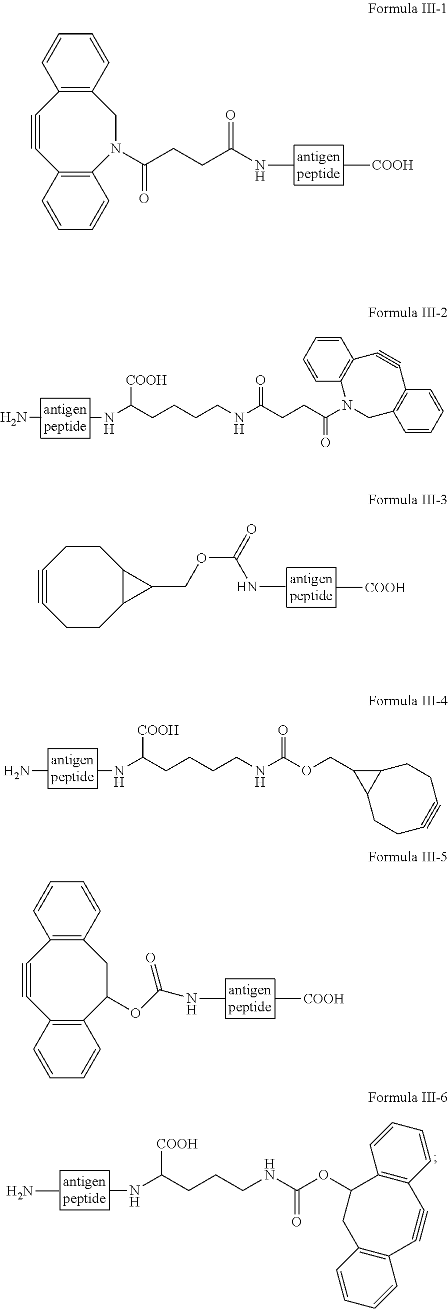
(3) the amino acid sequence of the antigen peptide is independently selected from any one of sequences as shown in SEQ ID NOs: 49-55;
(4) a CpG adjuvant is further attached to the surface of the influenza virus;
preferably, the sequence of the CpG adjuvant is shown in SEQ ID NO: 20;
preferably, the CpG adjuvant is attached to the virus surface in the form of a compound as shown in Formula IV,

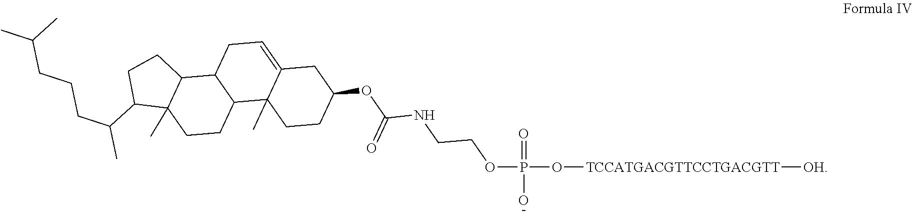

14.-16. (canceled)

17. A pharmaceutical composition, which comprises the modified influenza virus according to claim 12;
optionally, the pharmaceutical composition further comprises one or more pharmaceutically acceptable excipients;
preferably, the pharmaceutical composition is a vaccine composition;
preferably, the vaccine composition is an anti-tumor vaccine composition;
preferably, the tumor is selected from the group consisting of primary lung tumor and lung metastatic tumor;
preferably, the tumor is one or more selected from the group consisting of lung cancer (small cell lung cancer or non-small cell lung cancer), melanoma lung metastatic tumor, breast cancer lung metastatic tumor, and colon cancer lung metastatic tumor.

18. (canceled)

19. A method for treating or preventing a tumor, comprising a step of administering to a subject in need thereof an effective amount of the modified influenza virus according tom claim 12;
preferably, the tumor is selected from the group consisting of primary lung tumor and lung metastasis tumor;
preferably, the tumor is one or more selected from the group consisting of lung cancer (small cell lung cancer or non-small cell lung cancer), lung metastasis from melanoma, lung metastasis from breast cancer, and lung metastasis from colon cancer.

20. (canceled)

21. A plasmid-based influenza virus reverse genetics system, comprising:
(1) 8 recombinant plasmids containing PB2 gene, PB1 gene, PA gene, HA gene, NP gene, NA gene, M gene and NS gene, respectively, and a vector, and
(2) 4 additional recombinant plasmids containing PB2 gene, PB1 gene, PA gene and NP gene, respectively, and a vector;
wherein the vector used for the recombinant plasmid in (2) is different from the vector used for the recombinant plasmid in (1); and the PB2 gene, PB1 gene, PA gene and NP gene in (2) are wild type;
wherein, the HA gene and/or NA gene contain one or more TAG codons.

22. The plasmid-based influenza virus reverse genetics system according to claim 21, characterized in one or more of the following:
(1) the nucleic acid comprises a codon mutated to TAG, wherein the codon encoding a site of HA protein selected from one or more of the following:
C84, S86, S92, S126, E132, P135, G147, K170, K176, N179, S201, 1256, S53, K57, K62, 164, A65, L67, K71 or P82
(2) the PB1 gene, PA gene and/or NP gene in (1) contain one or more TAG codons;
(3) the nucleic acid codon encoding the following site is mutated into TAG codon:
R52 site of PB1 protein,
R266 site of PA protein,
and/or
D101 site of NP protein;
(4) the nucleic acid codons encoding the following sites are mutated into TAG codons:
R52 site of PB1 protein, R266 site of PA protein, S53 site of HA protein and D101 site of NP protein;
K33 site of PB2 protein, R266 site of PA protein, S53 site of HA protein and D101 site of NP protein;
or
K33 site of PB2 protein, R52 site of PB1 protein, S53 site of HA protein and D101 site of NP protein;
(5) before mutation the influenza virus is a wild-type influenza virus: preferably, a wild-type influenza A/WSN/1933;
(6) the nucleic acid sequence of the PB2 gene unmutated is shown in SEQ ID NO: 1;
the nucleic acid sequence of the PB1 gene unmutated is shown in SEQ ID NO: 2;
the nucleic acid sequence of the PA gene unmutated is shown in SEQ ID NO: 3;
the nucleic acid sequence of the HA gene unmutated is shown in SEQ ID NO: 4;
the nucleic acid sequence of the NP gene unmutated is shown in SEQ ID NO: 5;
the nucleic acid sequence of the NA gene unmutated is shown in SEQ ID NO: 6;
the nucleic acid sequence of the M gene unmutated is shown in SEQ ID NO: 7;
and/or
the nucleic acid sequence of the NS gene unmutated is shown in SEQ ID NO: 8;
(7) one or more of the TAG codons are located upstream of stop codon;
(8) the amino acids at the positions encoded by one or more of the TAG codons are identical or different non-natural amino acids, such as NAEK.

23.-29. (canceled)

30. An influenza virus, which is rescued by the plasmid-based influenza virus reverse genetics system according to claim 21.

31. A recombinant influenza virus, which is obtainable by recombining the mutated influenza virus according to claim 10 with an exogenous nucleic acid;
preferably, the exogenous nucleic acid is inserted into the nucleic acid encoding PB2, PB1 or PA;
preferably, the exogenous nucleic acid is a nucleic acid encoding an anti-tumor antibody;
preferably, the anti-tumor antibody is an anti-PD-1 antibody, an anti-PD-L1 antibody and/or an anti-CTLA-4 antibody;
preferably, the anti-tumor antibody is a nanobody, a single-chain antibody, a monoclonal antibody or a bispecific antibody;
preferably, the anti-tumor antibody is an anti-PD-1 nanobody or an anti-CTLA-4 nanobody;
preferably, the amino acid sequence of the anti-PD-1 nanobody is shown in SEQ ID NO: 9;
preferably, the amino acid sequence of the anti-CTLA-4 nanobody is shown in SEQ ID NO: 10.

32. A modified influenza virus, wherein one or more antigen peptides for anti-tumor are attached to the surface of the mutated influenza virus according to claim 10.

33. The modified influenza virus according to claim 32, characterized in one or more of the following:
(1) the antigen peptide is linked to HA protein or NA protein on the surface of the virus; preferably, the antigen peptide is linked to a non-natural amino acid such as NAEK of HA protein or NA protein;
(2) the antigen peptide is linked to a non-natural amino acid of HA protein in the form of any compound selected from the compounds as shown in Formula III-1 to Formula III-6: 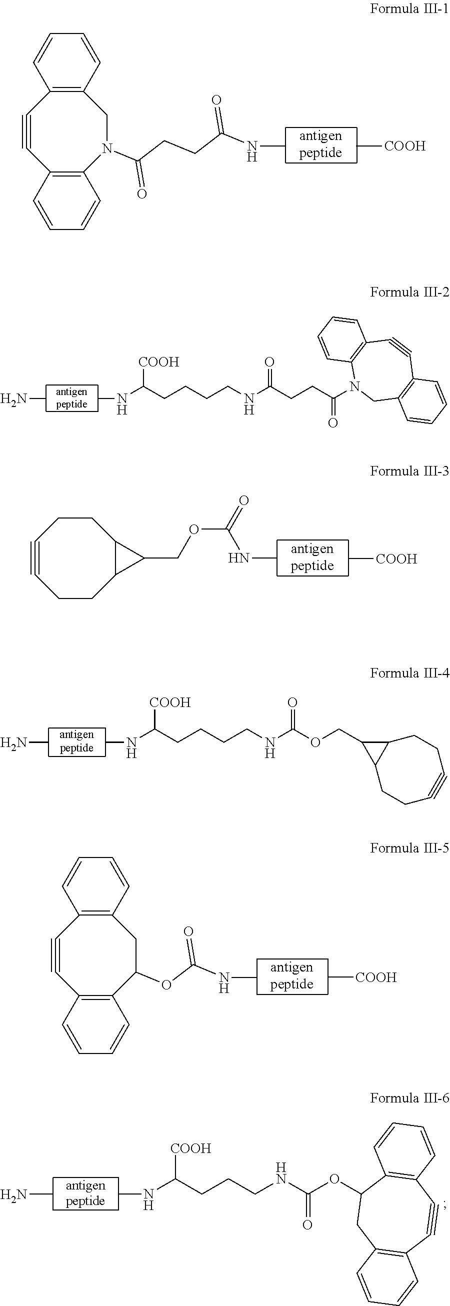
(3) the amino acid sequence of the antigen peptide is independently selected from any one of sequences as shown in SEQ ID NOs: 49-55;
(4) a CpG adjuvant is further attached to the surface of the influenza virus;
preferably, the sequence of the CpG adjuvant is shown in SEQ ID NO: 20;
preferably, the CpG adjuvant is attached to the virus surface in the form of a compound as shown in Formula IV,

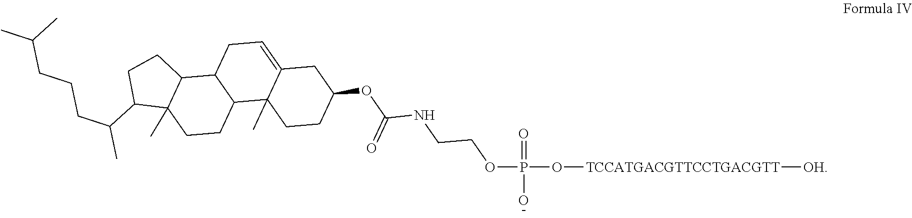

34. A modified influenza virus, wherein one or more antigen peptides for anti-tumor are attached to the surface of the recombinant influenza virus according to claim 11.

35. A modified influenza virus, wherein one or more antigen peptides for anti-tumor are attached to the surface of the recombinant influenza virus according to claim 31.

36. A pharmaceutical composition, which comprises the modified influenza virus according to claim 32;
optionally, the pharmaceutical composition further comprises one or more pharmaceutically acceptable excipients;
preferably, the pharmaceutical composition is a vaccine composition;
preferably, the vaccine composition is an anti-tumor vaccine composition;
preferably, the tumor is selected from the group consisting of primary lung tumor and lung metastatic tumor;
preferably, the tumor is one or more selected from the group consisting of lung cancer (small cell lung cancer or non-small cell lung cancer), melanoma lung metastatic tumor, breast cancer lung metastatic tumor, and colon cancer lung metastatic tumor.

37. A pharmaceutical composition, which comprises the modified influenza virus according to claim 35;
optionally, the pharmaceutical composition further comprises one or more pharmaceutically acceptable excipients;
preferably, the pharmaceutical composition is a vaccine composition;
preferably, the vaccine composition is an anti-tumor vaccine composition;
preferably, the tumor is selected from the group consisting of primary lung tumor and lung metastatic tumor;
preferably, the tumor is one or more selected from the group consisting of lung cancer (small cell lung cancer or non-small cell lung cancer), melanoma lung metastatic tumor, breast cancer lung metastatic tumor, and colon cancer lung metastatic tumor.

38. A method for treating or preventing a tumor, comprising a step of administering to a subject in need thereof an effective amount of the modified influenza virus according to claim 32;
preferably, the tumor is selected from the group consisting of primary lung tumor and lung metastasis tumor;
preferably, the tumor is one or more selected from the group consisting of lung cancer (small cell lung cancer or non-small cell lung cancer), lung metastasis from melanoma, lung metastasis from breast cancer, and lung metastasis from colon cancer.

39. A method for treating or preventing a tumor, comprising a step of administering to a subject in need thereof an effective amount of the modified influenza virus according to claim 35;
preferably, the tumor is selected from the group consisting of primary lung tumor and lung metastasis tumor;
preferably, the tumor is one or more selected from the group consisting of lung cancer (small cell lung cancer or non-small cell lung cancer), lung metastasis from melanoma, lung metastasis from breast cancer, and lung metastasis from colon cancer.
